(19) United States
(12) Patent Application Publication
(10) Pub. No.: US 2025/0283169 A1
(43) Pub. Date: Sep. 11, 2025

(54) ENHANCED RESOLUTION IMAGING

(71) Applicant: ULTIMA GENOMICS, INC., Fremont, CA (US)

(72) Inventors: Osip Schwartz, Lafayette, CA (US); Gilad Almogy, Palo Alto, CA (US); Rongwen Lu, Fremont, CA (US); Gene Polovy, Redwood City, CA (US); Amy Elizabeth Frantz, Cherry Hill, NJ (US); Michael Friedmann, Fremont, CA (US)

(73) Assignee: ULTIMA GENOMICS, INC., Fremont, CA (US)

(21) Appl. No.: 19/090,897

(22) Filed: Mar. 26, 2025

Related U.S. Application Data
(63) Continuation of application No. PCT/US2023/034377, filed on Oct. 3, 2023.
(60) Provisional application No. 63/413,224, filed on Oct. 4, 2022.

(51) Int. Cl. C12Q 1/6874; G01N 21/64

(52) U.S. Cl. CPC C12Q 1/6874; G01N 21/6428; G01N 21/6458

(57) ABSTRACT

Provided herein are systems and methods that combine the use of first and second optical transformations (e.g., implemented using optical photon reassignment (OPRA)) with time delay and integration (TDI) imaging and external illumination to provide high throughput imaging while maintaining a high signal to noise ratio and providing enhanced image resolution.

### CROSS-REFERENCE TO RELATED APPLICATIONS

This application is a continuation of International Patent Application No. PCT/US2023/034377 filed Oct. 3, 2023, which claims the priority benefit of U.S. Provisional Patent Application Ser. No. 63/413,224, filed on Oct. 4, 2022. The contents of each of the foregoing applications are incorporated by reference herein in their entirety.

### FIELD OF THE DISCLOSURE

The present disclosure relates generally to methods and systems for enhanced resolution imaging, and more specifically to methods and systems for performing enhanced resolution imaging for bioassay applications, e.g., nucleic acid detection and sequencing applications.

### BACKGROUND OF THE DISCLOSURE

High performance imaging systems used for optical inspection are designed to maximize imaging throughput, signal-to-noise ratio (SNR), image resolution, and image contrast, key figures of merit for many imaging applications. In genome sequencing, for example, high resolution imaging enables the use of higher packing densities of nucleic acids (e.g., clonally amplified nucleic acid molecules) on a surface, which in turn may enable higher throughput sequencing in terms of the number of bases called per sequencing reaction cycle.

However, attempting to increase imaging throughput while simultaneously trying to improve the ability to resolve small image features at higher magnification may result in a reduced number of photons available for imaging. In fluorescence imaging-based sequencing, for example, where fluorophores are used to label nucleic acid molecules tethered to a surface, high resolution imaging may in effect reduce the total number of fluorophores present in the region of the surface being imaged, and thus result in the generation of fewer photons. Although this problem may be addressed, for example, by integrating over longer periods of time to acquire an acceptable image (e.g., an image that has a sufficient signal-to-noise ratio to resolve the features of interest), this approach may have an adverse effect on image data acquisition rates, imaging throughput, and overall sequencing reaction cycle times.

There is thus a need for imaging methods that can combine high throughput processing (e.g., high density arrays of samples, high-speed imaging, etc.), high resolution, and high resolution, especially for use in sequencing methods.

### SUMMARY

The image resolution of conventional imaging systems is limited by diffraction to a value determined by the effective numerical aperture (NA) of the object-facing imaging optics and the wavelength of light being imaged. In recent years, many imaging techniques (e.g., stimulated emission depletion microscopy (STED), photo-activated localization microscopy (PALM), stochastic optical reconstruction microscopy (STORM), reversible saturable optical fluorescence transitions microscopy (RESOLFT), etc.) have been developed that may be used to acquire images that exceed diffraction-limited image resolution. However, these approaches generally have low imaging throughput (and in many cases require specialized fluorophores), thus precluding their use for high-speed imaging applications.

Other imaging techniques (e.g., confocal microscopy, structured illumination microscopy (SIM), and image scanning microscopy (ISM)) that may be used to acquire images of more modest but still significant increases in image resolution utilize patterned illumination. However, these techniques either suffer from a significant loss of signal in view of the modest increase in resolution obtained (e.g., due to use of pinhole apertures as spatial filters in the case of confocal microscopy) or require the acquisition of multiple images and a subsequent computational reconstruction of a resolution-enhanced image (thereby significantly increasing image acquisition times, imaging system complexity, and computational overhead for structured illumination microscopy (SIM) and image scanning microscopy (ISM)). Having to acquire multiple images also generally has the undesirable effect that read noise (or digitization noise) is accumulated with every image acquisition.

Time delay and integration (TDI) imaging enables a combination of high throughput imaging with high SNR by accumulating the image-forming signal onto a two-dimensional stationary sensor pixel array that shifts the acquired image signal from one row of pixels in the pixel array to the next synchronously with the motion of an object being imaged as it is moved relative to the imaging system, or vice versa. As is the case with conventional imaging systems, the image resolution for conventional TDI imaging systems is diffraction limited. Thus, there remains an unmet need for an imaging system capable of high-throughput imaging while simultaneously maintaining high image resolution, high SNR, and high image contrast.

Disclosed herein are systems and methods that combine: (i) the use of a first optical transformation to create patterned illumination that is directed to an imaged object such that light reflected, transmitted, scattered, or emitted by the object comprises high-resolution spatial information about the object that would not otherwise be obtained, (ii) the use of a second optical transformation that generates an enhanced resolution optical image at a time delay and integration (TDI) image sensor that comprises all or a portion of the high-resolution information contained in said light due to the patterned illumination, and (iii) the use of illumination light at an NA greater than that permitted by an objective of the system to achieve increased resolution. The resulting enhanced-resolution images can be acquired without requiring a change in the configuration, position, or orientation of the optical transformation devices used to generate the first and second optical transformations, with no additional digital processing required, or, in some instances, using digital processing of substantially reduced computational complexity in comparison with conventional enhanced resolution imaging methods. In addition, with an increase in resolution, it is possible to image a denser array of analytes than is possible with conventional imaging systems and methods. All these factors contribute to the simplicity and high throughput of the imaging system.

In one exemplary implementation, the disclosed systems and methods utilize a novel combination of optical photon reassignment (OPRA) with time delay and integration (TDI) imaging and external illumination to provide high-throughput and high signal-to-noise ratio (SNR) images of an object while also providing enhanced image resolution. The disclosed systems and methods provide enhanced image resolution without compromising the imaging throughput and high SNR achieved using TDI imaging by incorporating passive optical transformation device(s) into both the illumination and detection optical paths of the imaging system and utilizing illumination light with a high NA to increase the density of illumination light intensity maxima. In some embodiments, the systems and methods described herein provide enhanced image resolution (e.g., enhanced raw image resolution) as compared to that for images acquired using an otherwise identical imaging system that lacks one or more of the passive optical transformation devices and/or the external illumination. In some embodiments, the enhanced-resolution image is obtained in a single scan, without the need to acquire or recombine multiple images. In some embodiments, the enhanced-resolution images are produced with little or no digital processing required.

Provided herein are systems and methods that address at least the abovementioned concerns. Disclosed herein are systems and methods for increasing the density of illumination patterns (e.g., light intensity maxima) on substrates and achieving corresponding increases in detection resolution. Beneficially, disclosed systems and methods result in improved resolution over conventional methods. The systems and methods provided herein, in some embodiments, may be standalone systems or may be incorporated into pre-existing imaging systems. In some embodiments, the imaging systems may be useful for imaging, for example, biological analytes, non-biological analytes, synthetic analytes, cells, tissue samples, or any combination thereof.

In an aspect, provided is an imaging method, comprising: a) providing a substrate, wherein the substrate is substantially planar; b) illuminating a region of the substrate with one or more illumination beams, wherein the one or more illumination beams are not directed through an objective lens; and c) directing emission light from the region of the substrate to a detector through the objective lens, thereby generating a scanned image of the region of the substrate, wherein the emission light is directed through an optical transformation device prior to being received by the detector.

In some embodiments, the substrate comprises a first and second surface, wherein the first surface is closer to the objective lens than the second surface. In some embodiments, the first surface and the second surface are parallel to each other, and the substrate is positioned normal to the objective lens.

In some embodiments, the one or more illumination beams are incident on the first surface of the substrate. In some embodiments, the one or more illumination beams are incident on the second surface of the substrate. In some embodiments, each of the one or more illumination beams are transmitted through a liquid immersion coupler prior to illuminating the substrate. In some embodiments, the liquid immersion couplers comprise prism couplers. In some embodiments, each of the one or more illumination beams are reflected by a mirror coupled to the substrate prior to illuminating the substrate.

In some embodiments, each of the one or more illumination beams illuminates a same sized field of view on the region of the substrate. In some embodiments, the field of view comprises an area at least 10 μm×10 μm, 10 μm×100 μm, 100 μm×100 μm, 10 μm×1 mm, 100 μm×1 mm, 1 mm×1 mm, 10 μm×10 mm, 100 μm×10 mm, 1 mm×10 mm, or 10 mm×10 mm. In some embodiments, the field of view comprises 2.6 mm×10 μm.

In some embodiments, the optical transformation device comprises one or more components selected from the group consisting of a micro-lens array (MLA), a diffractive optical element, a digital micro-mirror device (DMD), a phase mask, an amplitude mask, a spatial light modulator (SLM), and a pinhole array.

In some embodiments, the method further comprises providing initial illumination from a radiation source, wherein the initial illumination is directed through an additional optical transformation device to produce the one or more illumination beams. In some embodiments, the additional optical transformation device comprises one or more components selected from the group consisting of a micro-lens array (MLA), a diffractive optical element, a digital micro-mirror device (DMD), a phase mask, an amplitude mask, a spatial light modulator (SLM), and a pinhole array.

In some embodiments, illuminating the region of the substrate further comprises providing an additional illumination beam that is directed through the objective lens to the region of the substrate. In some embodiments, the additional illumination beam is provided from the radiation source. In some embodiments, the additional illumination beam is provided by directing the initial illumination through the optical transformation device. In some embodiments, the additional illumination beam is directed through the center of a diffraction plane of the objective lens.

In some embodiments, the emission light is directed through an additional optical transformation device prior to being received by the detector.

In some embodiments, each of the one or more illumination beams are directed to the region of the substrate at a respective angle relative to the objective lens. In some embodiments, the one or more illumination beams comprise 1, 2, 3, 4, 5, 6, 7, 8, 9, or 10 beams.

In some embodiments, the detector comprises one or more image sensors configured for time delay and integration imaging.

In some embodiments, the one or more illumination beams comprise an illumination pattern on the region of the substrate, wherein the illumination pattern comprises a plurality of light intensity maxima. In some embodiments, the illumination pattern comprises an interference pattern. In some embodiments, the illumination pattern is uniform within the region of the substrate. In some embodiments, the illumination pattern is hexagonal. In some embodiments, the illumination pattern is not uniform within the region of the substrate.

In some embodiments, the method further comprises repeating b) and c) at a plurality of time points, thereby generating a plurality of scanned images of the substrate.

In some embodiments, the scanned image exhibits a lateral spatial resolution that exceeds a diffraction-limited spatial resolution. In some embodiments, the scanned image exhibits a lateral spatial resolution improvement by a factor of better than 1.2, 1.5, 2, 2.5, 3, 3.5, 4, 4.5, 5, 5.5, or 6 times relative to an image obtained by a comparable diffraction-limited imaging system.

In some embodiments, the scanned image comprises a fluorescence image.

In some embodiments, the region of the substrate comprises an analyte and the emission light comprises light reflected, transmitted, scattered, or emitted by the analyte. In some embodiments, the analyte comprises a biological molecule. In some embodiments, the biological molecule comprises a nucleic acid molecule, a protein, a cell, or a tissue sample.

In some embodiments, the emission light corresponds to incorporation or a lack of incorporation of a nucleic acid base into a primer hybridized to the nucleic acid. In some embodiments, the emission light corresponds to incorporation of more than one nucleic acid base into the primer.

In some embodiments, repeating b) and c) determines a sequence of the nucleic acid.

In some embodiments, the substrate comprises a flow cell or surface for performing nucleic acid sequencing.

In another aspect, an imaging system is provided where the imaging system comprises: a) a substrate, wherein the substrate is substantially planar; b) a projection unit that is configured to i) direct illumination light onto a region of the substrate in an illumination pattern, wherein at least some of the illumination light is not directed through an objective lens, and ii) direct emission light from the substrate to one or more sensors via an optical transformation device, wherein the one or more sensors are configured for time delay and integration imaging; and c) one or more processors that are singly or collectively configured to perform the methods of claims 1-38.

In some embodiments, the optical transformation device comprises one or more components selected from the group consisting of a micro-lens array (MLA), a diffractive optical element, a digital micro-mirror device (DMD), a phase mask, an amplitude mask, a spatial light modulator (SLM), and a pinhole array.

In some embodiments, the emission light comprises light reflected, transmitted, scattered, or emitted by an analyte, wherein the analyte is positioned adjacent to the substrate.

In some embodiments, the projection unit is further configured to transmit illumination light through an additional optical transformation device to generate one or more illumination beams, wherein the one or more illumination beams provide the illumination pattern on the region of the substrate. In some embodiments, the additional optical transformation device comprises one or more components selected from the group consisting of a micro-lens array (MLA), a diffractive optical element, a digital micro-mirror device (DMD), a phase mask, an amplitude mask, a spatial light modulator (SLM), and a pinhole array.

In some embodiments, the illumination pattern is uniform within the region of the substrate.

In some embodiments, the one or more illumination beams are each transmitted through liquid immersion couplers prior to illuminating the region of the substrate.

In some embodiments, the optical transformation device and the additional optical transformation device comprise a plurality of harmonically modulated phase masks or harmonically modulated amplitude masks with different orientations. In some embodiments, a spatial frequency and orientation of the optical transformation device matches that of the additional optical transformation device. In some embodiments, the optical transformation device and the additional optical transformation device comprises harmonically modulated phase masks, and wherein the optical transformation device is phase shifted relative to the additional optical transformation device.

In some embodiments, the one or more sensors comprise one or more time delay and integration (TDI) cameras, charge-coupled device (CCD) cameras, complementary metal-oxide semiconductor (CMOS) cameras, or a single-photon avalanche diode (SPAD) array.

In some embodiments, the projection unit is configured to provide illumination light at two or more excitation wavelengths.

In some embodiments, the one or more sensors are configured to detect fluorescence at two or more emission wavelengths.

As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example block diagram of an optical transform imaging system 100, in accordance with some embodiments.

FIG. 2 illustrates an example schematic of an optical transform imaging system 200 with a radiation source configured in a reflection geometry coupled to a tube lens directing the radiative energy to a projection unit, in accordance with some embodiments. The detection unit of the imaging system is shown with a single optical relay coupling the radiative energy reflected, scattered, or emitted by the object and received from the projection module to the image sensor, in accordance with some embodiments.

FIGS. 3A and 3B illustrate example schematics of optical transform imaging systems 300 with a radiation source configured in a transmission geometry sharing a tube lens with the system's detection unit. In FIG. 3A, a second optical transformation device 308 is included in the detection unit 313. In FIG. 3B a second optical transformation device 318 is instead included in the projection unit 312. The detection unit is shown collecting reflected radiative energy from the object in a reflection geometry with a relay lens, in accordance with some embodiments.

FIG. 4 illustrates an example schematic of an optical transform imaging system 400 with a radiation source configured in a reflection geometry coupled to a tube lens directing the radiative energy to a projection unit, in accordance with some embodiments.

FIG. 5 illustrates an example optical schematic of an optical transform imaging system 500 with a radiation source in a transmission geometry optically coupled to a tube lens directing the radiation source into a projection unit. The detection unit 512 of the imaging system is represented by a tube lens 507 coupling the reflected, scattered, or emitted radiative energy from the object to a second optical transformation device 508 disposed adjacent to a sensor, in accordance with some embodiments.

FIGS. 6A-6E illustrate features of example optical transform imaging systems, in accordance with some embodiments. FIG. 6A shows the illumination intensity emitted from a point source as recorded by a single centered pixel in a TDI imaging system. FIG. 6B shows illumination intensity emitted from the point source as recorded by several individual pixels of the TDI imaging system, including off-axis pixels. FIG. 6C shows an example schematic of an imaging system with an optical transformation device that rescales an image located at a first image plane of an object (e.g., a point emission source) and relays it to a second image plane. FIG. 6D provides a conceptual example of how pixels in a conventional TDI imaging system (left) and a single, centered pixel in a confocal TDI imaging system (e.g., using a single, aligned pinhole to block all other image sensor pixels from receiving light) (right) will record illumination intensity emitted from a point source. FIG. 6E provides a conceptual example of the illumination intensities recorded by multiple pixels (including off-axis pixels) in a TDI imaging system, and the impact of using an optical transformation device to redirect and redistribute photons on the effective point spread function of the imaging system.

FIGS. 7A-7C illustrate the geometry and form factor of one non-limiting example of an optical transformation device used in accordance with some implementations of the disclosed imaging system. Specifically, FIG. 7A and FIG. 7B illustrate exemplary micro-lens array (MLA) optical transformation devices comprising a staggered and/or tilted repeat pattern of micro-lenses, respectively, in accordance with some embodiments. FIG. 7C shows the MLA embedded in a reflective mask that may be placed in the optical path of the optical transform imaging system to generate an illumination light pattern, in accordance with some embodiments.

FIG. 8A illustrates an example of patterned illumination generated by an optical transform imaging system. FIG. 8B illustrates the correspondingly even distribution of spatially integrated intensity across a scanned object, in accordance with some implementations of the disclosed imaging systems.

FIGS. 9A-9B illustrate exemplary scan intensity data generated by an optical transform imaging system without a second optical transformation device (FIG. 9A) and with a second optical transformation device (FIG. 9B) incorporated into the imaging system, in accordance with some embodiments.

FIGS. 10A-10C illustrate examples of illumination light patterns generated by an optical transform imaging system and the corresponding scanning direction of the imaging system to acquire image data of an object, in accordance with some implementations. Specifically, FIG. 10A illustrates a staggered illumination light pattern generated by a micro-lens array optical transformation device in a multi-array configuration. FIGS. 10B and 10C illustrate a non-limiting example of a line-shaped pattern illumination array with respect to the scanning direction of the optical transform imaging system (with FIG. 10C illustrating stacking of multiple illumination arrays).

FIG. 11 illustrates an example schematic of the excitation optical path in an optical transform imaging system with a radiation source configured in a reflection geometry, in accordance with some implementations of the disclosed imaging systems. In FIG. 11, the pathway of illumination light 1104 provided by radiation source 1102 is shown.

FIGS. 12A-12B illustrate example schematics of an optical transform imaging system with a radiation source configured in a reflection geometry corresponding to the example shown in FIG. 11. FIG. 12A illustrates the emission optical pathway for a system comprising one micro-lens array (e.g., 1110) in the illumination pathway that produces an illumination light pattern 1112 (shown in FIG. 11) to illuminate object 1122. FIG. 12B illustrates an example with two micro-lens arrays (e.g., a first micro-lens array 1110 in the illumination pathway, and a second micro-lens array 1220 in the emission pathway).

FIG. 13 provides a flowchart illustrating an example method of imaging an object, in accordance with some implementations described herein.

FIG. 14 provides an example of the resolution improvement provided by optical transform TDI imaging systems, in accordance with some implementations described herein.

FIG. 15 illustrates the relationship between signal and resolution in different imaging methods.

FIG. 16 provides a non-limiting schematic illustration of a computing device in accordance with one or more examples of the disclosure.

FIGS. 17A-17D provide non-limiting examples of optical design strategies that may be used to implement photon reassignment for resolution improvement. FIG. 17A: non-descanning optical design for use with digital approaches to photon reassignment. FIG. 17B: descanning optical design for use with digital approaches to photon reassignment. FIG. 17C: rescanning optical design for implementing optical photon reassignment. FIG. 17D: alternative rescanning optical design for implementing optical photon reassignment.

FIGS. 18A-18E provide non-limiting examples of the pattern of illumination light projected onto the sample plane (FIG. 18B), the phase pattern for a first micro-lens array (MLA1) (FIG. 18C), the phase pattern for a second micro-lens array (MLA2) (FIG. 18D), and the pattern of illumination light projected onto the pupil plane (FIG. 18E), respectively, for the CoSI microscope depicted in FIG. 18A.

FIG. 18F provides a schematic illustration of the use of a micro-lens array in combination with a TDI camera to enable photon reassignment while compensating for linear motion between a moving sample and the camera.

FIG. 18G and FIG. 18H provide non-limiting examples of plots of the normalized system PSF in the x and y directions for a confocal microscope and for a CoSI microscope, respectively.

FIGS. 19A-19C provide non-limiting examples of simulation results for system PSF for a CoSI microscope as described herein. FIG. 19A: non-limiting example of a plot of FWHM of the system PSF as a function of the zero-order power. FIG. 19B: non-limiting example of a plot of peak-to-mean intensity ratio of the illumination pattern as a function of the zero-order power. FIG. 19C: non-limiting example of a plot of FWHM of the system PSF as a function of both zero-order power and photon reassignment coefficient.

FIG. 20A provides a non-limiting example of simulated system PSFs for different values of the photon reassignment coefficient, a.

FIG. 20B provides a non-limiting example of a plot of the peak value of the normalized system PSF as a function of the photon reassignment coefficient, a.

FIG. 21A provides a non-limiting example plot of illumination uniformity as a function of the orientation of the MLA in a CoSI microscope.

FIG. 21B provides a non-limiting example of the illumination pattern (upper panel) and plot of averaged illumination intensity as a function for distance on the sample (lower panel) for an MLA orientation angle of 0.0 degrees.

FIG. 21C provides a non-limiting example of the illumination pattern (upper panel) and plot of averaged illumination intensity as a function for distance on the sample (lower panel) for an MLA orientation angle of 6.6 degrees.

FIGS. 22A-22B provide non-limiting examples of system PSF plots for different MLA orientation angles. FIG. 22A: orientation angle=6.6°. FIG. 22B: orientation angle=6.0°.

FIG. 23A provides a non-limiting example plot that illustrates the predicted impact of lateral displacement of MLA2 on system PSF (plotted as a 2D projection on the x-y plane) for MLAs having a 23 μm pitch.

FIG. 23B provides a non-limiting example plot of system PSF FWHM (in the x direction) as a function of the MLA2 displacement in the CoSI microscope shown in FIG. 18A.

FIGS. 24A-24C show non-limiting examples of tolerance analysis results for lateral resolution, system PSF, and normalized system PSF peak intensity as a function of the separation distance between a long focal length MLA2 and the camera sensor. FIG. 24A: plot of lateral resolution (system PSF FWHM averaged over x and y) as a function of separation distance error. FIG. 24B: plot of normalized peak intensity of the system PSF as a function of separation distance error. FIG. 24C: plots of the 2D system PSF as a function of the separation distance.

FIGS. 25A-25C show non-limiting examples of tolerance analysis results for lateral resolution, system PSF, and normalized system PSF peak intensity as a function of the separation distance between a short focal length MLA2 and the camera sensor. FIG. 25A: plot of lateral resolution (system PSF FWHM averaged over x and y) as a function of separation distance error. FIG. 25B: plot of normalized peak intensity of the system PSF as a function of separation distance error. FIG. 25C: plots of the 2D system PSF as a function of the separation distance.

FIG. 26A provides a non-limiting example plot of normalized power within a pinhole aperture of defined diameter as a function of the pinhole diameter.

FIG. 26B provides a non-limiting example plot of the power ratio within a pinhole aperture of defined diameter as a function of the pinhole diameter.

FIG. 26C provides a non-limiting example of system PSF plotted as a function of pinhole aperture diameter without gamma correction (Gamma=1).

FIG. 26D provides a non-limiting example of system PSF plotted as a function of pinhole aperture diameter with gamma correction (Gamma=0.4).

FIG. 27 shows a non-limiting schematic of a CoSI system.

FIG. 28 illustrates a non-limiting comparison of the resolution achievable by CoSI (upper panels) and widefield (lower panels) imaging.

FIGS. 29A and 29B provide non-limiting examples of resolution achievable by CoSI and wide field imaging. FIG. 29A shows plots of resolution achieved by CoSI (top plots) and wide field (lower plots) imaging. FIG. 29B illustrates example images obtained by CoSI (upper panels) and wide field (lower panels) imaging.

FIG. 30A illustrates a non-limiting example of TDI imaging of a rotating object.

FIGS. 30B and 30C illustrate non-limiting examples of magnification adjustment via objective tilting (FIG. 30B) and objective and tube lens tilting (FIG. 30C).

FIG. 30D illustrates a non-limiting example of variable magnification across a field-of-view (FOV).

FIG. 30E and FIG. 30F provide non-limiting examples that illustrate the creation of magnification gradients by adjusting the working distance of the optical system. FIG. 30E provides a plot of the calculated magnification as a function of the working distance displacement. FIG. 30F provides a plot of the calculated magnification as a function of the working distance displacement with the distance between the objective and tube lens reduced.

FIGS. 31A and 31B illustrate an example illumination system, in a front view and a top schematic representation.

FIGS. 32A and 32B illustrate an example illumination system, in a front view and a top schematic representation. As depicted, at least a portion of the input illumination bypasses the objective system.

FIGS. 33A and 33B illustrate an example system for back illumination, in a front view and a top schematic representation. As depicted, at least a portion of the input illumination bypasses the objective system.

FIGS. 34A and 34B illustrate an example system for back illumination, in a front view and a top schematic representation. As depicted, at least a portion of the input illumination bypasses the objective system.

FIGS. 35A and 35B provide non-limiting examples of resolution improvements (e.g., as indicated by FWHM) from CoSI and external CoSI (xCoSI). FIG. 35A illustrates a comparison between widefield, CoSI, and xCoSI resolution in a case where excitation and emission wavelengths and a photon reassignment coefficient are held constant. FIG. 35B illustrates the impact of photon reassignment coefficient on resolution (e.g., FWHM) in an xCoSI system.

FIG. 36 illustrates an exemplary imaging setup for scanning for use with imaging systems and methods described herein.

FIG. 37 illustrates an example workflow for processing a sample for sequencing.

FIGS. 38A and 38B illustrate multiplexed stations in a sequencing system.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Definitions: Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range irrespective of whether a specific numerical value or specific sub-range is expressly stated. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Ranges may be expressed herein as from “about” one particular value, and/or to “about” another particular value. The terms “about” and “approximately” shall generally mean an acceptable degree of error or variation for a given value or range of values, such as, for example, a degree of error or variation that is within 20 percent (%), within 15%, within 10%, or within 5% of a given value or range of values.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining whether an element is present or not (for example, detection). These terms can include quantitative, qualitative, or quantitative and qualitative determinations. Assessing can be relative or absolute. “Detecting the presence of” can include determining the amount of something present in addition to determining whether it is present or absent, depending on the context.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit the scope of the systems and methods disclosed herein but rather are meant to be exemplary.

Any systems, methods, software, compositions, and platforms described herein are modular and not limited to sequential steps. Accordingly, terms such as “first” and “second” do not necessarily imply priority, order of importance, or order of acts.

As used herein, the terms “biological sample” or “biological analyte” generally refer to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. For example, a sample of bodily fluid obtained by a puncture method and comprise blood and/or plasma. Such a sample may comprise cells and/or cell-free nucleic acid material. Alternatively, the sample may be obtained from any other source including but not limited to blood, sweat, hair follicle, buccal tissue, tears, menses, feces, or saliva. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively, or in addition, nucleic acid molecules may not be included within cells (e.g., cell-free nucleic acid molecules).

The term “analyte,” as used herein, generally refers to an object that is directly or indirectly analyzed during a process (e.g., a chemical process, an imaging process, etc.). An analyte may originate (and/or be derived) from a sample (e.g., a biological sample). For example, an analyte may be or comprise a molecule, a macromolecule (e.g., nucleic acid, carbohydrate, protein, lipid), a cell, a tissue or tissue sample, or any combination thereof. In addition, an analyte may be or comprise a synthetic version or variant of any of the above. Processing an analyte may comprise conducting a chemical reaction, biochemical reaction, enzymatic reaction, hybridization reaction, polymerization reaction, etc. (or a combination thereof) in the presence of or on the analyte. Processing an analyte may comprise physical and/or chemical manipulation of the analyte and detection thereof. An analyte may be indirectly or directly coupled to a substrate.

In a specific example, an analyte may comprise a nucleic acid, where the nucleic acid is derived or obtained from a biological sample (e.g., a cell, a tissue sample, etc.) and where the nucleic acid is immobilized a substrate. Processing such an analyte may comprise performing a sequencing reaction of the analyte and detecting the results of such a reaction (e.g., detecting the incorporation or lack thereof of one or more nucleic acids into a growing primer molecule that is hybridized to a template analyte). Such detection may comprise determining the presence of, amount of, change in, or absence of fluorescence (e.g., a fluorescent label, a Forster resonance energy transfer (FRET) interaction, etc.) or charge (e.g., a chemical charge).

As used herein, a “detector” refers to device capable of detecting or measuring a signal (e.g., a signal derived from analyte processing). In some cases, a detector may be an electronic device that is configured to detect electromagnetic radiation (e.g., radiation incident upon one or more components of the detector). A detector may comprise a single sensor or a plurality of sensors. A detector may detect one or more signals. Detection may comprise continuous area scanning. A continuous area scanning detector may comprise a time delay and integration (TDI) charge coupled device (CCD), Hybrid TDI, or complementary metal oxide semiconductor (CMOS), or pseudo TDI device.

The term “continuous area scanning,” as used herein, generally refers to area scanning in linear or non-linear paths such as rings, spirals, or arcs on a moving (e.g., rotating and/or translation) substrate using an optical imaging system and a detector. Continuous area scanning may comprise the use of an imaging array sensor capable of continuous integration over a scanning area in which the scanning is synchronized (e.g., electronically synchronized) to the image of an object in relative motion. For example, relative motion between the detector units and the substrate may refer to motion by the detector units, motion of the substrate, or both.

Continuous area scanning detectors may scan at the same rate for all image positions and therefore may not be able to operate at the correct scan rate for all imaged points in a curved (or arcuate or non-linear) scan. Therefore, the scan may be corrupted by velocity blur for imaged field points on an object moving at a velocity different than the scan velocity. Continuous rotational area scanning may comprise an optical detection system or method that makes algorithmic, optical, and/or electronic corrections to substantially compensate for this tangential velocity blur, thereby reducing this scanning aberration. In some cases, different sensors of the detector may be separately configured to compensate for differential velocity blur of separate segments of the substrate being scanned. For example, the compensation is accomplished algorithmically by using an image processing algorithm that deconvolves differential velocity blur at various image positions corresponding to different radii on a rotating substrate to compensate for differential velocity blur. In some cases, the camera or scanner may apply or use a blur to compensate for differential velocity blur.

As used herein, the term “scanning” refers to detection of signals (i.e., capturing images) during relative motion of the detector and the object. As used herein, the term “imaging” refers to processing (e.g., analyzing) or using images collected from scanning.

The terms “immersion lens” or “immersion optical lens,” as used herein, generally refer to an objective that is configured to be immersed or encased in a non-atmospheric environment (e.g., an immersion medium). An immersion lens typically has a higher numerical aperture (NA) than non-immersion lenses of the same magnification. A higher numerical aperture of a lens may be correlated with an increased refractive index of the immersion medium. In some cases, an immersion lens may be enclosed in an immersion jacket (e.g., to encompass immersion media).

The term “open substrate”, as used herein, generally refers to a substantially planar substrate in which a single active surface is physically accessible at any point from a direction normal to the substrate. Substantially planar may refer to planarity at a micrometer level or nanometer level. Alternatively, substantially planar may refer to planarity at less than a nanometer level or greater than a micrometer level (e.g., millimeter level). An open substrate may have a patterned or unpatterned surface. One or more analytes may be coupled to an open substrate (e.g., reparatory for processing the one or more analytes). Different processing operations on substrates (e.g., open substrates), scanning mechanisms, and optical detection systems are described in International Pub. No. WO2019/099886A1 and U.S. Pat. No. 10,852,528B1, each of which is entirely incorporated herein by reference.

The term “field-of-view” (FOV), as used herein, generally refers to the area on the sample or substrate that is optically mapped (or is mappable) to an active area of the detector (e.g., one or more active sensors of the detector). A FOV may be segmented into two or more regions, each of which can be electronically controlled to scan at a different rate. These scanning rates may be adjusted to the mean projected object velocity within each region. The regions may be optically defined using one or more beam splitters or one or more mirrors. The two or more regions may be directed to two or more detectors. The regions may be defined as segments of a single detector or as distinct sensors of a single detector.

As used herein, the term “focal plane” refers to any plane perpendicular to an optical axis of an optical device described herein, specifically to such a perpendicular plane comprising a focal point (e.g., a plane upon where illumination and/or emission light is focused). As used herein, the terms “object plane” or “sample plane” refer to a focal plane in or on the object being imaged. As used herein, the term “image plane” refers to a focal plane incident upon a detector. In general, an image plane is a magnification of the sample plane. As used herein, the term “pupil plane” generally refers to a focal plane located inside the objective of an optical device described herein. In particular, a pupil plane represents a fast Fourier transform (FFT) of the sample plane or image plane.

As used herein, the term “optical device” refers to a device comprising one, two, three, four, five, six, seven, eight, nine, ten, or more than ten optical elements or components (e.g., lenses, mirrors, prisms, beam-splitters, filters, diffraction gratings, apertures, etc., or any combination thereof).

As used herein, the term “optical transformation device” refers to an optical device used to apply an optical transformation to a beam of light (e.g., to affect a change in intensity, phase, wavelength, band-pass, polarization, ellipticity, spatial distribution, etc., or any combination thereof).

As used herein, the term “lossless” when applied to an optical device indicates that there is no significant loss of light intensity when a light beam passes through, or is reflected from, the optical device. For a lossless optical device, the intensity of the light transmitted or reflected by the optical device has at least 80%, 85%, 90%, 95%, 98%, or 99% of the intensity of the incident light.

The terms “support” or “substrate,” as used herein, generally refer to any solid or semi-solid article on which analytes or reagents, such as nucleic acid molecules, may be immobilized. Nucleic acid molecules may be synthesized, attached, ligated, or otherwise immobilized. Nucleic acid molecules may be immobilized on a substrate by any method including, but not limited to, physical adsorption, by ionic or covalent bond formation, or combinations thereof. An analyte or reagent (e.g., nucleic acid molecules) may be directly immobilized onto a substrate. An analyte or reagent may be indirectly immobilized onto a substrate, such as via one or more intermediary supports or substrates. In an example, an analyte (e.g., nucleic acid molecule) is immobilized to a bead (e.g., support or substrate) which bead is immobilized to a substrate. A substrate may be 2-dimensional (e.g., a planar 2D substrate) or 3-dimensional. In some cases, a substrate may be a component of a flow cell and/or may be included within or adapted to be received by a sequencing instrument. A substrate may include a polymer, a glass, or a metallic material. Examples of substrates include a membrane, a planar substrate, a microtiter plate, a bead (e.g., a magnetic bead), a filter, a test strip, a slide, a cover slip, and a test tube. A substrate may comprise organic polymers such as polystyrene, polyethylene, polypropylene, polyfluoroethylene, polyethyleneoxy, and polyacrylamide (e.g., polyacrylamide gel), as well as co-polymers and grafts thereof. A substrate may comprise latex or dextran. A substrate may also be inorganic, such as glass, silica, gold, controlled-pore-glass (CPG), or reverse-phase silica. The configuration of a support may be, for example, in the form of beads, spheres, particles, granules, a gel, a porous matrix, or a substrate. In some cases, a substrate may be a single solid or semi-solid article (e.g., a single particle), while in other cases a substrate may comprise a plurality of solid or semi-solid articles (e.g., a collection of particles). Substrates may be planar, substantially planar, or non-planar. Substrates may be porous or non-porous and may have swelling or non-swelling characteristics. A substrate may be shaped to comprise one or more wells, depressions, or other containers, vessels, features, or locations. A plurality of substrates may be configured in an array at various locations. A substrate may be addressable (e.g., for robotic delivery of reagents), or by detection approaches, such as scanning by laser illumination and confocal or deflective light gathering. For example, a substrate may be in optical and/or physical communication with a detector. Alternatively, a substrate may be physically separated from a detector by a distance. A substrate may be configured to rotate with respect to an axis. The axis may be an axis through the center of the substrate. The axis may be an off-center axis. The substrate may be configured to rotate at any useful velocity. The substrate may be configured to undergo a change in relative position with respect to a first longitudinal axis and/or a second longitudinal axis.

The term “bead,” as described herein, generally refers to a solid support, resin, gel (e.g., hydrogel), colloid, or particle of any shape and dimensions. A bead may comprise any suitable material such as glass or ceramic, one or more polymers, and/or metals. Examples of suitable polymers include, but are not limited to, nylon, polytetrafluoroethylene, polystyrene, polyacrylamide, agarose, cellulose, cellulose derivatives, or dextran. Examples of suitable metals include paramagnetic metals, such as iron. A bead may be magnetic or non-magnetic. For example, a bead may comprise one or more polymers bearing one or more magnetic labels. A magnetic bead may be manipulated (e.g., moved between locations or physically constrained to a given location, e.g., of a reaction vessel such as a flow cell chamber) using electromagnetic forces. A bead may have one or more different dimensions including a diameter. A dimension of the bead (e.g., the diameter of the bead) may be less than about 1 mm, 0.1 mm, 0.01 mm, 0.005 mm, 1 μm, 0.1 μm, 0.01 μm, 1 nm, or may range from about 1 nm to about 100 nm, from about 100 nm to about 1 μm, from about 1 μm to about 100 μm, or from about 1 mm to about 100 mm.

The section headings used herein are for organizational purposes only and are not to be construed as limiting the subject matter described.

### Enhanced Resolution or Super-Resolution Imaging

In recent years, many methods have been developed that overcome the diffraction limit by various physical mechanisms, such as stimulated emission depletion microscopy (STED), photo-activated localization microscopy (PALM), stochastic optical reconstruction microscopy (STORM), reversible saturable optical fluorescence transitions microscopy (RESOLFT), etc. However, these approaches generally have low throughput, precluding their use for high-speed imaging applications, and in many cases these methods require specialized fluorophores. A faster and more general framework allowing for a modest but significant increase in resolution is provided by methods utilizing patterned illumination, such as confocal microscopy, structured illumination microscopy (SIM), and image scanning microscopy (ISM).

Prior publications have described the combination of TDI with multi-focal confocal microscopy, where a large number of stationary illumination points are projected onto the specimen, and an array of pinholes is used to spatially filter the resulting image to create a confocal image in a single TDI pass. This approach enables high-throughput imaging with sub-diffraction limited resolution and requires no computational overhead. However, its significant drawback is the steep reduction of signal in view of the modest resolution increase achieved that is an inherent limitation of confocal microscopy. This drawback is resolved in image scanning microscopy (ISM) and structured illumination microscopy (SIM), but these two approaches require the acquisition of multiple images with subsequent computational reconstruction of a resolution-enhanced image, which significantly increases the imaging system complexity and dramatically decreases throughput compared to conventional TDI imaging.

Another imaging modality based on the use of patterned illumination is a variant of ISM where the final image is generated directly on the image sensor without computational overhead. These techniques are known variously as re-scan confocal microscopy, optical photon (or pixel) reassignment microscopy (OPRA), or instant SIM. In these techniques, a resolution improvement is achieved by optical rerouting of light emanating from the sample to an appropriate location in the image plane, either by an arrangement of scanning mirrors or using a modified spinning disk. However, while some of these techniques provide enhanced-resolution images at relatively high frame rates, they are mainly applicable to imaging of a small field of view and are not readily compatible with scanning imaging modalities such as TDI. Therefore, as noted above, there remains an unmet need for systems, devices, and methods for imaging that can bring together the high throughput and SNR of TDI imaging with the resolution enhancement attainable using patterned illumination.

The trade-offs between imaging speed, signal-to-noise ratios (SNR), image resolution are key considerations for many imaging applications (e.g., nucleic acid sequencing, small molecule or analyte detection, in-vitro cellular biological systems, synthetic and organic substrate analyses, etc.). In some cases, when optimizing an imaging system for a given attribute, others may be compromised. For example, current imaging systems and methods focused on improving imaging resolution beyond the diffraction limit (e.g., stimulated emission depletion microscopy (STED), photo-activated localization microscopy (PALM), stochastic optical reconstruction microscopy (STORM), reversible saturable optical fluorescence transitions microscopy (RESOLFT), etc.) are indeed capable of producing images that have image resolution that exceeds the diffraction limit, yet have low imaging throughputs (e.g., long image acquisition times and/or small fields-of-view) that limit their applicability in applications where high-speed imaging is required. The present disclosure presents systems and methods that can improve imaging speed, SNR, and image resolution simultaneously.

### Optical Transform Imaging Systems

Provided herein are imaging systems that combine optical photon reassignment microscopy (OPRA) with time delay and integration (TDI) imaging to enable high throughput, high signal to noise ratio (SNR) imaging while also providing enhanced image resolution by utilizing illumination light an NA exceed that of a traditional objective lens. Optical photon reassignment microscopy (OPRA) is an optical technique for achieving enhanced image resolution without the need for the computer-based methods often previously applied to methods such as image scanning microscopy (ISM) to computationally reassign the detected light intensity at any point in time to a corresponding most probable position of an emitter during a scan (Roth, et al. (2013), “Optical photon reassignment microscopy (OPRA)”, Optical Nanoscopy 2:5). OPRA is an improvement on ISM (discussed above). As with ISM, it is a method that does not reject light and is thus capable of generating high SNR images. However, it also does not require digital processing and only requires acquisition of a single image, which minimizes technical noise.

In TDI imaging, an image sensor (e.g., a time delay and integration (TDI) charge-coupled device (CCD)) is configured to capture images of moving objects without blurring by having multiple rows of photosensitive elements (pixels) which integrate and shift signals to an adjacent row of photosensitive elements synchronously with the motion of the image across the array of photosensitive elements. An image comprises a matrix of analog or digital signals corresponding to a numerical value of, e.g., photoelectric charge, accumulated in each image sensor pixel during exposure to light. During each clock cycle (typically from about 1 to 10 microseconds), the signal accumulated in each image sensor pixel is moved to an adjacent pixel (e.g., row by row in a “line shift” TDI sensor). The last row of pixels is connected to the readout electronics, and the rest of the image is shifted by one row. The motion of the object being imaged is synchronized with the clock cycle and image shifts so that each point in the object is imaged onto the same point in the image as it traverses the field of view (i.e., there is no motion blur). The image sensor (or TDI camera) is either continuously exposed, or line shifts may be alternated with exposure intervals. Each point in the image accumulates signal for N clock cycles, where N is the number of active pixel rows in the image sensor. The ability to integrate signals over the duration of a scan provides for high sensitivity imaging at low light levels.

The imaging systems described herein combine these techniques by using novel combinations of optical transformation devices (and other optical components) to create structured illumination patterns for imaging an object, to reroute and redistribute the light reflected, transmitted, scattered, or emitted by the object, and to project the rerouted and redistributed light onto one or more image sensors configured for TDI imaging. The combinations of OPRA and TDI disclosed herein allow the use of static optical transformation devices, which confers the advantages of: (i) being much simpler than exiting implementations of OPRA-like systems, and (ii) enabling a wide field-of-view and hence a very high imaging throughput (similar to or exceeding the throughput of conventional TDI systems). The disclosed imaging systems may be configured to perform fluorescence, reflection, transmission, dark field, phase contrast, differential interference contrast, two-photon, multi-photon, single molecule localization, or other types of imaging.

In some cases, the disclosed imaging systems may be standalone imaging systems. Alternatively, or in addition, in some instances the disclosed imaging systems, or component modules thereof, may be configured as an add-on to a pre-existing imaging system.

The disclosed imaging systems may be used to image any of a variety of objects or samples. For example, the object may be an organic or inorganic object, or combination thereof. An organic object may comprise cells, tissues, nucleic acids, nucleic acids conjugated onto beads, nucleic acids conjugated onto a surface, nucleic acids conjugated onto a support structure, proteins, small molecule analytes, a biological sample as described elsewhere herein, or any combination thereof. An object may comprise a substrate comprising one or more analytes (e.g., organic, inorganic) immobilized thereto. The object may comprise any substrate as described elsewhere herein, such as a planar or substantially planar substrate. The substrate may be a textured substrate, such as physically or chemically patterned substrate to distinguish at least one region from another region. The object may comprise a substrate comprising an array of individually addressable locations. An individually addressable location may correspond to a patterned or textured spot or region of the substrate. In some cases, an analyte or cluster of analytes (e.g., clonally amplified population of nucleic acid molecules, optionally immobilized to a bead) may be immobilized at an individually addressable location, such that the array of individually addressable locations comprises an array of analytes or clusters of analytes immobilized thereto. The imaging systems and methods described herein may be configured to spatially resolve optical signals, at high throughput, high SNR, and high resolution, between individual analytes or individual clusters of analytes within an array of analytes or clusters of analytes that are immobilized on a substrate. At any one point in time, when the object is illuminated not all of the individually addressable locations within the scanned FOV will emit optical (e.g., fluorescent) signals. That is, for a given time point, at least one individually addressable location on the object and within the illuminated FOV will not emit an optical signal (e.g., a fluorescent intensity).

In some instances, the disclosed imaging systems may be used with a nucleic acid sequencing platform, non-limiting examples of which are described in PCT International Patent Application Publication No. WO 2020/186243, which is incorporated by reference herein in its entirety. In some instances, the disclosed imaging systems may comprise components (especially multiple optical transformation elements), as described in PCT International Application No. PCT/US2022/77551, which is incorporated by reference herein in its entirety.

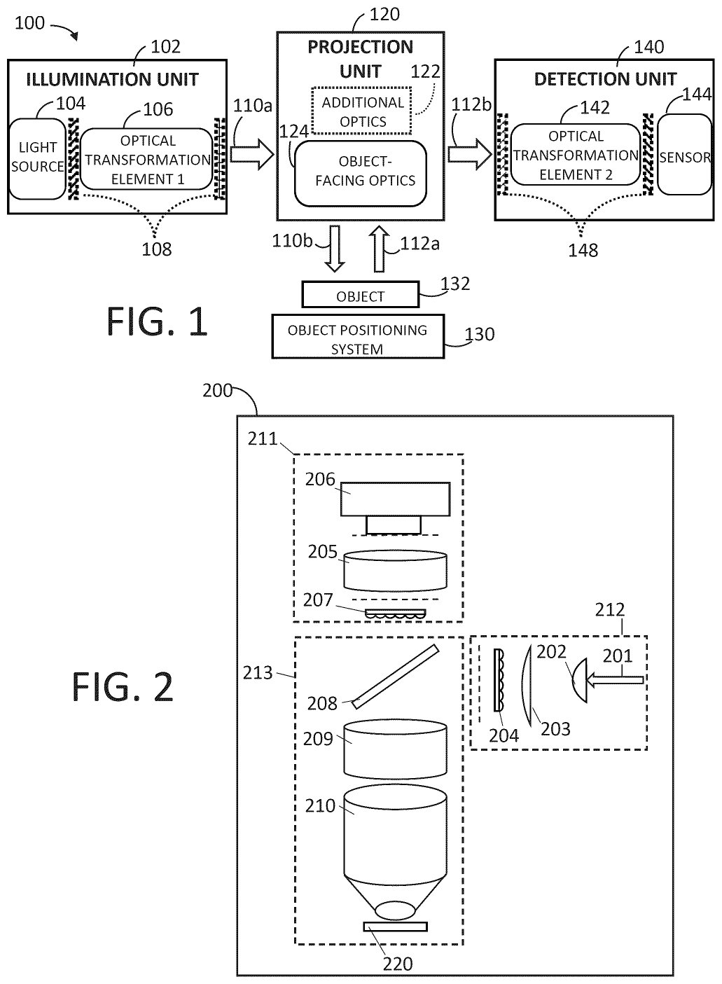

FIG. 1 provides a non-limiting example of an imaging system block diagram according to the present disclosure. In some cases, the imaging system 100 may comprise an illumination unit 102, projection unit 120, object positioning system 130, object 132, a detection unit 140, or any combination thereof. In some cases, the illumination unit 102, projection unit 120, object positioning system 130, and detection unit 140, or any combination thereof, may be housed as separate optical units or modules. In some cases, the illumination unit 102, projection unit 120, object positioning system 130, and detection unit 140 may be housed as a single optical unit or module.

In some instances, the illumination unit 102 may comprise a light source 104, a first optical transformation device 106, optional optics 108, or any combination thereof. In some instances, the light source (or radiation source) 104 may comprise a coherent source, a partially coherent source, an incoherent source, or any combination thereof. In some instances, the light source comprises a coherent source, and the coherent source may comprise a laser or a plurality of lasers. In some instances, the light source comprises an incoherent source, and the incoherent source may comprise a light emitting diode (LED), a laser driven light source (LDLS), an amplified spontaneous emission (ASE) source, a super luminescence light source, or any combination thereof.

In some instances, the first optical transformation device 106 is configured to apply an optical transformation (e.g., a spatial transformation) to a light beam received from light source 104 to create patterned illumination and may comprise one or more of a micro-lens array (MLA), diffractive element (e.g., a diffraction grating), digital micromirror device (DMD), phase mask, amplitude mask, spatial light modulator (SLM), pinhole array, or any combination thereof.

In some instances, the first optical transformation device comprises a plurality of optical elements that may generate an array of Bessel beamlets from a light beam produced by the light source or radiation source. In some instances, the first optical transformation device may comprise a plurality of individual elements that may generate the array of Bessel beamlets. The optical transformation device may comprise any other optical component configured to transform a source of light into an illumination pattern.

In some instances, the illumination pattern may comprise an array or plurality of intensity peaks that are non-overlapping. In some instances, the illumination pattern may comprise a plurality of two-dimensional illumination spots or shapes. In some instances, the illumination pattern may comprise a pattern in which the ratio of the spacing between illumination pattern intensity maxima and a full width at half maximum (FWHM) value of the corresponding intensity peaks is equal to a specified value. In some instances, for example, the ratio of the spacing between the illumination pattern intensity maxima and a full width at half maximum (FWHM) value of the corresponding intensity peaks may be 1, 2, 3, 4, 5, 10, 15, 20, 30, 40, 50, 60, 70, 80, 90, or 100.

In some cases, an uneven spacing between illumination spots or shapes may be generated by the optical transformation device to accommodate linear or non-linear motion of the object being imaged. In some instances, for example, non-linear motion may comprise circular motion. Various optical configurations and systems for continuously scanning a substrate using linear and non-linear patterns of relative motion between the optical system and the object (e.g., a substrate) are described in International Patent Pub. WO2020/186243, which is incorporated in its entirety herein by reference.

In some cases, the optional optics 108 of the illumination unit 102 may comprise one or more plano-convex lenses, bi-convex lenses, plano-concave lenses, bi-concave lenses, band-pass optical filters, low-pass optical filters, high-pass optical filters, notch-pass optical filters, quarter wave plates, half wave plates, or any combination thereof. In some instances, the illumination unit 102 is optically coupled with projection unit 120 such that patterned illumination 110a is directed to the projection unit.

In some instances, the projection unit 120 may comprise object-facing optics 124, additional optics 122, or any combination thereof. In some cases, the object-facing optics 124 may comprise a microscope objective lens, a plurality of microscope objective lenses, a lens array, or any combination thereof. In some instance the additional optics 122 of the projection unit 120 may comprise one or more dichroic mirrors, beam splitters, polarization sensitive beam splitters, plano-convex lenses, bi-convex lenses, plano-concave lenses, bi-concave lenses, band-pass optical filters, low-pass optical filters, high-pass optical filters, notch-pass optical filters, quarter wave plates, half wave plates, or any combination thereof. In some instances, the projection unit 120 is optically coupled to the object 132 such that patterned illumination light 110b is directed to the object 132, and light 112a that is reflected, transmitted, scattered, or emitted by the object 132 is directed back to the projection unit 120 and relayed 112b to the detection unit 140.

In some cases, the object positioning system 130 may comprise one or more actuators (e.g., a linear translational stage, two-dimensional translational stage, three-dimensional translational stage, circular rotation stage, or any combination thereof) configured to support and move the object 132 relative to the projection unit 120 (or vice versa). In some instances, the one or more actuators are optically, electrically, and/or mechanically coupled with (i) the optical assembly comprising the illumination unit 102, the projection unit 120, and the detection unit 140, or individual components thereof, and/or (ii) the object 132 being imaged, to effect relative motion between the object and the optical assembly or individual components thereof during scanning. In some cases, the object positioning system 130 may comprise a built-in encoder configured to relay the absolute or relative movement of the object positioning system 130, e.g., to a system controller (not shown) or the detection unit 140.

In some instances, the object 132 may comprise, for example, a biological sample, biological substrate, nucleic acids coupled to a substrate, biological analytes coupled to a substrate, synthetic analytes coupled to a substrate, or any combination thereof.

In some instances, the detection unit 140 may comprise a second optical transformation device 142, one or more image sensors 144 (e.g., 1, 2, 3, 4, or more than 4 image sensors), optional optics 148, or any combination thereof. In some cases, the second optical transformation 142 element may comprise a micro-lens array (MLA), diffractive element, digital micromirror device (DMD), phase mask, amplitude mask, spatial light modulator (SLM), pinhole array, or any combination thereof. In some cases, the one or more image sensors 144 may comprise a time delay integration (TDI) camera, charge-coupled device (CCD) camera, complementary metal-oxide semiconductor (CMOS) camera, or a single-photon avalanche diode (SPAD) array. In some instances, the time delay and integration circuitry may be integrated directly into the camera or image sensor. In some instances, the time delay and integration circuitry may be external to the camera or image sensor. In some instances, the optional optics 148 may comprise one or more plano-convex lenses, bi-convex lenses, plano-concave lenses, bi-concave lenses, band-pass optical filters, low-pass optical filters, high-pass optical filters, notch-pass optical filters, quarter wave plates, half wave plates, or any combination thereof.

As noted above, the illumination unit 102 may be optically coupled to the projection unit 120. In some instances, the illumination unit 102 may emit illumination light 110a that is received by the projection unit 120. The projection unit 120 may direct the illumination light 110b toward the object 132. The object may absorb, scatter, reflect, transmit (in other optical configurations), refract, or emit light (112a), or any combination thereof, upon interaction between the object 132 and the illumination light 110b. The light emanating from the object 112a directed towards the projection unit 120 may be directed 112b to the detection unit 140.

During operation, the projection unit 120 may direct an illumination pattern (received from the illumination unit 102) to the object 132 and receive and direct the resultant illumination pattern reflected, transmitted, scattered, emitted, or otherwise received from the object 132, also referred to herein as a “reflected illumination pattern” to the detection unit 140.

### Optical Transform Imaging System Configurations

The optical elements, and configuration thereof, of system 100 illustrated in FIG. 1 can be varied while still achieving high-throughput, high SNR, and enhanced resolution imaging. Variations of the optical system may share an optical path that, with or without additional optical elements (e.g., relay optics) at various stages, configures the light to travel from a radiation source (e.g., which is configured to output light) to a first optical transformation device to perform a first transformation to generate an illumination pattern, which illumination pattern is directed to an object, which object emits a reflected, transmitted, scattered, or emitted pattern of light (e.g., light output from the object or the object plane), which is then directed to a second optical transformation device to perform a second transformation to generate an image at one or more image sensors. Direction of illumination patterns to and from the object may be performed using any of a variety of configurations of the optical elements of the optical projection unit (e.g., a dichroic mirror and objective lens), as will be described below. In some instances, light is output from or by at least a portion of the object. Accordingly, an optical imaging system of the present disclosure may comprise at least a radiation source, a first optical transformation device, a second optical transformation device, and a detector.

Non-limiting examples of imaging system optical configurations that may perform high-throughput, high SNR imaging of an object with an enhanced resolution are illustrated in FIGS. 2-5. In some instances, the imaging system optical configurations may comprise alternative optical paths between: (i) the illumination unit (or pattern illumination source) optical assembly with respect to the projection unit (or pattern illumination projector) optical assembly, (ii) the projection unit optical assembly with respect to the detection unit optical assembly, or (iii) the illumination unit optical assembly with respect to the detection unit optical assembly. In some cases, the alternative optical paths may comprise alternative geometrical optical paths of the pattern illumination source, projection optical assembly, detection unit or any combination thereof. The alternative optical paths may comprise alternative collections of optical components and/or alternative ordering of such components in the pattern illumination source, projection optical assembly and detection unit. The alternative optical paths, in some instances, may provide advantages in terms of lower imaging system cost, complexity, robustness, tunability, modularity, or any combination thereof.

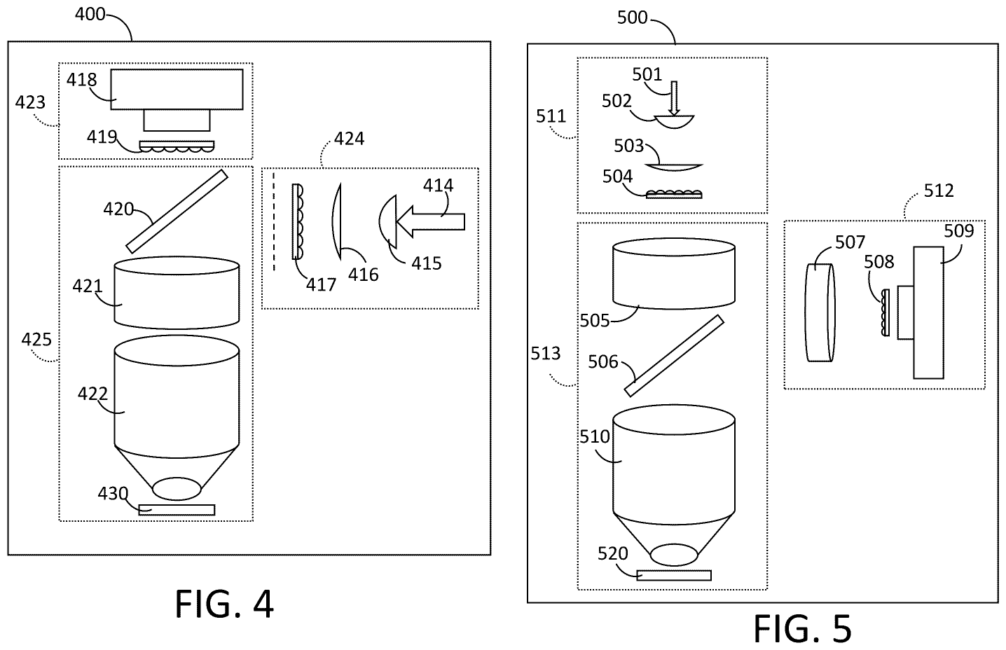

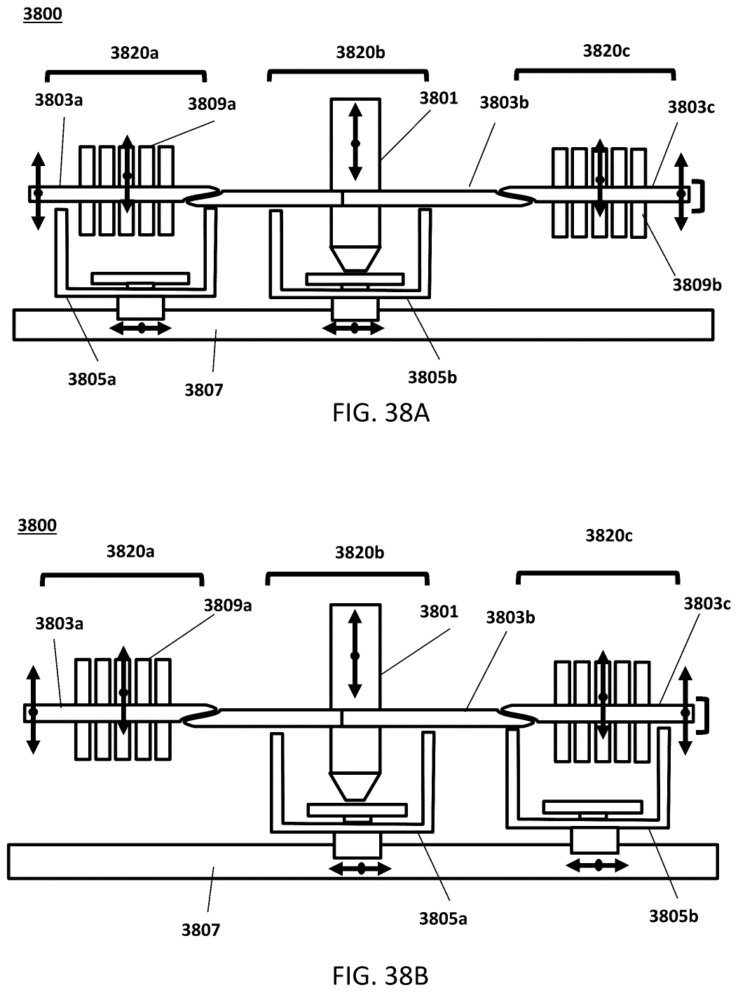

In some examples, the pattern illumination source may be in either a transmission optical geometry (see, e.g., FIGS. 38A, 38B, and 5) or a reflectance optical geometry (see, e.g., FIGS. 2 and 4) with respect to the projection optical assembly. In some instances, the dichroic mirror of the projection optical assembly may comprise a coated surface providing transmission or reflectance of light from the pattern illumination source dependent upon the optical geometry of the pattern illumination source with respect to the projection optical assembly.

FIG. 2 illustrates an example imaging system 200, according to the present disclosure that may comprise a pattern illumination source 212 in a reflection geometry with respect to the projection optical assembly 213. In some instances, the pattern illumination source 212 may comprise a radiation source 201, one, two, or more than two additional optical components (e.g., 202, 203), and a first optical transformation device 204. In some instances, the one, two, or more than two additional optical components (e.g., 202, 203) may be used to modify the beam shape or diameter of the input radiation 201. In some instances, the one or more additional optical elements may comprise plano-convex lenses, plano-concave lenses, bi-convex lenses, bi-concave lenses, positive meniscus lenses, negative meniscus lenses, axicon lenses, or any combination thereof. In some cases, the one or more optical elements may be configured to decrease or increase the diameter of the input radiation. Alternatively, the one or more optical elements may transform the input radiation beam shape into a Bessel, flat-top, or Gaussian beam shape. In addition, the one or more additional optical elements may be configured to cause the input radiation to converge, diverge, or form a collimated beam. In the example illustrated by FIG. 2, the optical elements 202 and 203 are two lenses configured as a Galilean beam expander to increase the initial input radiation's beam diameter to fill the field of view of the first optical transformation device 204. In some instances, the one or more additional optical elements may be configured to transform the intensity profile of the input radiation to any desired shape.

In some cases, the projection optical assembly 213 may comprise a first dichroic mirror 208, tube lenses 209, and an objective lens 210 which directs the patterned illumination to object 220. In some instances, the detection unit 211 may comprise a second optical transformation device 207, tube lens 205, and one or more sensors 206. In some instances, the tube lens 205 receives and directs the illumination pattern emitted or otherwise received from the object via the projection optical assembly 213 to the sensor 206. The tube lens 205 in combination with tube lens 209 of the projection optical assembly 213 may be configured to provide a higher magnification of the illumination pattern emitted or received from the object 220 and relayed to the sensor 206. In some instances, the one or more image sensors 206 of the detection unit 211 are configured for time delay and integration (TDI) imaging.

In some instances, imaging system 200 (or any of the other imaging system configurations described herein) may comprise an autofocus (AF) mechanism (not shown). An AF light beam may be configured to provide feedback to adjust the position of the objective lens with respect to the object being imaged, or vice versa. In some instances, the AF beam may be co-axial with the pattern illumination source 212 optical path. In some instances, the AF beam may be combined with the pattern illumination source using a second dichroic mirror (not shown) that reflects the AF beam and transmits the pattern illumination source radiation to the object being imaged.

In some instances, imaging system 200 (or any of the other imaging system configurations described herein) may comprise a controller. In some instances, a controller (or control module) may be configured, for example, as a synchronization unit that controls the synchronization of the relative movement between the imaging system (or the projection optical assembly) and the object with the time delay integration (TDI) of the one or more image sensors. In some instances, a controller may be configured to control components of the patterned illumination unit (e.g., light sources, spatial light modulators (SLMs), electronic shutters, etc.), the projection optical assembly, the patterned illumination detector (e.g., the one or more image sensors configured for TDI imaging, etc.), the object positioning system (e.g., the one or more actuators used to create relative motion between the object and the projection optical assembly), the image acquisition process, post-acquisition image processing, etc. In some instances, a galvo-mirror is used to scan all or a portion of the object (e.g., to enable TDI imaging). In some instances, the scanning performed by the galvo-mirror may be used to provide apparent relative motion between the object and the projection optical assembly.

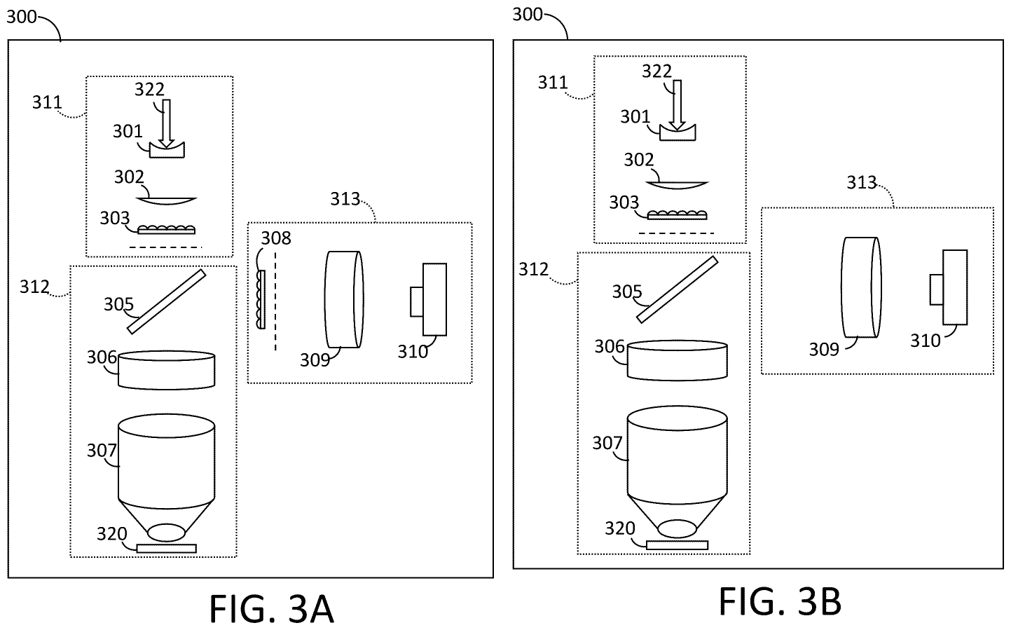

FIG. 3A illustrates an additional optical configuration for imaging system 300 where a patterned illumination source 311 is in a transmission geometry with respect to the projection unit 312 (e.g., projection optical assembly). In some instances, the pattern illumination source 311 may comprise a radiation source 322, plano-convex lenses (301, 302), and a first optical transformation device 303. In some instances, the projection optical assembly 312 may comprise a dichroic mirror 305, tube lens 306, and an objective lens 307 which directs an illumination pattern to object 320 and collects light reflected, scattered, or emitted therefrom. In some cases, the detection unit 313 may comprise a second optical transformation device 308, tube lens 309, and one or more image sensors 310. In some cases, the dichroic mirror 305, tube lens 306, and objective lens 307 of the projection optical assembly may be configured to both receive and direct the patterned illumination from pattern illumination source 311 to the objective lens 307, as well as to receive and direct the patterned light reflected, scattered, or emitted from the object to the detection unit 313. In some instances, the one or more image sensors 310 of the detection unit 313 are configured for time delay and integration (TDI) imaging.

FIG. 3B illustrates yet another optical configuration for imaging system 300 where a patterned illumination source 311 is in a transmission geometry with respect to the projection optical assembly 312. Again, the pattern illumination source 311 may comprise a radiation source 322, plano-convex lenses (301, 302), and a first optical transformation device 303. The projection optical assembly 312 may comprise a dichroic mirror 305, a second optical transformation device 318, tube lens 306, and an objective lens 307 which directs an illumination pattern to object 320 and collects light reflected, scattered, or emitted therefrom. In this configuration, the detection unit 313 comprises tube lens 309 and one or more image sensors 310, the second optical transformation device 318 having been moved to the projection optical assembly 312. In some instances, the one or more image sensors 310 of the detection unit 313 are configured for time delay and integration (TDI) imaging.

FIG. 4 illustrates an optical configuration for an imaging system 400 where a patterned illumination source 424 is in a reflection geometry with respect to the projection unit 425 (e.g., projection optical assembly). The imaging system 400 is further illustrated with a shared single tube lens 421 configured to couple the radiation source 414 to the projection unit 425 and to direct reflected, scattered, or emitted radiation energy to a detection unit 423 of the imaging system. The detection unit of the imaging system is shown coupling reflected, scattered, or emitted light from the shared single tube lens in the projection module to the second optical transform element 419 that is adjacent to the detection unit image sensor. In some instances, the pattern illumination source 424 may comprise a radiation source 414, plano-convex lenses (415, 416), and a first optical transformation device 417. In some cases, the projection unit 425 may comprise a dichroic mirror 420, tube lens 421, and an objective lens 422 configured to direct patterned illumination to object 430. In some instances, the detection unit 423 may comprise a second optical transformation device 419, and one or more image sensors 418. In some instances, the dichroic mirror 420, tube lens 421, and objective lens 422 of the projection unit 425 may be configured to both receive and direct the patterned illumination from pattern illumination source 424 to the object 430 being imaged as well as receive and direct the patterned light reflected, scattered, or emitted by the object to the detection unit 423. In some instances, the one or more image sensors 418 of the detection unit 423 are configured for time delay and integration (TDI) imaging.

FIG. 5 illustrates an example optical configuration for an imaging system 500 configuration where a patterned illumination source 511 is in a transmission geometry with respect to the projection optical assembly 513. In some cases, the pattern illumination source 511 may comprise a radiation source 501, plano-convex lenses (502,503), and a first optical transformation device 504. In some instances, the projection optical assembly 513 may comprise a dichroic mirror 506, tube lens 505, and an objective lens 510 configured to direct patterned illumination light to object 520. In some cases, the detection unit 512 may comprise a second optical transformation device 508, tube lens 507 and one or more image sensors 509. In some instances, the dichroic mirror 506, tube lens 505, and objective lens 510 of the projection optical assembly 513 may be configured to both receive and direct the patterned illumination from pattern illumination source 511 to the object 520 being imaged as well as receive and direct the patterned light reflected, scattered, or emitted by the object to the detection unit 512. In some instances, the one or more image sensors 509 of the detection unit 512 are configured for time delay and integration (TDI) imaging.

In some instances of any of the imaging system configurations described herein, one or both of the optical transformation devices (or optical transformation elements) may be tilted and/or rotated to allow collection of signal information in variable pixel sizes (e.g., to increase SNR, but at the possible of cost of increased analysis requirements). Tilting and/or rotating of one or both of the optical transformation elements may be performed to alleviate motion blur.

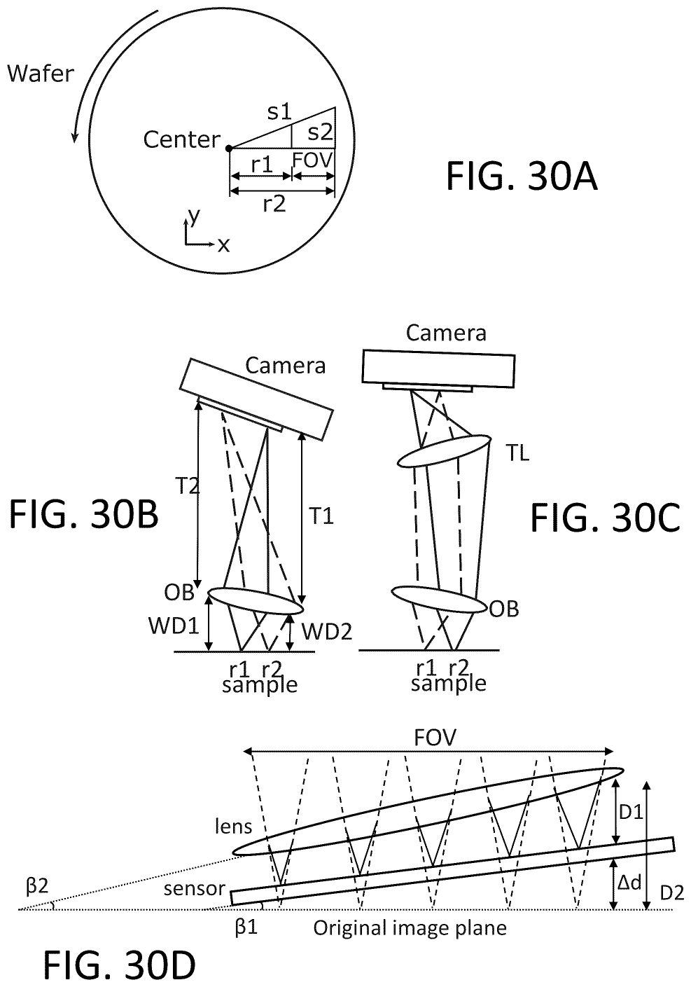

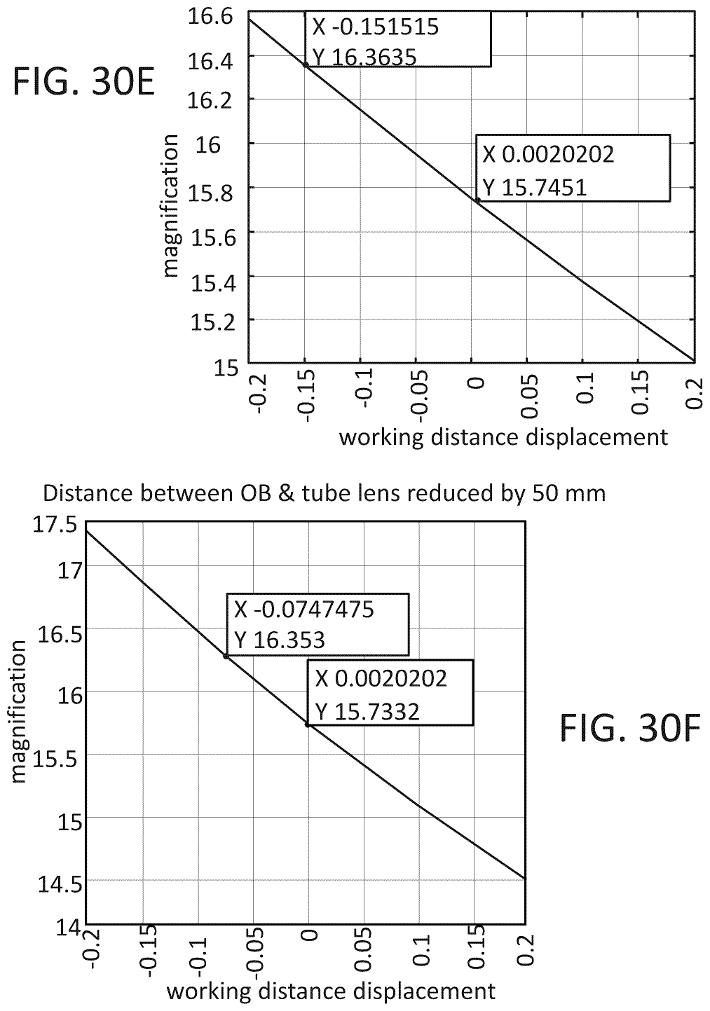

In some instances, motion blur may be caused by different linear velocities across the imaging system FOV, as illustrated in FIG. 30A. In such a case where the relative motion between the object and the imaging system comprises rotational motion centered about a rotational axis located outside the field-of-view of the imaging system, the main technical challenge is caused by the fact that at radius r₁ (corresponding to the innermost side of the image sensor) and at radius r₂ (corresponding to the outermost side of the image sensor), the object to be imaged, e.g., a rotating wafer, moves by different distances (S₁ and S₂, respectively) during the image acquisition time (see FIG. 30A). A TDI sensor can only move at a single speed, and thus can match the velocity of a circular object's movement at only one location in the sensor. This is typically optimized so that the center of the sensor matches the object's movement (e.g., the center, r₁/2+r₂/2). In CoSI, at the sample plane (e.g., at the surface of the wafer), the optimal imaging system will increase the density of illumination peaks and also increase the illumination width in the y axis, thus reducing the peak intensity of illumination while maintaining collected number of fluorescent photons on the detector. Thus, the value of S₂ and S₁, and also (S₂−S₁), can be increased.

One strategy to compensate for this relative motion is to separate the motion into linear (translational) and rotational motion components. An alternative strategy is to use wedged counter scanning where a magnification gradient can be created by, e.g., altering the working distance across the field-of-view of the image sensor (e.g., the camera). For example, a magnification gradient that is characterized by a magnification ratio (i.e., the ratio of magnification at the outer radius of the sensor to magnification at the inner radius of the sensor) given by Magnification Ratio=(S₂/S₁)=(r₂/r₁)=1+(FOV/r₁) where an FOV=r₂−r₁ could be used to compensate for the relative motion. As an example, if FOV is 2.6 mm and r₁=60 mm, then the Magnification Ratio between S₂ and S₁ is approximately 1.04.

For an imaging system with a typical Scheimpflug layout (see FIG. 30B), one can achieve different magnifications at r₁ and r₂ (as observed by the camera) by tilting the objective (OB). A modern microscope is typically infinity corrected, includes both an objective and a tube lens, and is more-or-less telecentric (i.e., having a more-or-less constant magnification regardless of an object's distance or location in the field-of-view). In FIG. 30B, the magnification at point r₁ on the object (e.g., the sample) is M₁=T₁/WD₁, while it is M₂=T₂/WD₂ at location r₂, (where WD is the working distance between the objective and the sample). By tilting the objective, we can adjust the ratio M2/M1. In an exemplary microscope, the working distance must be increased or decreased by around 0.1 mm in order to achieve 2.5% change of magnification (see Example 5 below). In some instances (see FIG. 30C), the Scheimpflug layout can be extended by including a tube lens (TL). In such cases, the distance between the objective (OB) and the tube lens (TL) can be intentionally increased or decreased to break the telecentricity and create a gradient of magnification across the field-of-view. As shown, one can achieve a 5% change of magnification across the field-of-view by using a reduced distance between the objective and tube lens and a 0.1 mm working distance displacement (see Example 5 below).

Another strategy to compensate for this relative motion is to insert a tilted lens before a tilted image sensor (see FIG. 30D). In FIG. 30D, D₁ is the distance between the tilted sensor and the tilted lens, D₂ is the distance between the tilted lens and the original image plane, and Δd is D₂−D₁. If the focal length of the lens is f, then the magnification across the tilted lens can be determined as:

$${α}^{′} = \frac{D 1}{D 2} = \frac{f}{f + D 2} ,$$

where α′ is similar to the concept of the photon reassignment coefficient, α.

If α′ is set to 1, then D₂ will be 0 (and hence Δd will be 0), meaning that the sensor and the lens would be superimposed. If D₂ is 0.04f, then α′ will be 1/1.04 and Δd will be 0.0015f. The relative change in magnification between one edge of the FOV and the other can be determined as:

$$\frac{1}{{a}^{′}} = \frac{f}{f + D 2} .$$

In some instances, the sensor and the lens are tilted at a same angle (and if so, there will be no variable magnification). In some instances, the sensor and the lens are tilted at different angles (e.g., β₁ and β₂, respectively). In some instances, β₁ may be at least about 0.1, 0.2, 0.3, 0.4, 0.5, 0.6, 0.7, 0.8, 0.9, 1, 2, 3, 4, 5, 6, 7, 8, 9, or 10 degrees. In some instances, β₂ may be at least about 1, 2, 3, 4, 5, 6, 7, 8, 9, 10, 15, or 20 degrees. Those of skill in the art will recognize that β₁ and β₂ each may be of any value within their respective ranges, e.g., about 0.54 degrees and about 11 degrees.

Accordingly, in some instances, the disclosed imaging systems may be configured to redirect light transmitted, reflected, or emitted by the object to one or more optical sensors (e.g., image sensors) through the use of a tiltable objective lens configured to deliver the substantially motion-invariant optical signal to the one or more optical sensors (e.g., image sensors). In some instances, the redirecting of light transmitted, reflected, or emitted by the object to the one or more optical sensors further comprises the use of a tiltable tube lens and/or a tiltable image sensor. In some instances, tiltable objectives, tube lenses, and/or image sensors may be actuated using, e.g., piezoelectric actuators.

In some instances, the tilt angles for the objective, tube lens, and/or image sensor used to create a magnification gradient across the field-of-view may be different when the image sensor is positioned at a different distance (e.g., a different radius) from the axis of rotation. In some instances, the tilt angles for the objective, tube lens, and/or image sensor may each independently range from about ±0.1 to about ±10 degrees. In some instances, the tilt angles for the objective, tube lens, and/or image sensor may each independently be at least ±0.1 degrees, ±0.2 degrees, ±0.4 degrees, ±0.6 degrees, ±0.8 degrees, ±1.0 degrees, ±2.0 degrees, ±3.0 degrees, ±4.0 degrees, ±5.0 degrees, ±6.0 degrees, ±7.0 degrees, ±8.0 degrees, ±9.0 degrees, or ±10.0 degrees. Those of skill in the art will recognize that the tilt angles may, independently, be of any value within this range, e.g., about ±1.15 degrees.

In some instances, the nominal distance between the objective and tube lens may range from about 150 mm to about 250 mm. In some instances, the nominal distance between the objective and the tube lens may be at least 150 mm, 160 mm, 170 mm, 180 mm, 190 mm, 200 mm, 210 mm, 220 mm, 230 mm, 240 mm, or 250 mm. Those of skill in the art will recognize that the nominal distance between the objective and the tube lens may be of any value within this range, e.g., about 219 mm. In some instances, the distance between the objective and tube lens may be increased or decreased from their nominal separation distance by at least about ±5 mm, ±10 mm, ±15 mm, ±20 mm, ±25 mm, ±30 mm, ±35 mm, ±40 mm, ±45 mm, ±50 mm, ±55 mm, ±60 mm, ±65 mm, ±70 mm, ±75 mm, or ±80 mm. Those of skill in the art will recognize that the distance between the objective and tube lens may be increased or decreased by any value within this range, e.g., about ±74 mm.

In some instances, the working distance may be increased or decreased by at least about ±0.01 mm, ±0.02 mm, ±0.03 mm, ±0.04 mm, ±0.05 mm, ±0.06 mm, ±0.07 mm, ±0.08 mm, ±0.09 mm, ±0.10 mm, ±0.20 mm, ±0.40 mm, ±0.60 mm, ±0.80 mm, ±1.00 mm, ±1.20 mm, ±1.40 mm, ±1.60 mm, ±1.80 mm, ±2.00 mm, ±2.20 mm, ±2.40 mm, ±2.60 mm, ±2.80 mm, or ±3.00 mm. Those of skill in the art will recognize that the working distance may be any value within this range, e.g., about ±1.91 mm.

In some instances, the change in magnification across the field-of-view may be at least about ±0.2%, ±0.4%, ±0.6%, ±0.8%, ±1.0%, ±1.2%, ±1.4%, ±1.6%, ±1.8%, ±2.0%, ±2.2%, ±2.4%, ±2.6%, ±2.8%, ±3.0%, ±3.2%, ±3.4%, ±3.6%, ±3.8%, ±4.0%, ±4.2%, ±4.4%, ±4.6%, ±4.8%, ±5.0%, ±5.2%, ±5.4%, ±5.6%, ±5.8%, or ±6.0%. Those of skill in the art will recognize that the change in magnification across the field-of-view may be of any value within this range, e.g., about ±0.96%.

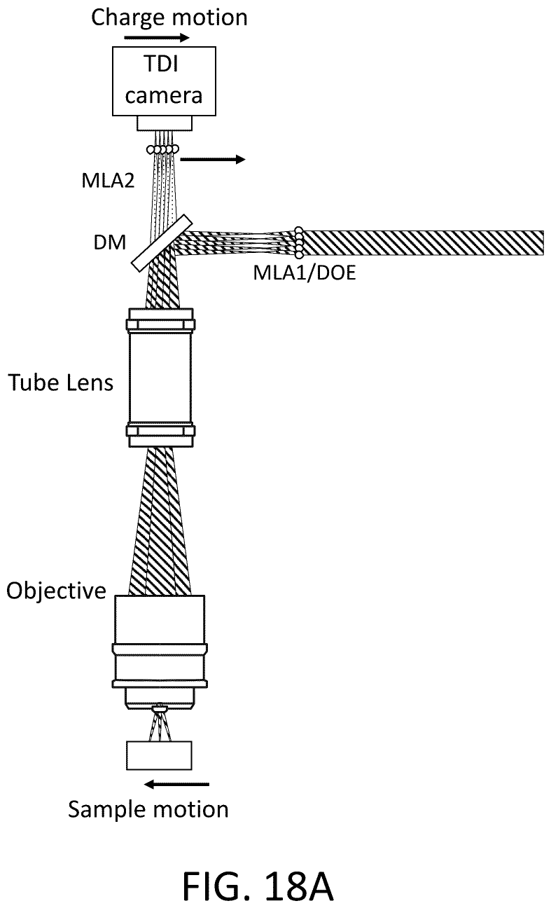

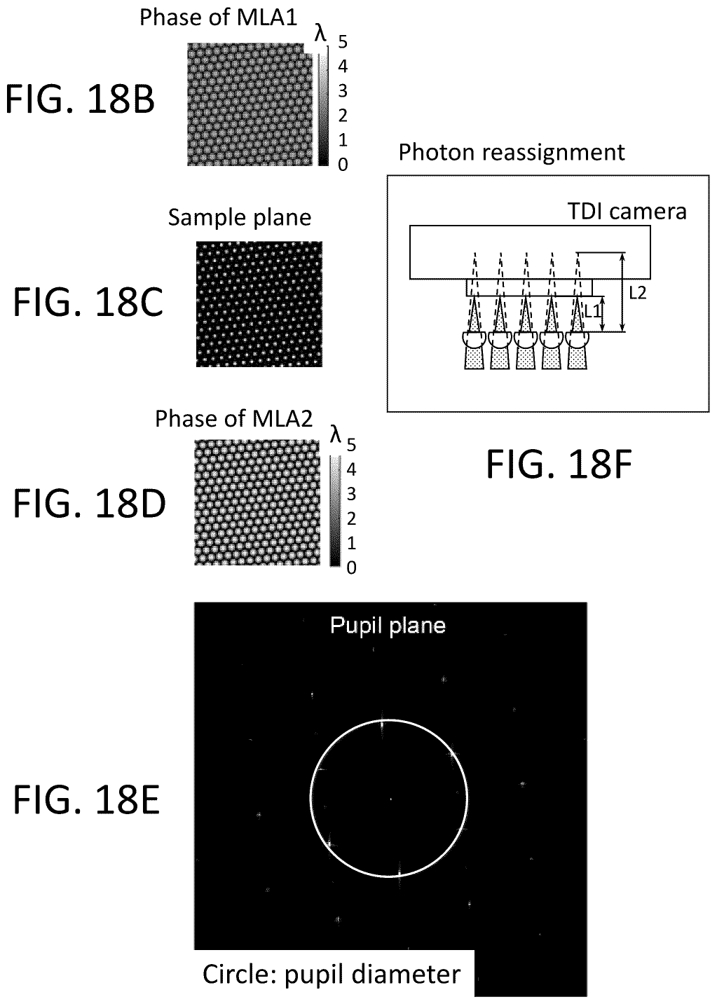

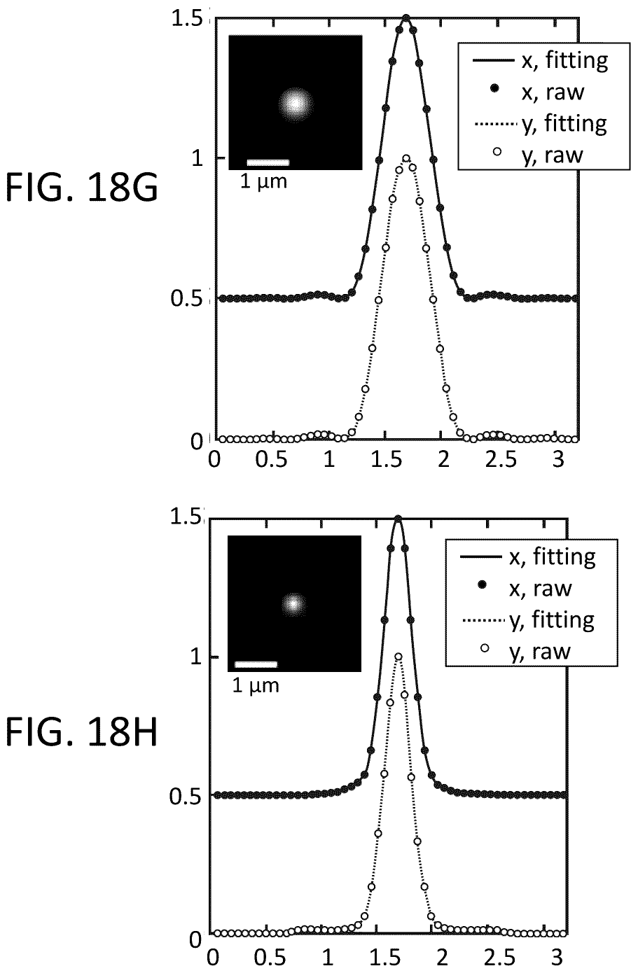

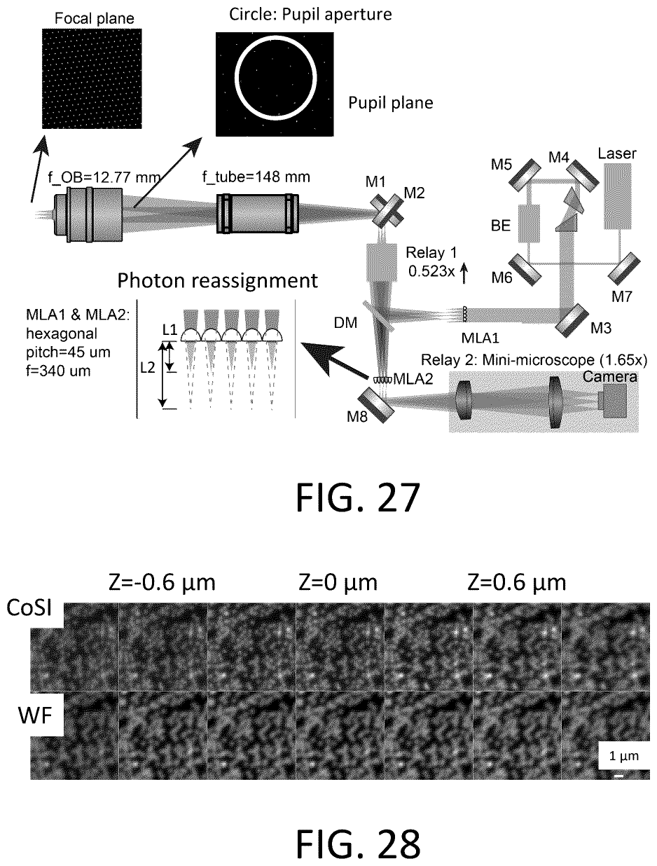

In some instances, of any of the imaging system configurations described herein, the position of the second optical transformation device (e.g., a second micro-lens array) may be varied. For example, in some instances, the second MLA may be positioned directly (e.g., mounted) on the image sensor. In some instances, the second MLA may be positioned on a translation stage or moveable mount so that its position relative to the image sensor (e.g., its separation distance from the sensor, or its lateral displacement relative to the sensor) may be adjusted. In some instances, the distance between the second MLA and the image sensor is less than 10 mm, 1 mm, 100 μm, 50 μm, 25 μm, 15 μm, 10 μm, 5 μm, or 1 μm or any value within a range therein. The location of the second MLA with respect to the sensor may be determined by the MLA's focal length (i.e., the second MLA may be positioned such the final photon reassignment coefficient is within a desired range). The photon reassignment coefficient is determined as the ratio of L1/L2, where L1 is the focal length of the second MLA and L2 is the effective distance of the second MLA2 to the sensor plane (see e.g., FIG. 18F or 27). For example, in some instances, the focal length of the second MLA is between 1 μm and 1000 μm, between 50 μm and 1000 μm, between 5 μm and 50 μm, or between 15 μm and 25 μm or any value within a range therein. For example, the second MLA may have a focal length of about 20 μm.

In some instances of any of the imaging system configurations described herein, the system may further comprise line-focusing optics for adjusting the width of a laser line used for illumination or excitation. For example, the line width of the focused laser may be made wider to reduce peak illumination intensity and avoid photodamage or heat damage of the object, while the line width of the focused laser line may be made narrower in order to reduce motion-induced blur). Photodamage is particularly problematic for objects comprising fluorophores (e.g., such as the fluorophores used in many sequencing applications).

### Patterned Illumination & Optical Transform Imaging

As illustrated in FIG. 1, the imaging systems disclosed herein may comprise an illumination unit (or pattern illumination source) 102 that provides light from a light source (or radiation source) 104 and optically transforms it using a first optical transformation device 106 to create patterned illumination that is focused on the object 132 to be imaged. A second optical transformation device 142 is used to apply a second optical transformation to the patterned light that is reflected, transmitted, scattered, or emitted (depending on the optical configuration of the imaging system and the imaging mode employed) from at least a portion of the object and relay the patterned light to one or more image sensors 144 that are configured for time delay and integration (TDI) imaging.

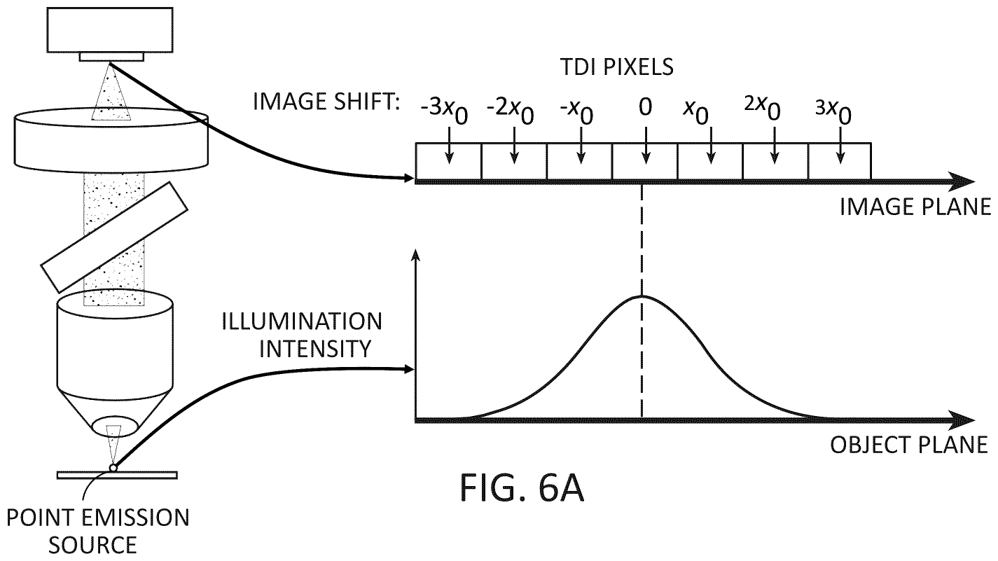

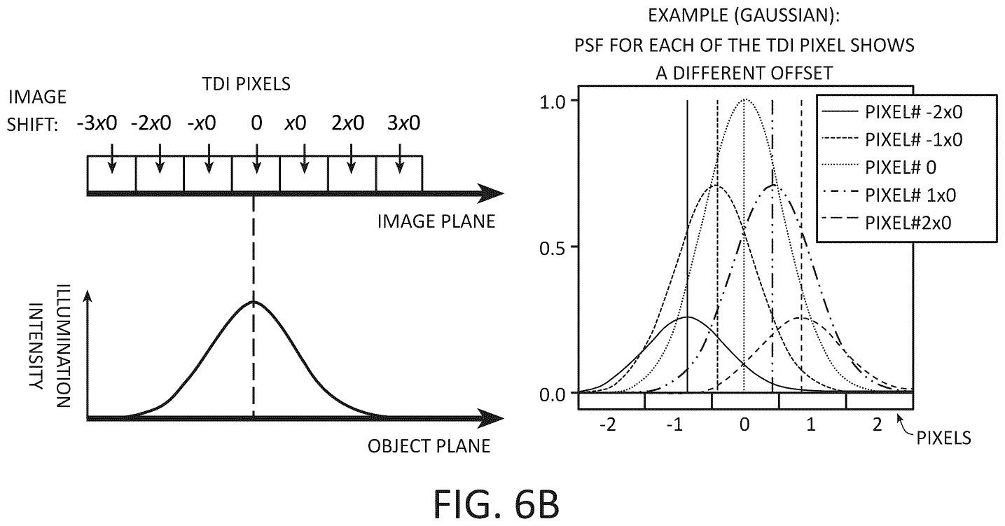

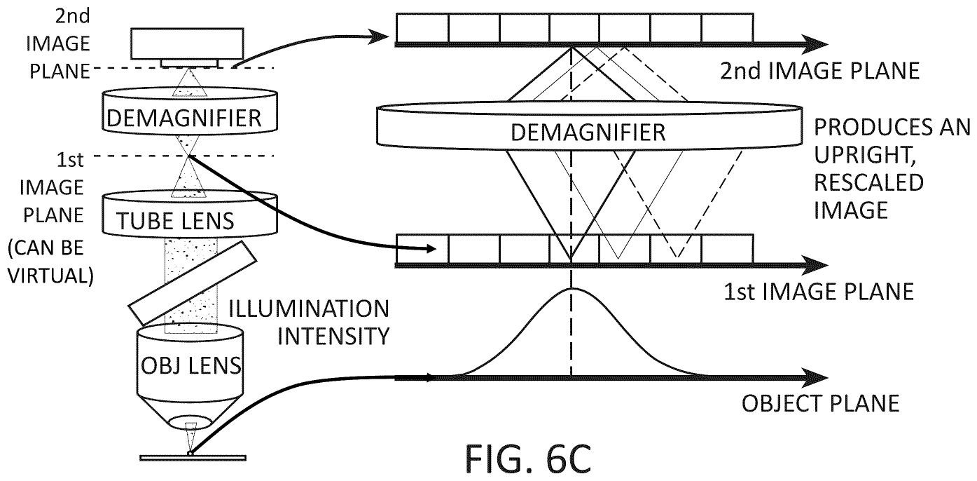

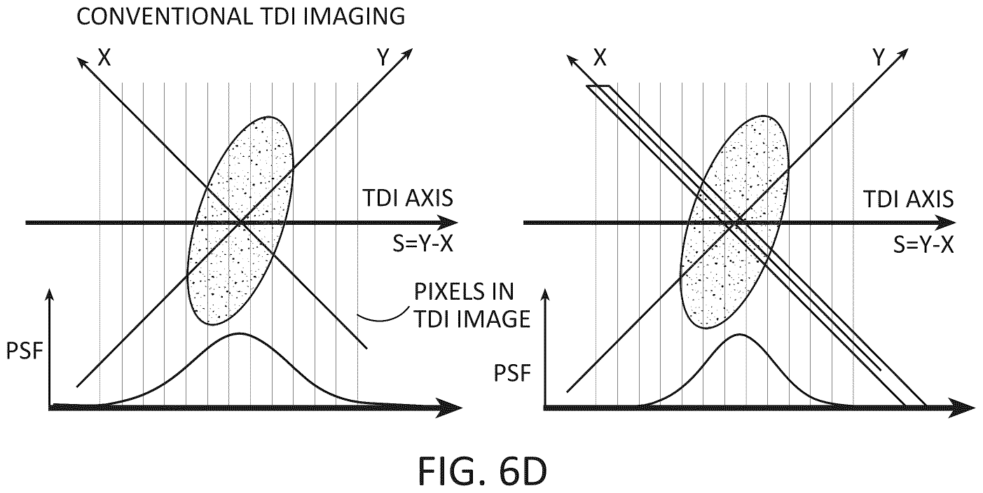

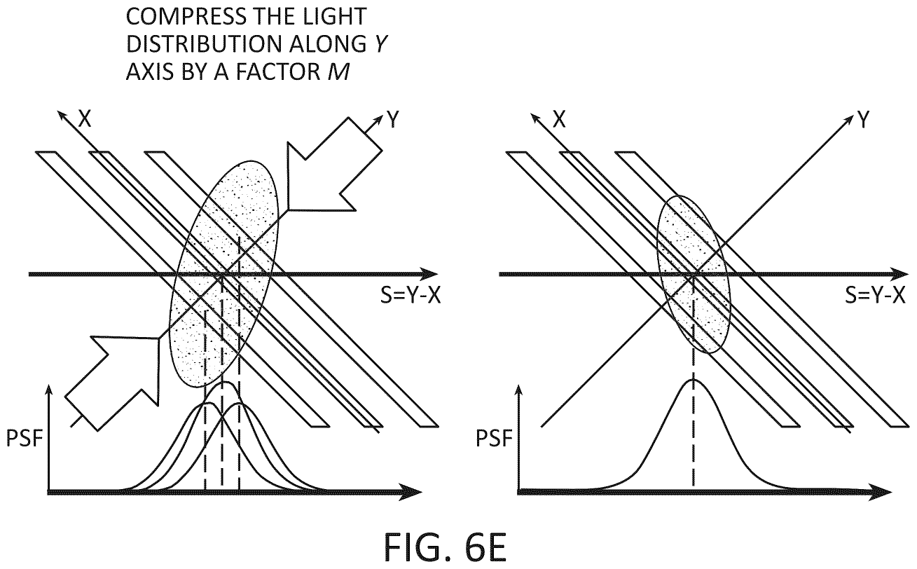

FIGS. 6A-6E illustrate features of the optical transform imaging systems described herein. FIG. 6A shows the illumination intensity emitted from a point source as recorded by a single pixel in a TDI imaging system (assuming that emitted light is blocked from reaching all other pixels, e.g., by using a single, aligned confocal pinhole in front of the image sensor). The illumination intensity peak of the recorded intensity profile coincides with the position of the single pixel at x=0 in the image plane. The width of the recorded illumination intensity profile is indicative of the point spread function (PSF) of the imaging system optics (i.e., a function that describes the response of the imaging system to a point source or point object). For off-axis pixels, the position of the illumination intensity peak in a TDI image is shifted relative to the position of the peak in the object plane by n x₀, where 0<x₀<a, a is the pixel size, and n is the pixel number (counting from the center position at x=0).

FIG. 6B shows illumination intensity emitted from the point source as recorded by several individual pixels of the TDI imaging system, including off-axis pixels. As noted above, the position of the intensity peak as recorded by off-axis pixels is shifted relative to the actual position of the peak in the object plane by a factor of n x₀. The examples of light intensity profiles illustrated for pixels at the −2xₒ, −xₒ, 0, +xₒ, and +2xₒ positions assume that the point spread function of the TDI imaging system is described by a Gaussian.

FIG. 6C shows an example schematic of a TDI imaging system (left) with an optical transformation device (e.g., a demagnifier) that rescales an image located at a first image plane of an object (e.g., a point source) and relays it to a second image plane located at an image sensor. As illustrated in FIG. 6C (right), the optical transformation unit (in this example, a demagnifying lens or lens array) collects the photons that are incident on the first image plane (which may comprise a virtual image), and redirects and redistributes them to form an upright, rescaled image (rescaled by a factor of M=1−(xₒ/δ)) at the second image plane that aligns and integrates the instantaneous “images” from individual pixels and corrects for the spatial shift, δ, in the positions of illumination intensity peaks that would otherwise be observed by the off-axis pixels of the image sensor.

FIG. 6D provides a conceptual example of how a TDI imaging system (left) with focused laser illumination using a single pinhole aligned with the illumination beam, blocking all image sensor pixels but one from receiving light (right), will record illumination intensity emitted from a point source. The vertical segments in each plot represent the pixels in the TDI image. X describes the position of the scan system, Y is the image-plane coordinate (the same as the sensor pixel coordinate in the scan direction), and S is the position of a pixel in the resulting TDI image (the images are one-dimensional in this simplified illustration). The plot shows relations between X, Y, and S, and the intensity distribution of an individual emitter as a function of those coordinates. The oval shape indicates the emitted light intensity I(x)=h(x)*g(y−x) recorded during a scan, where h(x) is the illumination point spread function, and g(x) is the detection point spread function. In the plot at right, the slanted region centered on y=0 and having boundaries at y=±a/2 represents the physical image sensor pixel at y=0. The intersection of the slanted region and the oval shape representing emitted light intensity is the fraction of emitted light that is allowed to reach the central image sensor pixel. For the confocal TDI imaging system, the resulting image intensity profile (right) has a narrower width (i.e., higher spatial resolution) than that for the conventional TDI imaging system (left) due to the use of the optical transformation device (i.e., the single, aligned pinhole in this non-limiting example) to prevent light from reaching the off-axis image sensor pixels.

FIG. 6E provides a conceptual example of the illumination intensities recorded by multiple pixels (including off-axis pixels) in a TDI imaging system, and the impact of using an optical transformation device to redirect and redistribute photons on the effective point spread function of the imaging system. The oval shape indicates the emitted light intensity I(x)=h(x)*g(y−x) recorded during a scan, where h(x) is the illumination point spread function, and g(x) is the detection point spread function. In the plot at left, the middle-slanted region centered on y=0 and having boundaries at y=±a/2 represents the physical image sensor pixel at y=0. The intersection of the middle-slanted region and the oval shape representing emitted light intensity is the fraction of emitted light that is allowed to reach the central image sensor pixel. Likewise, the two additional slanted regions represent two symmetrically placed off-axis pixels, and the intersection of the slanted regions and ovel shape representing emitted light intensity is the fraction of emitted light that reaches the two symmetrically placed, off-axis image sensor pixels. Each physical pixel collects a signal corresponding to light intensity profiles having a peak with a different spatial offset relative to the emission intensity peak in the object plane. Conventional TDI imaging systems accumulate (sum) signals from all physical pixels in the image sensor, resulting in deteriorated resolution. In the absence of a compensating optical transformation device, the image intensity profiles recorded by the three pixels are shifted relative to each other (bottom left) and sum to an overall profile that is broad compared to the individual profiles. Restricting light collection to just one physical pixel provides confocal resolution, but at the cost of losing most of the light and reducing the signal-to-noise ratio (SNR) of the image. A better solution is to compensate for the spatial shifts. If an optical transformation device is used (e.g., a demagnifying optical element that compresses the distribution of light along the Y axis by a factor of M=1−(xₒ/δ)), the light intensity profiles recorded by the three image sensor pixels are brought into alignment (lower right) and combine to form a recorded intensity profile that has a narrower width(i.e., a higher spatial resolution) than that obtained in the absence of the optical transformation device.

Mathematically, this optical compensation technique can be described in terms of a relative scaling of the point spread functions (PSFs) for the illumination and detection optics. The image intensity profile (or emitted light intensity profile) recorded during a scan is given by:

$$I ( x , y ) = h ( x ) \cdot g ( y - x )$$

where h(x) is the illumination PSF, and g(y−x) is the detection PSF. The optical transformation device used to compensate for the spatial shift between intensity peaks in the image and intensity peaks in the illumination profile at the object plane may, for example, apply a demagnification, m, in the Y direction such that y→my, where 0<m<1. The image intensity profile is then given by:

$$I ( x , y ) \to I ’ ( x , y ) = h ( x ) \cdot g ( my - x )$$

or, in terms of a scan coordinate, s=y−x:

$$PSF ( s ) ∝ \int {I}^{′} ( s , x ) dx$$

$$PSF ( s ) ∝ \int h ( x ) \cdot g ( \frac{s + ( 1 - m ) x}{m} ) dx = \int {h}^{′} ( z ) \cdot {g}^{′} ( s - z ) dz$$

where z=(1−m)x. More generally,

$$PSF ( s ) = [ {h}^{′} ⊗ {g}^{′} ] ( s ) , where {h}^{′} ( z ) = h ( ( - z ) / ( 1 - m ) ) {g}^{′} ( z / m ) = h ( z / m )$$

and ⊗ is the convolution operator. The PSF for the imaging system in this method is the convolution of the illumination point spread function, h, scaled by a factor (1−m) and the detection point spread function, g, scaled by a factor m. The PSF determines the resolution of the imaging system, and is comparable to, or better than, the point spread function (and image resolution) for a confocal imaging system (e.g., a diffraction limited conventional imaging system).

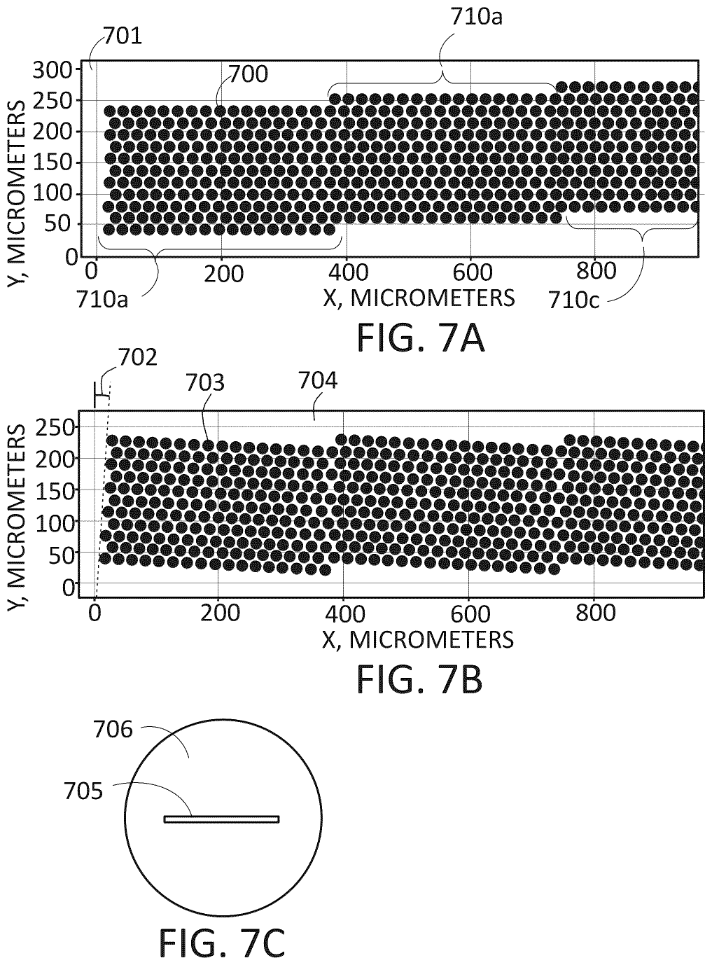

The same considerations apply when using structured illumination (patterned illumination) in combination with TDI imaging. In some cases, the pattern illumination source may comprise an optical transformation device used to generate structured illumination (or patterned illumination). FIGS. 7A-7C illustrate the geometry and form factor of one non-limiting example of an optical transformation device used in accordance with some implementations of the disclosed imaging system. FIG. 7A illustrates an exemplary micro-lens array (MLA) comprising a staggered rectangular repeat pattern 701 of individual micro-lenses 700 (e.g., where a row of the plurality of rows is staggered in the perpendicular direction with respect to an immediately adjacent previous row in the plurality of rows). In the non-limiting example of FIG. 7A, each rectangular repeat pattern comprises a plurality of micro-lenses in a hexagonal close packed configuration. In some instances, the plurality of micro-lenses in each repeat pattern may be packed in any regular or irregular packing configuration. In some instances, the regular arrangement of the plurality of micro-lenses is configured to provide equal spacing between adjacent micro-lenses. In some instances, the MLA may comprise multiple repeats of the rectangular pattern, e.g., 710a, 710b, and 710c, as shown in FIG. 7A, that are each offset (staggered) relative to the previous repeat by, for example, one row of micro-lenses, two rows of micro-lenses, three rows of micro-lenses, etc. In some instances, the rows and columns of micro-lenses may be aligned with, for example, the x and y coordinates of the rows and columns of pixels in a TDI image sensor such that the angle between a column of micro-lenses in the MLA device and a column of pixels is zero degrees.

FIG. 7B illustrates an exemplary micro-lens array (MLA) comprising a tilted rectangular repeat pattern 704 of individual micro-lenses 703. In the non-limiting example of FIG. 7B, each rectangular repeat pattern comprises a plurality of micro-lenses in a hexagonal close packed configuration. In some instances, the plurality of micro-lenses in each repeat pattern may be packed in any regular or irregular packing configuration. In some instances, the MLA may comprise multiple repeats of the rectangular pattern that are each tilted (or rotated) relative to, for example, the x and y coordinates of the rows and columns of pixels in a TDI image sensor such that the angle 702 between a column of micro-lenses in the MLA device and a column of pixels is a specified angle, e.g., from about 0.5 to 45 degrees.

FIG. 7C shows the MLA 705 embedded in a reflective mask 706 that may be placed in the optical path of the optical transform imaging system to generate an illumination light pattern, in accordance with some implementations of the disclosed imaging system. In some cases, the reflective mask may be comprised of chrome, aluminum, gold, silver, other metals or alloys, or any combination thereof.

In some examples, the plurality of micro-lenses may comprise a plurality of spherical micro-lenses, aspherical micro-lenses, or any combination thereof. In some instances, the MLA may comprise a plurality of micro-lenses with a positive or negative optical power. In some cases, the MLA may be configured such that the rows are aligned with respect to a scan or cross-scan direction. In some instances, the scan direction may be aligned with a length of the MLA defined by the number columns of micro-lenses. Alternatively, the cross-scan direction may be aligned with a width of the MLA defined by the number of rows of micro-lenses.

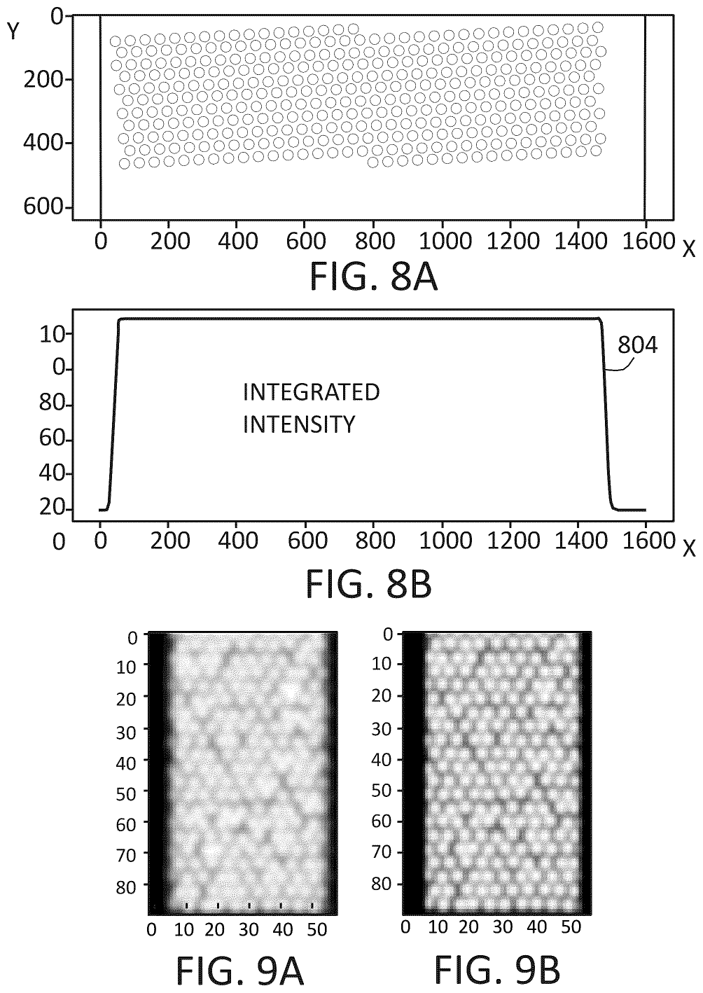

FIG. 8A illustrates an example of patterned illumination (x and y axis units in micrometers) generated by an optical transform imaging system comprising, e.g., a tilted, hexagonal pattern micro-lens array (where each row in the plurality of rows comprising the regular pattern is tilted), to produce patterned illumination. FIG. 8B illustrates the corresponding uniform distribution of spatially integrated illumination intensity 804 (in relative intensity units) across a scanned object (x axis units in micrometers), in accordance with some implementations of the disclosed imaging systems.

FIGS. 9A-9B illustrate exemplary scan intensity data as a function of pixel coordinates generated by an optical transform imaging system without a second optical transformation device (FIG. 9A) and with a second optical transformation device (FIG. 9B) incorporated into the imaging system to compensate for the spatial shift between intensity peaks in the image and the patterned illumination peaks in the object plane, in accordance with some implementations of the disclosed imaging systems. As can be seen, the resolution of the image in FIG. 9B is significantly improved compared to that obtained in FIG. 9A when no second optical transformation device was used.

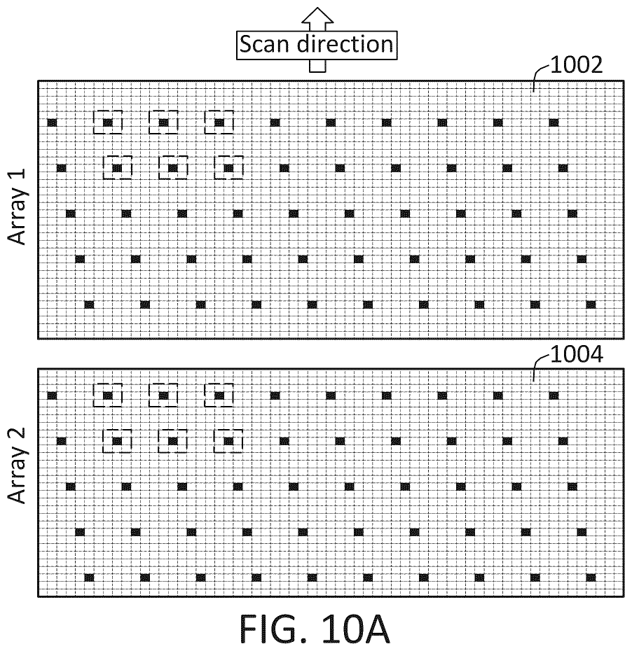

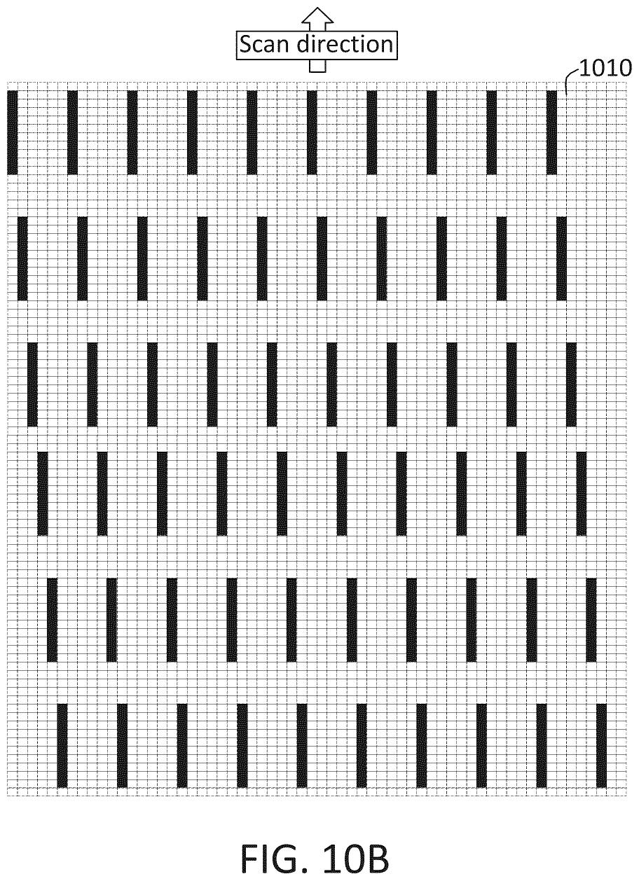

FIGS. 10A-10C illustrate examples of illumination light patterns generated by an optical transform imaging system and the corresponding scanning direction of the imaging system to acquire image data of an object, in accordance with some implementations of the disclosed imaging systems. Referring to FIG. 10A, an imaging system may be configured to scan an object in the indicated scan direction (upwards, as illustrated). The object may be, for example, a planar or substantially planar substrate. In some instances, the imaging system may generate and project a staggered array illumination pattern onto an object. The illumination pattern may comprise an array of non-overlapping illumination peaks. The illumination pattern may be selected such that each point in the object plane is illuminated by a series of illumination peaks. As translation occurs, either by moving the object or by moving the illumination pattern, or both, each intensity peak travels across the object plane along the scan direction axis. FIG. 10A illustrates a staggered illumination pattern generated by an optical transformation device comprising a micro-lens array in a multi-array configuration (e.g., array 1, 1002, array 2, 1004, etc.). A multi-array configuration may be used, for example, to ensure that the TDI image sensor is completely filled by the transformed light used to generate the image. In some instances, different arrays in a multi-array configuration may be used, for example, to create different illumination patterns, illumination patterns comprising different illumination wavelengths, and/or illumination patterns comprising different polarizations.

FIGS. 10B and 10C illustrate a non-limiting example of a line-shaped pattern illumination array 1010 aligned with respect to the scanning direction of the optical transform imaging system (with FIG. 10C illustrating stacking of multiple illumination arrays, i.e., the transformation elements comprise a sequence of sub-arrays with specific patterns).

The light pattern reflected, transmitted, scattered, or emitted by the object as a result of illumination by the patterned illumination (e.g., the “reflected light pattern”, “emitted light pattern”, etc.) is transformed (e.g., by the second optical transformation device) to create an intensity distribution representing a maximum-likelihood image of the object. In some cases, each point in the image plane may be represented by an intensity distribution that is substantially one-dimensional (1d) (i.e., the illumination pattern may consist of elongated illumination spots (line segments) that only confer a resolution advantage in the direction orthogonal to the line segments). In some cases, each point in the image plane may be re-routed to a different coordinate that represents the maximum-likelihood position of the corresponding emission coordinate on the object plane. In some cases, the light pattern emitted by the object and received at an image plane may be re-routed to form a two-dimensional (2d) intensity distribution that represents the maximum-likelihood 2d distribution of the corresponding emission coordinates on the object plane. In some instances, a series of illumination patterns may be used to create a larger illumination pattern that is used during a single scan. In some instances, a series of illumination patterns may be cycled through in a series of scans, and their signals and respective transformations accumulated, to generate a single enhanced resolution image. That is, the signal generated at each position and/or by each illumination pattern may be accumulated. In some cases, the illumination pattern may be selected such that each point of the object, when scanned through the field of view, receives substantially the same integral of illumination intensity over time (i.e., the same total illumination light exposure) as other points of the object (see, e.g., FIG. 8B).

In some examples, the imaging system may illuminate the object by an illumination pattern comprising regions of harmonically modulated intensity at the maximum frequency supported by the imaging system objective lens NA and illumination light wavelength. The pattern may consist of several regions with varying orientations of harmonically modulated light such that each illumination point in the illumination pattern directed to the object may be sequentially exposed to modulations in all directions on the object plane uniformly. Alternatively, the illumination pattern may comprise a harmonically modulated intensity aligned with one or more selected directions. In some instances, the direction may be selected to improve resolution along a particular direction in the object plane (e.g., directions connecting the nearest neighbors in an array-shaped object).

In some instances, the imaging system may be configured to generate a harmonically modulated illumination intensity pattern (e.g., a sinusoidal-modulated illumination intensity pattern generated by a first optical transformation device (or illumination mask)) and may be used to image an object at enhanced resolution in a single scan without the need of computationally reconstructing the enhanced resolution image from a plurality of images. In some instances, the imaging system may comprise a second optical transformation device (e.g., a harmonically modulated phase mask or a harmonically modulated amplitude mask (or detection mask)) with a spatial frequency and orientation matching that of the harmonically modulated intensity in each region of the illumination pattern. In some instances, a detection mask may comprise a mask that is complementary to the illumination mask (i.e., a mask that is phase-shifted by 90 degrees relative to the illumination mask). In some instances, when scanning the object and acquiring a series of “instantaneous” images with the harmonically modulated intensity illumination pattern (i.e., the plurality of optical images presented to the sensor during the course of a single scan; at each point during the scan, the object is in a different position relative to the illumination pattern and the second optical transformation device, so these “instantaneous” images are not identical and are not simply shifted versions of the same image) the enhanced resolution image is generated by analog phase demodulation of the series of “instantaneous” images without the need of computationally-intensive resources. In some instances, the enhanced-resolution image may be reconstructed from the analog phase demodulation using a Fourier re-weighting technique that is computationally inexpensive.

In some examples, the imaging system may comprise methods of processing images captured by one or more image sensors. In some instances, the location of a photon reflected, transmitted, scattered, or emitted by the object may not accurately map to the corresponding location on the one or more image sensors. In some cases, photons may be re-mapped or reassigned to precisely determine the location of a photon reflected from the object. In some instances, a maximum-likelihood position of a fluorescent molecule (or other source of optical signal) can be, for example, midway between the laser beam center point in the object plane and the corresponding photon detection center point in the image plane. Photon reassignment in confocal imaging is described in, for example, Sheppard, et al., Super-resolution in Confocal Imaging, International Journal for Light and Electron Optics (1988); Sheppard, et al., Super resolution by image scanning microscopy using pixel reassignment, Optics Letters (2013); and Azuma and Kei, Super-resolution spinning-disk confocal microscopy using optical photon reassignment, Optics Express 23(11):15003-15011; each of which is incorporated herein by reference in its entirety.

### Exemplary Optical Transform Imaging System Configuration

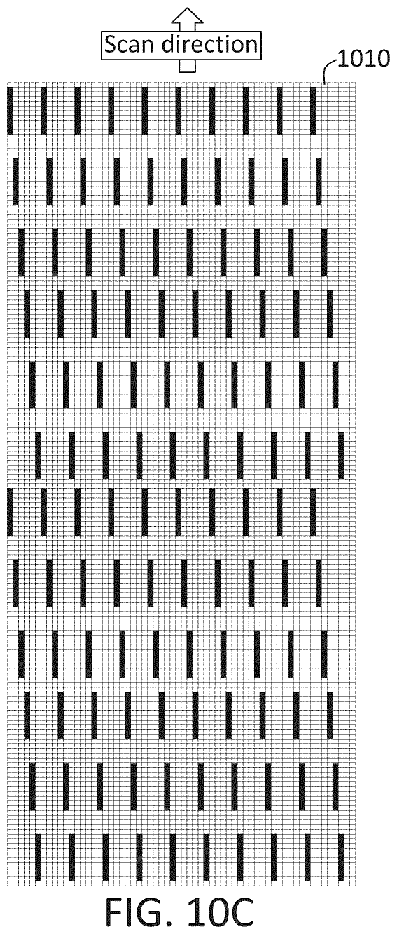

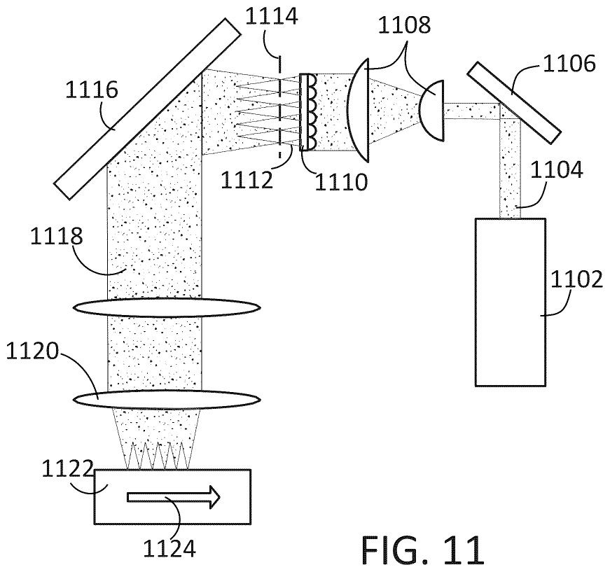

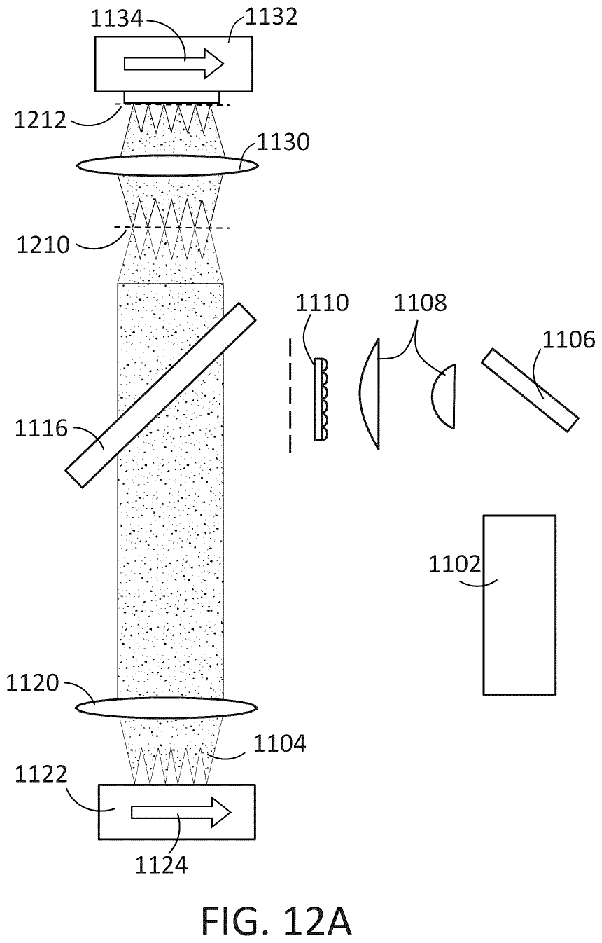

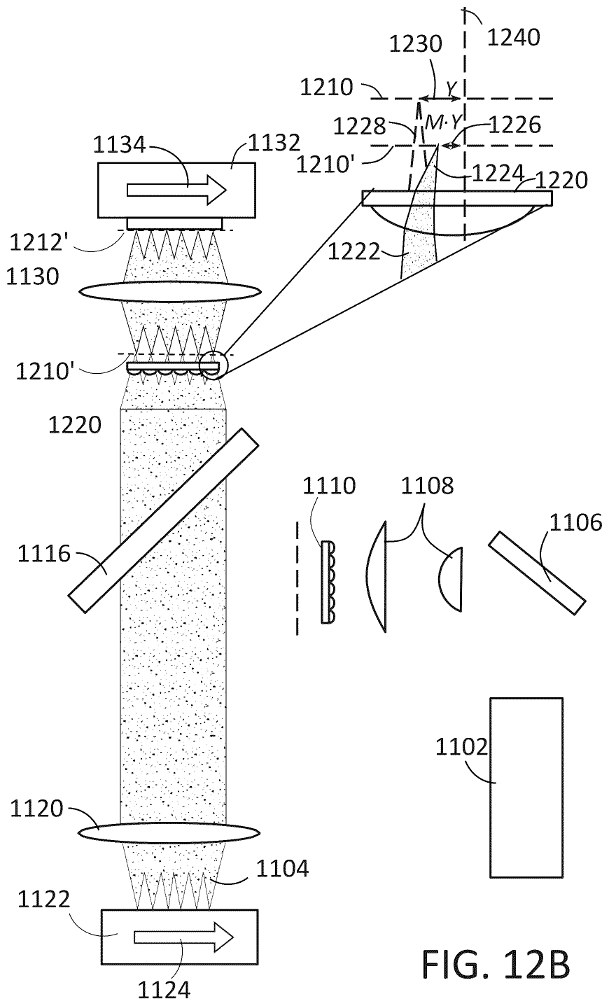

FIG. 11 provides a non-limiting schematic illustration of the excitation optical path for an optical transform imaging system with a radiation source configured in a reflection geometry, in accordance with some implementations of the disclosed imaging systems. In FIG. 11, the optical path of illumination light 1104 provided by radiation source 1102 is shown. Starting from the radiation source 1102 (e.g., a laser), illumination light 1104 is reflected from mirror 1106 through optical components 1108 (e.g., two plano-convex lenses configured as a beam expander) to a first optical transformation device 1110 (e.g., a micro-lens array) to produce patterned illumination 1112 at an intermediate focal plane 1114. The patterned illumination is then reflected from dichroic mirror 1116 as patterned light beam 1118 and focused by an objective 1120 (e.g., a compound objective) onto the object 1122. Object 1122 is translated relative to the optical assembly in direction 1124, which is aligned with the direction of photoelectron charge transfer 1134 in TDI image sensor 1132 to prevent blurring of the object in the image. In some instances, the optical assembly may be translated relative to the object to generate the relative motion in direction 1124. Light that is emitted by object 1122 in response to illumination by the patterned light beam 1118 is collected by objective 1120 and passed through dichroic mirror 1116 to tube lens 1130, which images the light onto TDI image sensor 1132, as will be described in more detail with respect to FIG. 12A.

FIG. 12A provides a schematic illustration of the emission optical pathway for an optical transform imaging system with a radiation source 1102 configured in a reflection geometry, and comprising only one micro-lens array (e.g., 1110) and additional optical components (e.g., mirror 1106 and lenses 1108) in the illumination pathway that produces an illumination light pattern 1112 (shown in FIG. 11) to illuminate object 1122. Patterned light 1104 is emitted by object 1122 in response to being illuminated and is collected by objective 1120, transmitted through dichroic mirror 1116, and focused on image plane 1210 (which may be a virtual image plane). The photons incident on image plane 1210 are relayed by tube lens 1130 and focused onto image plane 1212, which coincides with the positioning of TDI image sensor 1132. Object 1122 is translated relative to the optical assembly in direction 1124, which is aligned with the direction of photoelectron charge transfer 1134 in TDI image sensor 1132 to prevent blurring of the object in the image. In some instances, the optical assembly may be translated relative to the object to generate the relative motion in direction 1124.

In contrast to FIG. 12A, FIG. 12B provides a schematic illustration of the emission optical pathway for an optical transform imaging system which comprises two micro-lens arrays (e.g., a first micro-lens array 1110 in the illumination pathway, and a second micro-lens array 1220 in the emission pathway). In some instances, the first micro-lens array 1110 may alternatively be a diffraction grating. Again, as in FIG. 12A, the imaging system comprises a radiation source 1102 configured in a reflection geometry, and further comprises a first micro-lens array (e.g., 1110) and additional optical components (e.g., mirror 1106 and lenses 1108) in the illumination pathway that produce an illumination light pattern 1112 (shown in FIG. 11) to illuminate object 1122. Patterned light 1104 (e.g., a plurality of signal intensity maxima) is emitted by all or a portion of object 1122 in response to being illuminated and is collected by objective 1120, transmitted through dichroic mirror 1116 and a second micro-lens array 1220, and focused on image plane 1210′ (which may be a virtual image plane). The photons incident on image plane 1210′ are relayed by tube lens 1130 and focused onto image plane 1212′, which coincides with the positioning of TDI image sensor 1132. Object 1122 is translated relative to the optical assembly in direction 1124, which is aligned with the direction of photoelectron charge transfer 1134 in TDI image sensor 1132 to prevent blurring of the object in the image. In some instances, the optical assembly may be translated relative to the object to generate the relative motion in direction 1124.

The inset in FIG. 12B provides an exploded view of a portion of the emission optical path comprising a single micro-lens of micro-lens array 1220. The incident light 1222 is refocused (e.g., redirected) 1224 by the single micro-lens onto image plane 1210′ at a point 1226 that is spatially offset from the micro-lens optical axis 1240 by a distance of M·Y (where M is the demagnification factor) compared to the point 1230 on image plane 1210 (at a distance of Y from the micro-lens optical axis 1240) where the light 1228 would have been focused in the absence of the second micro-lens array 1220 (e.g., micro-lens array 1220 will reroute and redistribute light received from the object). The second optical transformation device (i.e., the second micro-lens array 1220 in this non-limiting example) thus compensates for a spatial offset (and corresponding loss of image resolution) that would have been observed for individual pixels in the TDI image sensor in an otherwise identical imaging system that lacked the second optical transformation device. In some instances, the second optical transformation device (i.e., the second micro-lens array 1220) may compensate for a spatial offset (and corresponding loss of image resolution) that would have been observed for the plurality of signal intensity maxima output from the at least a portion of the object 1122 in response to being illuminated.

### Patterned Illumination Optical Assembly

As described with respect to the exemplary imaging system illustrated in FIG. 1 (and other imaging system configurations described herein, see, e.g., FIGS. 2, 3A, 3B, 4, 5, 11, 12A, and 12B), the illumination unit 102 (or patterned illumination optical assembly) may comprise a light source (or radiation source) 104 and a first optical transformation device 106, as well as addition optics 108, or any combination thereof.

In some instances, the illumination unit may comprise one or more light sources or radiation sources, e.g., 1, 2, 3, 4, or more than 4 light sources or radiation sources. In some instances, the one or more light source or radiation source may be a laser, a set of lasers, an incoherent source, or any combination thereof. In some instances, the incoherent source may be a plasma-based light source. In some instances, the one or more light sources or radiation sources may provide radiation at one or more specific wavelengths for absorption by exogenous contrast fluorescence dyes. In addition, the one or more light sources or radiation sources may provide radiation at a particular wavelength for endogenous fluorescence, auto-fluorescence, phosphorous, or any combination thereof. In some instances, the one or more light sources or radiation sources may provide continuous wave, pulsed, Q-switched, chirped, frequency-modulated, amplitude-modulated, harmonic, or any combination thereof of output light or radiation.

In any of the imaging system configurations described herein, the one or more light sources (or radiation sources, etc.) may produce light at a center wavelength ranging from about 400 nanometers (nm) to about 1,500 nm or any range thereof. In some instances, the center wavelength may be about 400 nm, 500 nm, 600 nm, 700 nm, 800 nm, 900 nm, 1,000 nm, 1,100 nm, 1,200 nm, 1,300 nm, 1,400 nm, or 1,500 nm. Those of skill in the art will recognize that the center wavelength may be any value within this range, e.g., about 633 nm.

In any of the imaging system configurations described herein, the one or more light sources (or radiation sources), alone or in combination with one or more optical components (e.g., optical filters and/or dichroic beam splitters), may produce light at the specified center wavelength within a bandwidth of ±2 nm, ±5 nm, ±10 nm, ±20 nm, ±40 nm, ±80 nm, or greater. Those of skill in the art will recognize that the bandwidth may have any value within this range, e.g., ±18 nm.

In any of the imaging system configurations described herein, the first and/or second optical transformation device may comprise one or more of a micro-lens array (MLA), diffractive element (e.g., a diffraction grating), digital micromirror device (DMD), phase mask, amplitude mask, spatial light modulator (SLM), pinhole array, or any combination thereof.

In some instances, the first and/or second optical transformation device in any of the imaging system configurations described herein may comprise a micro-lens array (MLA). In some instances, an MLA optical transformation device may comprise a plurality of micro-lenses 700 or 703 configured in a plurality of rows and columns, as seen for example in FIGS. 7A-7B.

In some instances, the MLA may comprise about 200 columns to about 4,000 columns of micro-lenses or any range thereof. In some instances, the MLA may comprise at least about 200 columns, 400 columns, 600 columns, 800 columns, 1,000 columns, 1,200 columns, 1,500 columns, 1,750 columns, 2,000 columns, 2,200 columns, 2,400 columns, 2,600 columns, 2,800 columns, 3,000 columns, 3,250 columns, 3,500 columns, 3,750 columns, or 4,000 columns of micro-lenses. In some instances, the MLA may comprise at most about 200 columns, 400 columns, 600 columns, 800 columns, 1,000 columns, 1,200 columns, 1,500 columns, 1,750 columns, 2,000 columns, 2,200 columns, 2,400 columns, 2,600 columns, 2,800 columns, 3,000 columns, 3,250 columns, 3,500 columns, 3,750 columns, or 4,000 columns of micro-lenses. Those of skill in the art will recognize that the MLA may comprise any number of columns within this range, e.g., about 2,600 columns. In some instances, the number of columns in the MLA may be determined by the size of the pupil plane (e.g., the number and organization of pixels in the pupil plane).

In some instances, the MLA may comprise about 2 rows to about 50 rows of micro-lenses, or any range thereof. In some instances, the MLA may comprise at least about 2 rows, 4 rows, 6 rows, 8 rows, 10 rows, 12 rows, 14 rows, 16 rows, 18 rows, 20 rows, 22 rows, 24 rows, 26 rows, 28 rows, 30 rows, 32 rows, 34 rows, 36 rows, 38 rows, 40 rows, 42 rows, 44 rows, 46 rows, 48 rows, or 50 rows of micro-lenses. In some instances, the MLA may comprise at most about 2 rows, 4 rows, 6 rows, 8 rows, 10 rows, 12 rows, 14 rows, 16 rows, 18 rows, 20 rows, 22 rows, 24 rows, 26 rows, 28 rows, 30 rows, 32 rows, 34 rows, 36 rows, 38 rows, 40 rows, 42 rows, 44 rows, 46 rows, 48 rows, or 50 rows of micro-lenses. Those of skill in the art will recognize that the MLA may comprise any number of rows within this range, e.g., about 32 rows. In some instances, the abovementioned values, and ranges thereof, for the rows and columns of micro-lenses may be reversed.

In some instances, the MLA may comprise a pattern of micro-lenses (e.g., a staggered rectangular or a tilted hexagonal pattern) that may comprise a length of about 4 mm to about 100 mm, or any range thereof. In some instances, the pattern of micro-lenses in an MLA may comprise a length of at least about 4 mm, 8 mm, 12 mm, 16 mm, 20 mm, 30 mm, 40 mm, 50 mm, 60 mm, 70 mm, 80 mm, 90 mm, or 100 mm. In some instances, the pattern of micro-lenses in an MLA may comprise a length of at most about 4 mm, 8 mm, 12 mm, 16 mm, 20 mm, 30 mm, 40 mm, 50 mm, 60 mm, 70 mm, 80 mm, 90 mm, or 100 mm. Those of skill in the art will recognize that the pattern of micro-lenses in the MLA may have a length of any value within this range, e.g., about 78 mm. In some instances, the length of the pattern of micro-lenses in the MLA may be determined with respect to a desired magnification. For example, the length of the pattern of micro-lenses in the MLA may be 2.6 mm×magnification.

In some cases, the pattern (e.g., the staggered rectangular or the tilted hexagonal pattern) of micro-lenses in an MLA may comprise a width of about 100 μm to about 1500 μm or any range thereof. In some instances, the pattern of micro-lenses in an MLA may comprise a width of at most about 100 μm, 150 μm, 200 μm, about 250 μm, 300 μm, about 350 μm, about 400 μm, 450 μm, or 500 μm. In some instances, the pattern (e.g., staggered rectangular or tilted hexagonal pattern) of micro-lenses in an MLA may comprise a width of at least about 100 μm, 150 μm, 200 μm, 250 μm, 300 μm, 350 μm, 400 μm, 450 μm, or 500 μm. Those of skill in the art will recognize that the pattern of micro-lenses in the MLA may have a width of any value within this range, e.g., about 224 μm. In some instances, the width of the MLA pattern may be determined with respect to a desired magnification, e.g., 50 μm×magnification (i.e., similar to the determination of the length of the pattern of micro-lenses in the MLA).

In some examples, the tilted hexagonal pattern of micro-lenses in an MLA may be tilted at an angle 702 with respect to the vertical axis of the MLA. For example, the angle (θ) of the tilted hexagonal patterned MLA may be determined by the following:

$$θ = {tan}^{- 1} ( \frac{1}{\sqrt{3} ( 2 N + 1 )} )$$

where N is a number of rows of micro-lenses in the tilted hexagonal pattern as described above.

In some instances, the angle (θ) of the tilted hexagonal pattern MLA may be configured to be about 0.5 degrees to about 45 degrees or any range thereof. In some instances, the angle (θ) of the tilted hexagonal pattern MLA may be configured to be at most about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 20, 25, 30, 35, 40, or 45 degrees. In some instances, the angle (θ) of the tilted hexagonal pattern MLA may be configured to be at least about 0.5, 1 degree, 1.5, 2, about 2.5, about 3, about 3.5, about 4, about 4.5, about 5, 5.5, 6, 6.5, 7, 7.5, 8, 8.5, 9, 9.5, 10, 10.5, 11, 11.5, 12, 12.5, 13, 13.5, 14, 14.5, 15, 20, 25, 30, 35, 40, or 45 degrees. Those of skill in the art will recognize that the angle (θ) of the tilted hexagonal pattern MLA may have any value within this range, e.g., about 4.2 degrees. In some instances, the angle (θ) of the tilted hexagonal pattern may be configured to generate an illumination pattern with even spacing between illumination peaks in a cross-scan direction.

In some instances, the MLA may be further characterized by pitch, micro-lens diameter, numerical aperture (NA), focal length, or any combination thereof. In some instances, a micro-lens of the plurality of micro-lenses may have a diameter of about 5 micrometers (μm) to about 40 μm, or any range thereof. In some instances, a micro-lens of the plurality of micro-lenses may have a diameter of at most about 5 μm, 10 μm, 15 μm, 20 μm, 25 μm, 30 μm, 35 μm, or 40 μm. In some instances, a micro-lens of the plurality of micro-lenses may have a diameter of at least about 5 μm, 10 μm, 15 μm, 20 μm, 25 μm, 30 μm, 35 μm, or 40 μm. Those of skill in the art will recognize that the diameters of micro-lenses may have any value within this range, e.g., about 28 μm. In some instances, each micro-lens in a plurality of micro-lenses in an MLA has a same diameter. In some instances, at least one micro-lens in a plurality of micro-lenses in an MLA has a different diameter from another micro-lens in the plurality.

In some instances, the distances between adjacent micro-lenses may be referred to as the pitch of the MLA. In some instances, the pitch of the MLA may be about 10 μm to about 70 μm or any range thereof. In some instances, the pitch of the MLA may be at least about 10 μm, 15 μm, 20 μm, 25 μm, 30 μm, 35 μm, 40 μm, 45 μm, 50 μm, 55 μm, 60 μm, 65 μm, or 70 μm. In some instances, the pitch of the MLA may be at most about 10 μm, 15 μm, 20 μm, 25 μm, 30 μm, 35 μm, 40 μm, 45 μm, 50 μm, 55 μm, 60 μm, 65 μm, or 70 μm. Those of skill in the art will recognize that the distances between adjacent micro-lenses in the MLA may have any value within this range, e.g., about 17 μm.

In some instances, the pitch(or spacing) of the individual lenses in the one or more micro-lens arrays of the disclosed systems may be varied to change the distance between illumination peak intensity locations and in addition may adjust (e.g., increase) the lateral resolution of the imaging system. In some instances, for example, the lateral resolution of the imaging system may be improved by increasing the pitch between individual lenses of the one or more micro-lens arrays.

In some instances, the numerical aperture (NA) of micro-lenses in the MLA may be about 0.01 to about 2.0 or any range thereof. In some instances, the numerical aperture of the micro-lenses in the MLA may be at least 0.01, 0.05, 0.1, 0.2, 0.3, 0.4, 0.5, 0.6, 0.7, 0.8, 0.9, 1.0, 1.5, 1.6, 1.7, 1.8, 1.9, or 2.0. In some instances, the numerical aperture of the micro-lenses in the MLA may be at most 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05, or 0.01. In some instances, the NA of micro-lenses in the MLA may be about 0.04, 0.05, 0.06, 0.07, 0.08, 0.09, 0.1, 0.11, or 0.12. Those of skill in the art will recognize that the NA of the micro-lenses in the MLA may have any value within this range, e.g., about 0.065.

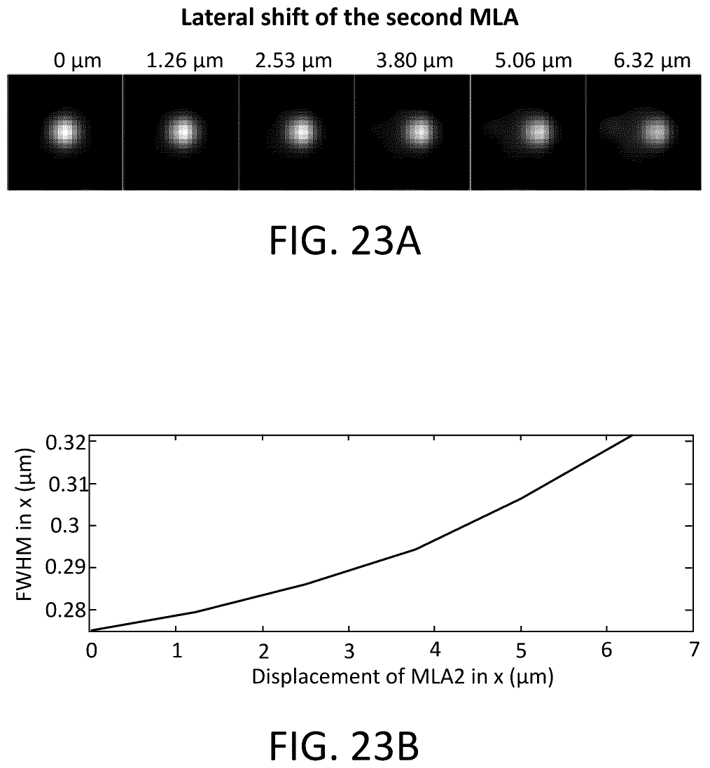

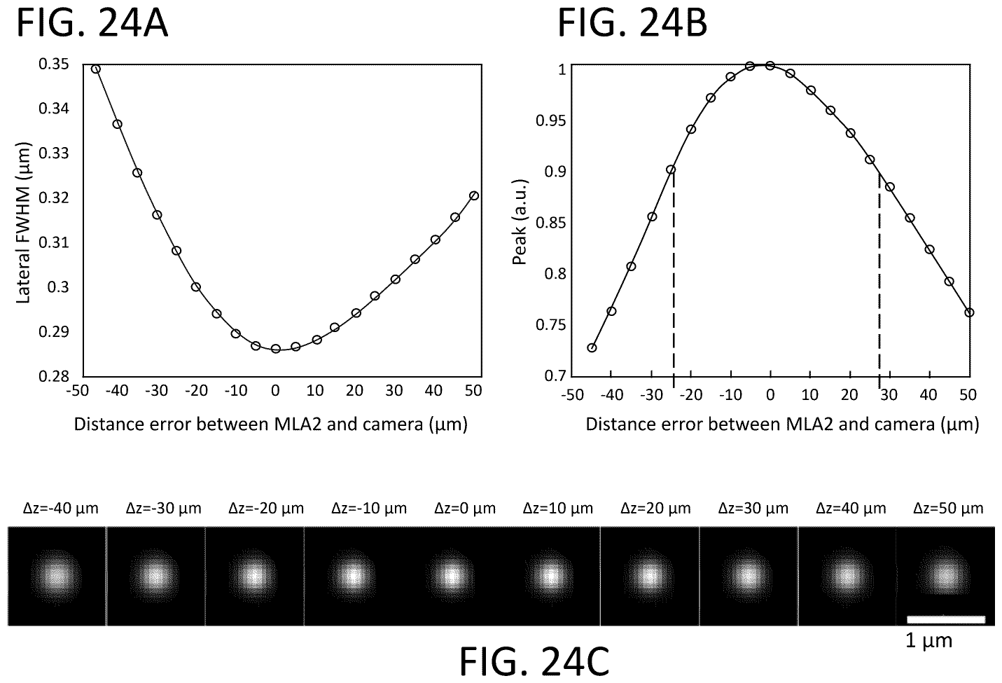

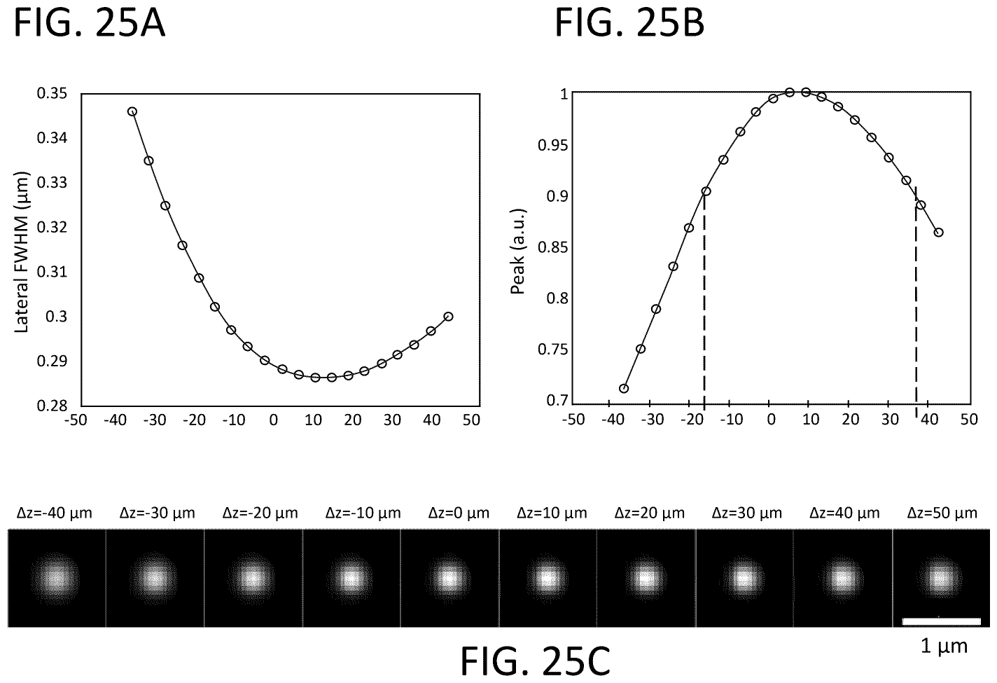

In some instances, specifying tighter manufacturing tolerances for micro-lens array specifications (e.g., tighter manufacturing tolerances on individual microlens shape, pitch, numerical aperture, and/or the spacing between rows and columns of micro-lenses in the array) may provide for improved imaging performance, e.g., by eliminating artifacts such as star patterns or other non-symmetrical features in the illumination PSF that contribute to cross-talk between adjacent objects (such as adjacent sequencing beads). In some instances, the tolerable variation in MLA pitch is ±20% and the tolerable variation in focal length is ±15% (see e.g., Example 3 with regards to FIGS. 23A and 23B and to FIGS. 24A-25C, respectively).

In some instances, the use of a pinhole aperture array positioned on or in front of the image sensor, e.g., a pinhole aperture array that mirrors the array of micro-lenses in a microlens array (MLA) positioned in the optical path upstream from the image sensor, may be used to minimize or eliminate artifacts in the system PSF (see Example 3). In some instances, the pinhole aperture array may comprise a number of apertures that are equal to the number of micro-lenses in the MLA. In some instances, the apertures in the pinhole aperture array may be positioned in the same pattern and at the same pitch used for the micro-lenses in the MLA.

In some instances, the pinhole apertures in the aperture array may have diameters ranging from about 0.1 μm to about 2.0 μm. In some instances, the pinhole apertures in the aperture array may have diameters of at least 0.1 μm, 0.15 μm, 0.2 μm, 0.25 μm, 0.3 μm, 0.35 μm, 0.4 μm, 0.45 μm, 0.5 μm, 0.55 μm, 0.6 μm, 0.65 μm, 0.7 μm, 0.75 μm, 0.8 μm, 0.85 μm, 0.9 μm, 0.95 μm, 1.0 μm, 1.05 μm, 1.1 μm, 1.15 μm, 1.2 μm, 1.25 μm, 1.3 μm, 1.35 μm, 1.4 μm, 1.45 μm, 1.5 μm, 1.55 μm, 1.6 μm, 1.65 μm, 1.7 μm, 1.75 μm, 1.8 μm, 1.85 μm, 1.9 μm, 1.95 μm, or 2.0 μm. In some instances, the pinhole apertures in the aperture array may have diameters of at most 2.0 μm, 1.95 μm, 1.9 μm, 1.85 μm, 1.8 μm, 1.75 μm, 1.7 μm, 1.65 μm, 1.6 μm, 1.55 μm, 1.5 μm, 1.45 μm, 1.4 μm, 1.35 μm, 1.3 μm, 1.25 μm, 1.2 μm, 1.15 μm, 1.1 μm, 1.05 μm, 1.0 μm, 0.95 μm, 0.9 μm, 0.85 μm, 0.8 μm, 0.75 μm, 0.7 μm, 0.65 μm, 0.6 μm, 0.55 μm, 0.5, μm 0.45 μm, 0.4 μm, 0.35 μm, 0.3 μm, 0.25 μm, 0.2 μm, 0.15 μm, 0.1 μm. Those of skill in the art will recognize that the pinhole apertures in the aperture array may have diameters of any value within this range, e.g., about 1.26 μm.

### Projection Optical Assembly

As described with respect to the exemplary imaging system illustrated in FIG. 1 (and other imaging system configurations described herein, see, e.g., FIGS. 2, 3A, 3B, 4, 5, 11, 12A, and 12B), the projection unit 120 (or projection optical assembly) is configured to direct the patterned illumination to the object being imaged, and to receive the reflected, transmitted, scattered, or emitted light to be directed to the detector. In some instances, the projection optical assembly may comprise a dichroic mirror, an object-facing optical element, and one or more relay optical components, or any combination thereof.

In some examples, the projection optical assembly may comprise a dichroic mirror configured to transmit patterned light in one wavelength range and reflect patterned light in another wavelength range. In some instances, the dichroic mirror may comprise one or more optical coatings that may reflect or transmit a particular bandwidth of radiative energy. Non-limiting examples of paired transmittance and reflectance ranges for the dichroic mirror include 425-515 nm and 325-395 nm, 454-495 nm and 375-420 nm, 492-510 nm and 420-425 nm, 487-545 nm and 420-475 nm, 520-570 nm and 400-445 nm, 512-570 nm and 440-492 nm, 512-570 nm and 455-500 nm, 520-565 nm and 460-510 nm, 531-750 nm and 480-511 nm, 530-595 nm and 470-523 nm, 537-610 nm and 470-523 nm, 550-615 nm and 480-532 nm, 567-620 nm and 490-550 nm, 575-650 nm and 500-560 nm, 587-650 nm and 500-565 nm, 587-650 nm and 500-565 nm, 592-660 nm and 540-582 nm, 592-660 nm and 540-582 nm, 612-675 nm and 532-585 nm, 608-700 nm and 525-588 nm, 619-680 nm and 540-590 nm, 647-710 nm and 575-626 nm, 620-720 nm, and 570-646 nm, 667-725 nm and 585-650 nm, 673-740 nm and 600-652 nm, 686-745 nm and 615-669 nm, 692-760 nm and 590-667 nm, 705-780 nm and 630-684 nm, and 765-860 nm and 675-737 nm.

In some instances, the dichroic mirror may have a length of about 10 mm to about 250 mm or any range thereof. In some instances, the dichroic mirror may have a length of at least about 10 mm, 20 mm, 30 mm, 40 mm, 50 mm, 60 mm, 70 mm, 80 mm, 100 mm, 150 mm, 200 mm, or 250 mm. In some instances, the dichroic mirror may have a length of at most about 10 mm, 20 mm, 30 mm, 40 mm, 50 mm, 60 mm, 70 mm, 80 mm, 100 mm, 150 mm, 200 mm, or 250 mm. Those of skill in the art will recognize that the dichroic mirror may be any length within this range, e.g., 54 mm.

In some instances, the dichroic mirror may have a width of about 10 mm to about 250 mm or a range thereof. In some instances, the dichroic mirror may have a width of at least about 10 mm, 20 mm, 30 mm, 40 mm, 50 mm, 60 mm, 70 mm, 80 mm, 100 mm, 150 mm, 200 mm, or 250 mm. In some instances, the dichroic mirror may have a width of at most about 10 mm, 20 mm, about 30 mm, about 40 mm, about 50 mm, about 60 mm, about 70 mm, about 80 mm, about 100 mm, about 150 mm, about 200 mm, or about 250 mm. Those of skill in the art will recognize that the dichroic mirror may be any width within this range, e.g., 22 mm.

In some instances, the dichroic mirror may be comprised of fused silica, borosilicate glass, or any combination thereof. The dichroic mirror may be tailored to a particular type of fluorophore or dye being used in an experiment. The dichroic mirror may be replaced by one or more optical elements (e.g., optical beam splitter or coating, wave plate, etc.) capable of and configured to direct an illumination pattern from the pattern illumination source to the object and direct the reflected pattern from the object to the detection unit.

In some instances, the projection optical assembly may comprise an object-facing optical component configured to direct the illumination pattern to, and receive the light reflected by, transmitted by, scattered from, or emitted from, the object. In some instances, the object-facing optics may comprise an objective lens or a lens array. In some instances, the objective lens may have a numerical aperture of about 0.2 to about 2.4. In some instances, the objective lens may have a numerical aperture of at least about 0.2, 0.4, 0.6, 0.8, 1, 1.2, 1.4, 1.6, 1.8, 2, 2.2, or 2.4. In some instances, the objective lens may have a numerical aperture of at most about 0.2, 0.4, 0.6, 0.8, 1, 1.2, 1.4, 1.6, 1.8, 2, 2.2, or 2.4. Those of skill in the art will recognize that the objective lens may have any numerical aperture within this range, e.g., 1.33.

In some instances, the objective lens aperture may be filled by an illumination pattern covering the total usable area of the objective lens aperture while maintaining well separated intensity peaks of the illumination pattern. In some instances, the tube lens or relay optics of the projection optical assembly may be configured to relay the patterned illumination to the objective lens aperture to fill the total usable area of the objective lens aperture while maintaining well separated illumination intensity peaks.

### Detection Unit

As described with respect to the exemplary imaging system illustrated in FIG. 1 (and other imaging system configurations described herein, see, e.g., FIGS. 2, 3A, 3B, 4, 5, 11, 12A, and 12B), the detection unit 140 (or patterned illumination detector) may comprise a second optical transformation device 142, one or more image sensors 144 configured for performing TDI imaging, optional optics 148, or any combination thereof.

In any of the imaging system configurations described herein, the detection unit may comprise one or more image sensors 144 as illustrated in FIG. 1. In some instances, the one or more image sensors may comprise a time delay and integration (TDI) camera, charge-coupled device (CCD) camera, complementary metal-oxide semiconductor (CMOS) camera, a single-photon avalanche diode (SPAD) array, or any combination thereof. In some instances, the detection unit may comprise one or more image sensors configured to detect photons in the visible, near-infrared, infrared or any combination thereof. In some instances, each of two or more image sensors may be configured to detect photons in the same wavelength range. In some instances, each of two or more image sensors may be configured to detect photons in a different wavelength range.

In any of the imaging system configurations described herein, the one or more image sensors may each comprise from about 256 pixels to about 65,000 pixels. In some instances, an image sensor may comprise at least 256 pixels, 512 pixels, 1,024 pixels, 2,048 pixels, 4,096 pixels, 8,192 pixels, 16,384 pixels, 32,768 pixels, or 65,536 pixels. In some instances, an image sensor may comprise at most 256 pixels, 512 pixels, 1,024 pixels, 2,048 pixels, 4,096 pixels, 8,192 pixels, 16,384 pixels, 32,768 pixels, or 65,536 pixels. Those of skill in the art will recognize that an image sensor may have any number of pixels within this range, e.g., 2,048 pixels.

In any of the imaging system configuration described herein, the one or more image sensors may have a pixel size of about 1 micrometer (μm) to about 7 μm. In some cases, the sensor may have a pixel size of at least about 1 μm, 2 μm, 3 μm, 4 μm, 5 μm, 6 μm, or 7 μm. In some instances, the sensor may have a pixel size of at most about 1 μm, 2 μm, 3 μm, 4 μm, 5 μm, 6 μm, or 7 μm. Those of skill in the art will recognize that the objective lens may have any pixel size within this range, e.g., about 1.4 μm.

In any of the imaging system configurations described herein, the one or more image sensors may operate on a TDI clock cycle (or integration time) ranging from about 1 nanosecond (ns) to about 1 millisecond (ms). In some instances, the TDI clock cycle may be at least Ins, 10 ns, 100 ns, 1 microsecond (μs), 10 μs, 100 μs, 1 ms, 10 ms, 100 ms, or 1 s. Those of skill in the art will recognize that the TDI clock cycle may have any value within this range, e.g., about 12 ms. In any of the imaging system configurations described herein, the one or more sensors may comprise TDI sensors that include a number of stages used to integrate charge during image acquisition. For example, in some instances, the one or more TDI sensors may comprise at least 64 stages, at least 128 stages, at least 256 stages. In some instances, the one or more TDI sensors may be split into two or more (e.g., 2, 3, 4, or more than 4) parallel sub-sensors that can be triggered sequentially to reduce motion-induced blurring of the image, where the time delay between sequential triggering is proportional to the relative rate of motion between the sample to be imaged and the one or more TDI sensors.

In any of the imaging system configurations described herein, the system may be configured to acquire one or more imaged with a scan time ranging from about 0.1 millisecond (ms) to about 100 sec. In some instances, the image acquisition time (or scan time) may be at least 0.1 ms, 1 ms, 10 ms, 100 ms, 1 microsecond (μs), 10 μs, 100 μs, 1 s, 10 s, or 100 s. In some instances, the image acquisition time (or scan time) may have any value within the range of values described in this paragraph, e.g., 2.4 s.

In any of the imaging system configurations described herein, the optional optics included in the detection unit may comprise a plurality of relay lenses, a plurality of tube lenses, a plurality of optical filters, or any combination thereof. In some cases, the sensor pixel size and magnification of the imaging system may be configured to allow for adequate sampling of optical light intensity at the sensor imaging plane. In some instances, the adequate sampling may be approaching or substantially exceeding the Nyquist sampling frequency.

In any of the imaging system configurations described herein, the second optical transformation device 142 may comprise one or more of a micro-lens array (MLA), diffractive element, digital micromirror device (DMD), phase mask, amplitude mask, spatial light modulator (SLM), pinhole array, or other transformation elements, etc. The second optical transformation device may transform an illumination pattern generated by a first optical transformation device 106, and patterned light reflected, transmitted, scattered, or emitted from the object to an array of intensity peaks that are non-overlapping. In some instances, the second optical transformation device 142 may comprise an optical transformation device that is complementary to the first optical transformation device 106 in the pattern illumination source 102. In some instances, the first and second optical transformation devices may be the same type of optical transformation device (e.g., micro-lens array). In some instances, the complementary first and second optical transformation devices may share common characteristics, such as the characteristics of the first optical transformation device 106 described elsewhere herein.

The first optical transformation device of the disclosed imaging systems may be configured to apply a first transformation to generate an illumination pattern that may be further transformed by the second optical transformation device. The first and second transformations by the first and second optical transformation devices may generate an enhanced resolution image of the object, compared to an image of the object generated without the use of these optical transformation devices. The resolution enhancement resulting from the inclusion of these optical transformation devices are seen in a comparison of FIGS. 9A and 9B, which shows an image of an object generated using two optical transformation devices (FIG. 9B) and an image of an object generated using a first optical transformation device only (FIG. 9A).

In any of the imaging system configuration described herein, the detection unit 140 as illustrated in FIG. 1 may be configured so that the one or more image sensors 144 detect light at one or more center wavelengths ranging from about 400 nanometers (nm) to about 1,500 nm or any range thereof. In some instances, the center wavelength may be at least about 400 nm, 500 nm, 600 nm, 700 nm, 800 nm, 900 nm, 1,000 nm, 1,100 nm, 1,200 nm, 1,300 nm, 1,400 nm, or 1,500 nm. In some instances, the center wavelength may be at most about 400 nm, 500 nm, 600 nm, 700 nm, 800 nm, 900 nm, 1,000 nm, 1,100 nm, 1,200 nm, 1,300 nm, 1,400 nm, or 1,500 nm. Those of skill in the art will recognize that the objective lens may have any pixel size within this range, e.g., about 703 nm.

In any of the imaging system configurations described herein, the one or more image sensors, alone or in combination with one or more optical components (e.g., optical filters and/or dichroic beam splitters), may detect light at the specified center wavelength(s) within a bandwidth of ±2 nm, ±5 nm, ±10 nm, ±20 nm, ±40 nm, ±80 nm, or greater. Those of skill in the art will recognize that the bandwidth may have any value within this range, e.g., ±18 nm.

In any of the imaging system configurations described herein, the amount of light reflected, transmitted, scattered, or emitted by the object that reaches the one or more image sensors is at least 40%, 50%, 60%, 70%, 80%, or 90% of the reflected, transmitted, scattered, or emitted light entering the detection unit.

In any of the imaging system configurations disclosed herein, the imaging throughput (in terms of the number of distinguishable features or locations that can be imaged (or “read”) per second) may range from about 106 reads/s to about 1010 reads/s. In some instances, the imaging throughput may be at least about 106, at least 5×10⁶, at least 10⁷, at least 5×10⁷, at least 10⁸, at least 5×10⁸, at least 10⁹, at least 5×10⁹, or at least 10¹⁰ reads/s. Those of skill in the art will recognize that the imaging throughput may be of any value within this range, e.g., about 2.13×10⁹ reads/s.

In any of the imaging system configurations described herein, the imaging system may be capable of integrating signal and acquiring scanned images having an increased signal-to-noise ratio (SNR) compared to a signal-to-noise ratio (SNR) in images acquired by an otherwise identical imaging system that lacks the second optical transformation device. In some instances, the signal-to-noise ratio (SNR) exhibited by the scanned images acquired using the disclosed imaging systems is increased by greater than 20%, 40%, 60%, 80%, 100%, 120%, 140%, 160%, 180%, 200%, 300%, 400%, 500%, 600%, 700%, 800%, 900%, 1,000%, 1,200%, 1,400%, 1,600%, 1,800%, 2,000%, or 2500% relative to that of a scanned image acquired using an otherwise identical imaging system that lacks the second optical transformation device. In some instances, the signal-to-noise ratio (SNR) exhibited by the scanned images acquired using the disclosed imaging systems is increased by at least 2×, 3×, 4×, 5×, 6×, 7×, 8×, 9×, or 10× relative to that of a scanned image acquired using an otherwise identical imaging system that lacks the second optical transformation device.

In any of the imaging system configurations described herein, the imaging system may be capable of integrating signal and acquiring scanned images having an increased image resolution compared to the image resolution in images acquired by an otherwise identical imaging system that lacks the second optical transformation device. In some instances, the image resolution exhibited by the scanned images acquired using the disclosed imaging systems is increased by about 10%, 20%, 30%, 40%, 50%, 60%, 70%, 80%, 90%, 100%, 125%, 150%, 1275%, 200%, 225%, 250%, 275%, 300%, or more than 300% relative to that of a scanned image acquired using an otherwise identical imaging system that lacks the second optical transformation device. In some instances, the image resolution exhibited by the scanned images acquired using the disclosed imaging systems is increased by at least 1.2x, at least 1.5x, at least 2x, or at least 3x relative to that of a scanned image acquired using an otherwise identical imaging system that lacks the second optical transformation device.

In some instances, the image resolution exhibited by the scanned images acquired using the disclosed imaging systems is better than 0.6 (FWHM of the effective point spread function in units of A/NA), better than 0.5, better than 0.45, better than 0.4, better than 0.39, better than 0.38, better than 0.37, better than 0.36, better than 0.35, better than 0.34, better than 0.33, better than 0.32, better than 0.31, better than 0.30, better than 0.29, better than 0.28, better than 0.27, better than 0.26, better than 0.25, better than 0.24, better than 0.23, better than 0.22, better than 0.21, or better than 0.20. Those of skill in the art will recognize that the image resolution exhibited by the scanned images acquired using the disclosed imaging systems may be any value within this range, e.g., about 0.42 (FWHM of the effective point spread function in units of N/NA).

### Object Positioning System

In any of the imaging system configurations described herein, the object positioning system 130 as illustrated in FIG. 1 may comprise one or more actuators, e.g., a linear translational stage, two-dimensional translational stage, three-dimensional translational stage, circular rotation stage, or any combination thereof, configured to support and move the object 132 relative to the projection unit 120 (or vice versa).

In some instances, the one or more actuators may be configured to move the object (or projection optical assembly) over a distance ranging from about 0.1 mm to about 250 mm or any range thereof. In some instances, the one or more actuators may be configured to move the object (or projection optical assembly) at least 0.1 mm, 0.5 mm, 1 mm, 2 mm, 4 mm, 6 mm, 8 mm, 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, or 250 mm. In some instances, the one or more actuators may be configured to move the object (or projection optical assembly) at most about 250 mm, 240 mm, 230 mm, 220 mm, 210 mm, 200 mm, 190 mm, 180 mm, 170 mm, 160 mm, 150 mm, 140 mm, 130 mm, 120 mm, 110 mm, 100 mm, 90 mm, 80 mm, 70 mm, 60 mm, 50 mm, 40 mm, 30 mm, 20 mm, 10 mm, 8 mm, 6 mm, 4 mm, 2 mm, 1 mm, 0.5 mm, or 0.1 mm. Those of skill in the art will recognize that the one or more actuators may be configured to move the object (or projection optical assembly) over a distance having any value within this range, e.g., about 127.5 mm.

In some instances, the one or more actuators may travel with a resolution of about 20 nm to about 500 nm, or any range thereof. In some instances, the actuator may travel with a resolution of at least about 20 nm, 40 nm, 60 nm, 80 nm, 100 nm, 150 nm, 200 nm, 250 nm, 300 nm, 350 nm, 400 nm, or 500 nm. In some instances, the actuator may travel with a resolution of at most about 20 nm, 40 nm, 60 nm, 80 nm, 100 nm, 150 nm, 200 nm, 250 nm, 300 nm, 350 nm, 400 nm, or 500 nm. Those of skill in the art will recognize that the actuator may travel with a resolution of any value within this range, e.g., about 110 nm.

In some instances, the one or more actuators may be configured to translate the object (or projection optical assembly) at a rate of about 1 mm/s to about 220 mm/s or any range thereof. In some instances, the one or more actuators may be configured to translate the object (or projection optical assembly) at a rate of at least about 1 mm/s, 20 mm/s, 40 mm/s, 60 mm/s, 80 mm/s, 100 mm/s, 120 mm/s, 140 mm/s, 160 mm/s, about 180 mm/s, about 200 mm/s, or about 220 mm/s. In some instances, the one or more actuators may be configured to translate the object (or projection optical assembly) at a rate of at most about 1 mm/s, 20 mm/s, 40 mm/s, 60 mm/s, 80 mm/s, 100 mm/s, 120 mm/s, 140 mm/s, 160 mm/s, 180 mm/s, 200 mm/s, or 220 mm/s. Those of skill in the art will recognize that the one or more actuators may be configured to translate the object (or projection optical assembly) at a rate of any value within this range, e.g., about 119 mm/s.

### Methods of Imaging

Disclosed herein, in some examples, are methods of imaging an object with the imaging systems described herein. In some instances, imaging an object with the imaging systems described herein may provide high-throughput, high SNR imaging while maintaining an enhanced imaging resolution. In some cases, the method of imaging an object may comprise: (a) illuminating a first optical transformation device by a radiation source; (b) transforming light from the radiation source to generate an illumination pattern; (c) projecting the illumination pattern to a projection optical assembly configured to receive and direct the illumination pattern from the first optical transformation device to the object; (d) receiving a reflection of the illumination pattern from the object by a second optical transformation device; (c) transforming the illumination pattern by the second optical transformation device to generate a transformed illumination pattern; (f) detecting the transformed illumination pattern with one or more image sensors, wherein the image sensors are configured for time delay and integration (TDI) imaging, and wherein the illumination pattern is moved relative to the object and/or the object is moved relative to the illumination pattern. The illumination pattern and/or the object may be moved via one or more actuators. For example, the actuator may be a linear stage with the object attached thereto. Alternatively, the actuator may be rotational.

In some instances, imaging an object using the disclosed imaging systems may comprise: illuminating a first optical transformation device with a light beam, applying by the first optical transformation device a first optical transformation to the light beam to produce an illumination pattern, providing the illumination pattern to the object by an object-facing optical component onto the object, directing light reflected, transmitted, scattered, or emitted by (e.g., output from) the object to a second optical transformation device, applying by the second optical transformation device a second optical transformation to light reflected, transmitted, scattered, or emitted by (e.g., output from) the object and relaying it to one or more image sensors configured for time delay and integration (TDI) imaging; and scanning the object relative to the object-facing optical component, or the object-facing optical component relative to the object, wherein relative motion of the object and object-facing optical component during the scan is synchronized to the time delay and integration (TDI) imaging by the one or more image sensors such that a scanned image of all or a portion of the object is acquired by each of the one or more image sensors. In some instances, the illumination pattern is scanned across the object, where the scanning pattern is synchronized to the TDI imaging by the one or more image sensors to acquire the scanned image of all or a portion of the object. In some instances, the speed and the direction of the scanning is synchronized to the TDI imaging. In some instances, the scanning comprises moving the illumination pattern, moving the object, or both.

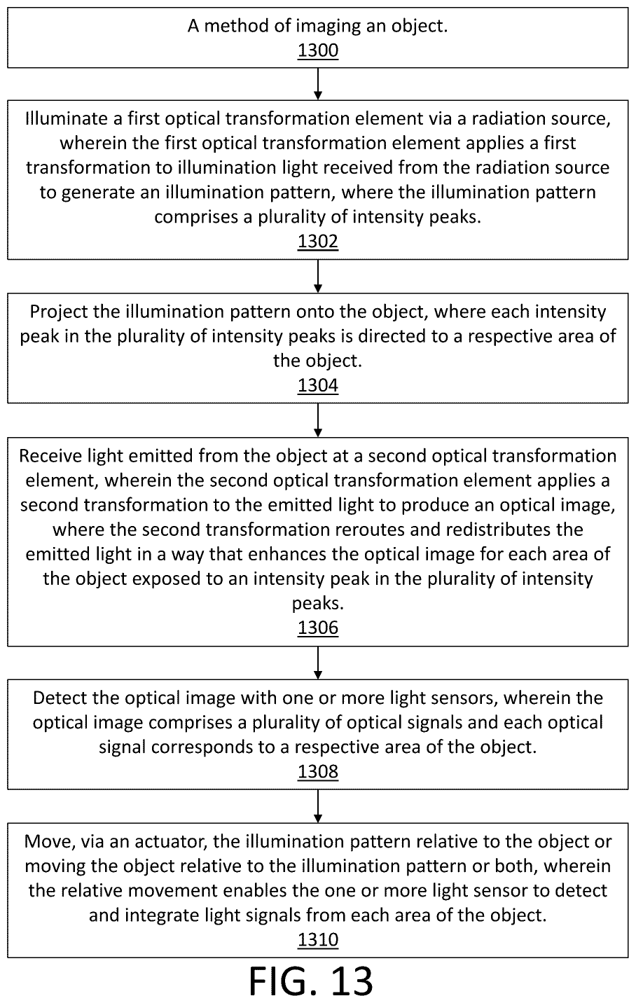

FIG. 13 provides a flowchart illustrating an example method of imaging an object 1300, in accordance with some implementations described herein. In step 1302, a first optical transformation device is used to transform light provided by a radiation source to generate an illumination pattern comprising a plurality of illumination intensity peaks. In step 1304, the patterned illumination is directed to the object being imaged (e.g., using a projection optical assembly), where each illumination intensity peak (or illumination intensity maxima) is directed to a corresponding point or location on the object. In step 1306, light that is reflected, transmitted, scattered, or emitted by the object in response to being illuminated by the patterned illumination is collected and directed to a second optical transformation device that applies a second optical transformation to the collected light and reroutes and redistributes in a way that compensates for a spatial shift that would have been observed by each individual image sensor pixel of a TDI image sensor in an otherwise identical imaging system that lacked the second optical transformation device (i.e., the second optical transformation device produces a transformed optical image). In step 1308, the transformed optical image is focused on one or more image sensors configured for TDI imaging that detect and integrate optical signals to acquire an enhanced resolution image of the object. In step 1310, which is performed in parallel with the image acquisition in step 1308, an actuator is used to move the object relative to the illumination pattern (and imaging optics), or to move the illumination pattern (and imaging optics) relative to the object, so that relative movement of the object and the pixel-to-pixel transfer of accumulated photoelectrons in the one or more TDI image sensors is synchronized, and light arising from each point on the object is detected and integrated to produce an enhanced resolution, high SNR image.

In some instances, only a portion of the object may be imaged within a scan. In some instances, a series of images is acquired, e.g., through performing a series of scans where the object is translated in one or two dimensions by all or a portion of the field-of-view (FOV) between scans, and the series of scans is aligned relative to each other to create a composite image of the object having a larger total FOV.

### Increased Illumination Density Configurations

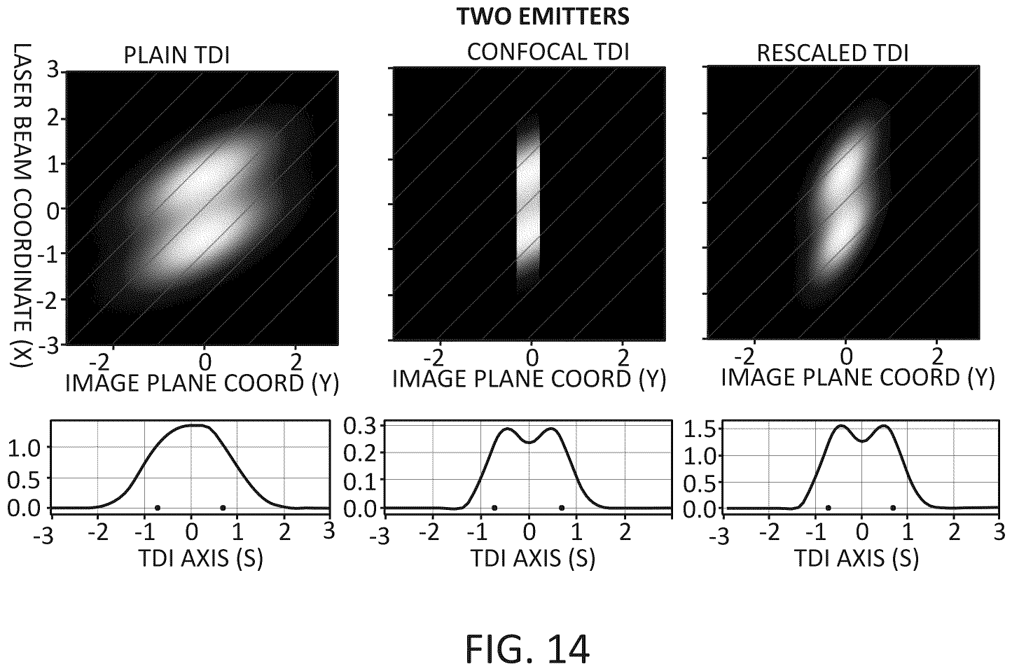

In addition to increased scanning rate, imaging and/or sequencing throughput can also be raised by increasing the density of analytes on a substrate. However, denser arrays of analytes require concomitantly increased imaging resolution. As illustrated in e.g., FIG. 14 and Example 1, TDI imaging with optical transformation can improve resolution to a degree. By utilizing excitation illumination that is directed to a substrate external to an objective lens of an imaging system, resolution can be improved even more. That is, an effective illumination NA greater than the NA permitted by an objective can be used to increase the amount of illumination directed to an analyte. This can improve resolution by a) increasing the density of illumination points (e.g., illumination intensity maxima) and b) decreasing the size of illumination points.

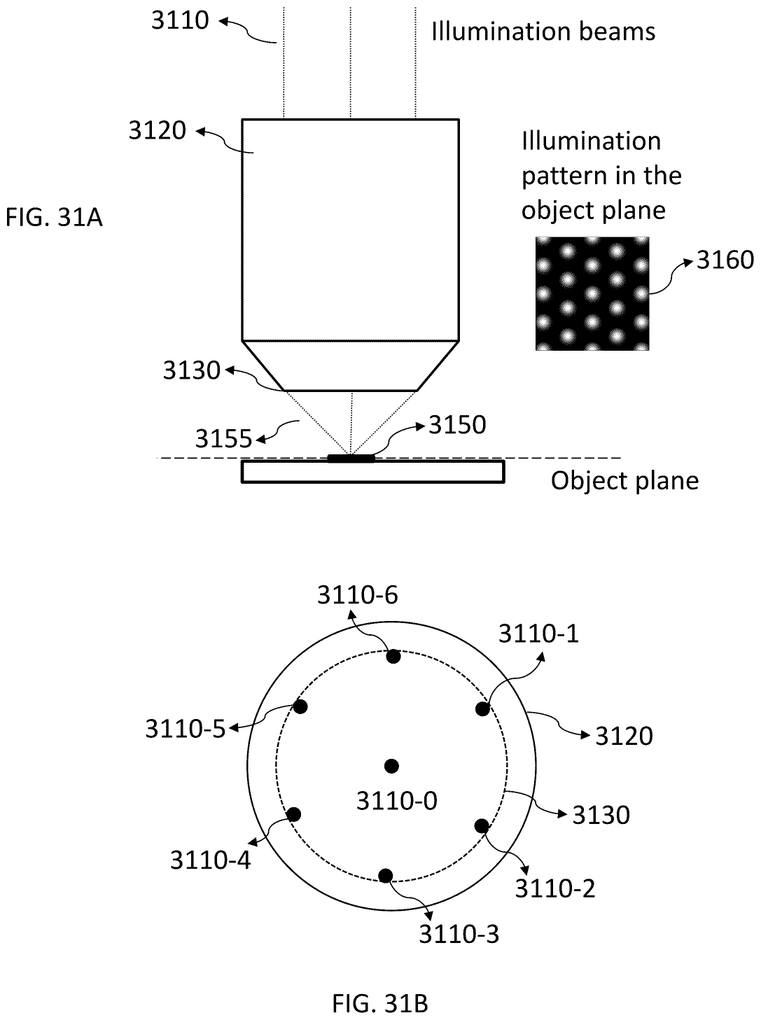

FIG. 31A illustrates an example illumination configuration that supports the current CoSI system. Input illumination 3110 passes through objective system 3120, including objective lens 3131 to provide illumination to sample 3150 on the object plane. FIG. 31B shows a top schematic representation of the system shown in FIG. 31A. The object plane and sample are omitted for simplicity. As shown in FIG. 31B, a multiple beam set up, including input beams 3110-1, 3110-2, . . . , 3110-6 and an optional center beam 3110-0, collectively creates the structured illumination pattern 3160 via interference. In this example, all input beams pass through the objective system 3120. Positions of the input beams are limited by the size of lens 3130. As a result, the sample is only illuminated in an area within cone 3155. This area correlates with the numerical aperture of lens 3130. In some embodiments, the area can be modified by filling the space between sample 3150 and lens 3130 with an immersion fluid having a refractive index higher than that of air. Such immersion fluids include but are not limited to any one or combinations of water, oil (e.g., vegetable oil or oil derived from petroleum), glycerol, glucose, silicone oil, an organic solvent (e.g., carbon disulfide, benzene, carbon tetrachloride), and polyethylene glycol (PEG).

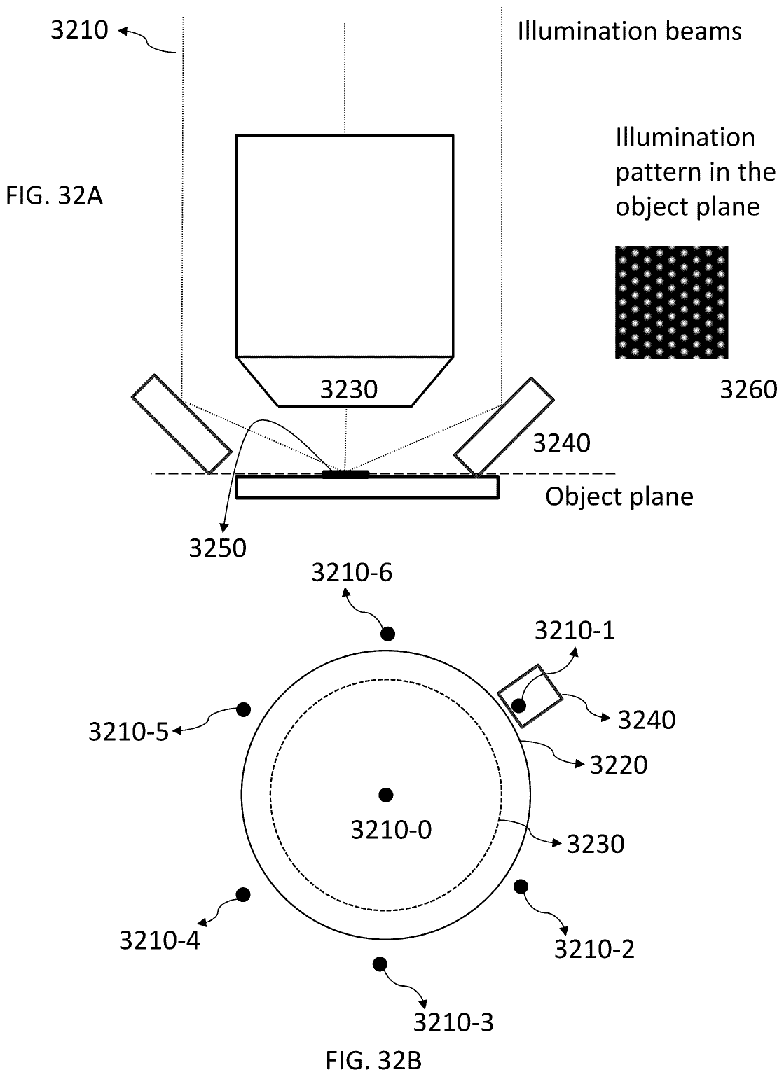

FIG. 32A illustrates an example of illumination configuration that supports a modified form of CoSI where discrete collimated beams at angles outside of the numerical aperture of objective lens 3230 are directed onto sample 3250 via optical element 3240. This configuration is referred to as external continuous structured illumination (X-CoSI or xCoSI). FIG. 32B shows a top schematic representation of the system shown in FIG. 32A. For simplicity, the object plane and sample are omitted. Also, for simplicity, only one example optical element 3240 is shown being associated with input beam 3210-1.

As seen in FIG. 32A, the input illumination 3210 (e.g., the external illumination light beams) has an incident angle that is much greater than the numerical aperture of lens 3230, when redirected. In some embodiments, the incident angle can be close to 90 degrees. As a result, a denser illumination pattern can be achieved (FIG. 32A, right). In some embodiments, an individual optical element 3240 is associated with each input beam (e.g., 3210-1, 3210-2, etc.) that does not pass through the objective lens system (e.g., external beams). In some embodiments, the same optical element can be used to redirect two different input light beams. For example, 2, 3, 4, 5 or more optical elements, or a single optical element, can be used to direct 6 input beams as shown in FIG. 32B. In some embodiments, a multi-faceted prism can be used to direct multiple beams towards the sample. For example, additional elements (one or multiple) can be used to create the illumination beams (e.g., mutually coherent beams) and to route them towards the multi-faceted prism that directs the beams onto the sample.

In some situations, these individual optical elements 3240 can be used to provide active phase compensation for each input light beam (e.g., beamlet). In some cases, individual optical elements 3240 can modify the shape, angle, size, and relative positioning of the input light beams. In some cases, the optical elements comprise immersion couplers (e.g., prism couplers). In some cases, the optical elements are in contact with the object (e.g., rest on the surface of the object plane or contacted to the surface via a liquid layer). In some cases, the optical elements may be incorporated into the objective jacket or the objective window. In some cases, the optical elements may be mirrors.

In some cases, the input light beams are produced by directing illumination from a radiation source through an optical transformation device (e.g., a diffraction grating, MLA, etc.). In some cases, the illumination light may be transmitted through an objective element (e.g., through the objective jacket) but external to the objective lens.

To provide optimal resolution improvement, each external beam will be incident on a same sized field of view of the object (e.g., a substrate). In some cases, each field of view comprises an area at least 10 μm×10 μm, 10 μm×100 μm, 100 μm×100 μm, 10 μm×1 mm, 100 μm×1 mm, 1 mm×1 mm, 10 μm×10 mm, 100 μm×10 mm, 1 mm×10 mm, or 10 mm×10 mm. By way of specific example, each external beam field of view may be 2.6 mm×10 μm.

In some cases, central input light beam 3210-0 may be omitted. The central illumination light beam primarily impacts axial resolution (e.g., z-axis resolution), where the external light beams primarily influence lateral resolution (e.g., xy-axis resolution). In some cases, axial resolution is less important than lateral resolution (e.g., when the analyte to be analyzed is substantially planar or substantially a point), and the central input light beam may not be used (e.g., only external input light may be required). In other cases, axial resolution may be more essential, and a central input light beam may be used to improve overall resolution. For example, where the analyte is a cell, tissue, or other sample with three-dimensional characteristics, increased axial resolution may be desired.

In some cases, a central light beam may be beneficial for suppressing fluorescence background. In addition, a central light beam may help suppress unwanted fringes in the interference pattern, where fringes are a product of a ratio of central beam to external beam amplitudes. In the interference pattern provided by the illumination light, only primary fringes (e.g., the regular grid of illumination maxima in the interference pattern). Additional, unwanted fringes can negatively impact the contrast of images.

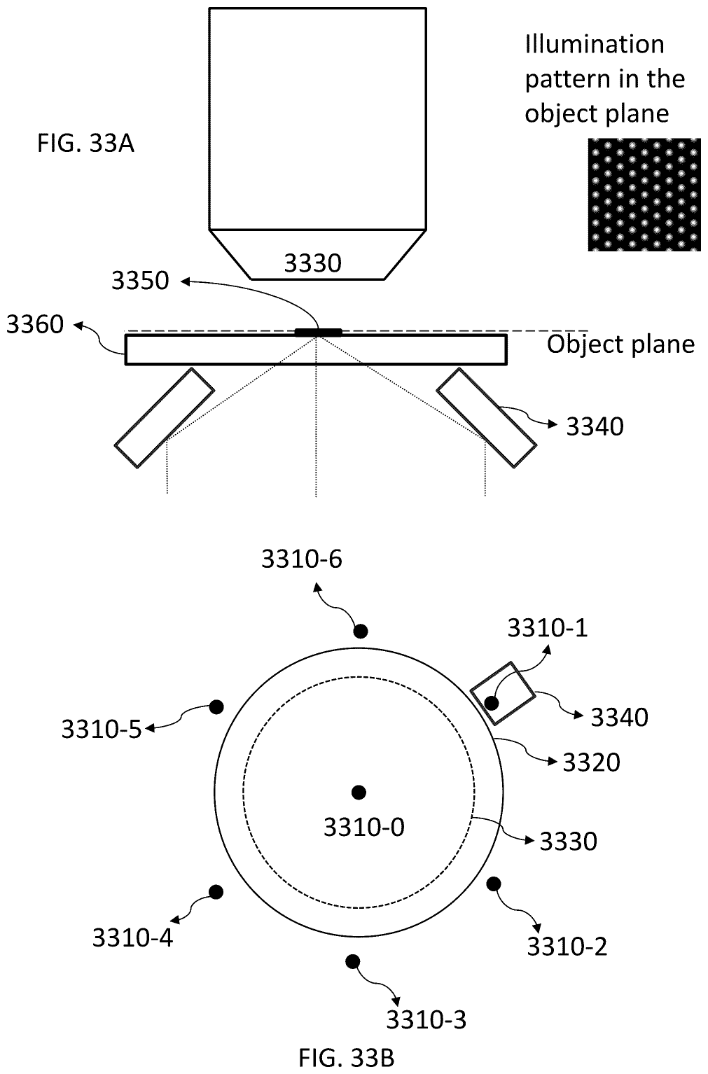

FIG. 33A illustrates another example illumination configuration that supports X-CoSI where input illumination 3310, in the form of discrete collimated beams at angles greater than the numerical aperture of objective lens 3330, is directed onto sample 3350 as back illumination via optical element 3340. In a back illumination configuration, the input light may be provided from a first surface of the substrate (sample platform 3360) which opposes a second surface of the substrate that comprises the sample 3350, such that the light travels through the depth of the substrate prior to intersecting the sample. Incoming beams are reflected and refracted before reaching sample 3350. In a configuration like this, sample platform 3360, or at least a portion thereof, comprises one or more optical elements that allow reflected light from optical element 3340 to pass through before reaching sample 3350.

Similar to the embodiment shown in FIGS. 32A and 32B, the input illumination 3310 here has an incident angle that is much greater than the numerical aperture of lens 3330, when redirected. In a back illumination configuration, all incoming beams as well as an optional center beam do not pass through the objective lens system. In some embodiments, the incident angle can be close to 90 degrees. As a result, a denser illumination pattern can be achieved (FIG. 33A, right). In some embodiments, individual optical element 3340 is associated with each input light beam (e.g., 3310-1, 3310-2, etc.). In some embodiments, the same optical element can be used to redirect two different input light beams. For example, 2, 3, 4, 5 or more optical elements, or a single optical element, can be used to direct 6 input beams as shown in FIG. 33B. In some embodiments, a multi-faceted prism can be used to direct multiple beams towards the sample. For example, additional elements (one or multiple) can be used to o create the illumination beams (e.g., mutually coherent beams) and to route them towards the multi-faceted prism that directs the beams onto the sample.

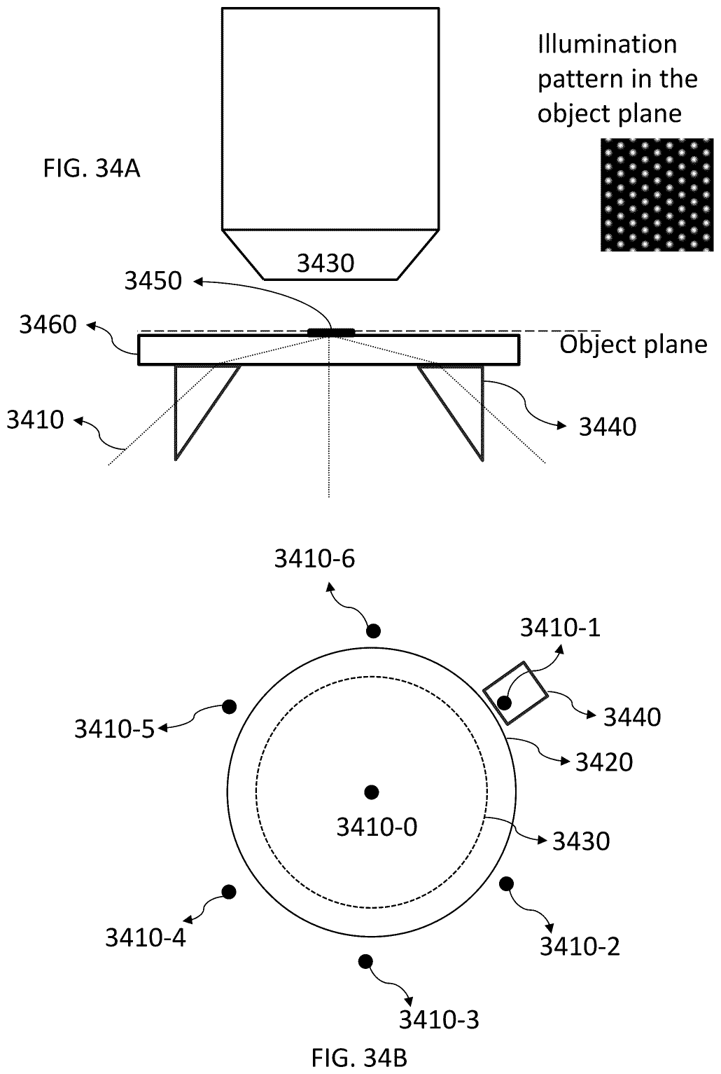

FIG. 34A illustrates another example illumination configuration that supports X-CoSI where back illumination is provided to sample 3450 via a configuration similar to that of Total Internal Reflection Fluorescence (TIRF) microscopy. Here, input illumination 3410, in the form of discrete collimated beams at angles greater than the numerical aperture of objective lens 3430, is directed onto sample 3450 as back illumination via optical element 3440. The input illumination 3410 is refracted multiple times (e.g., first through optical element 3440 and then through sample platform 3460) before reaching sample 3450.

Back illumination allows illumination optics to access a sample without any obstruction. In some cases, when back illumination is used, the substrate may be transparent or partially transparent. XCoSI illumination as illustrated in FIGS. 33 and 34 differs from conventional TIRF in that xCoSI illumination is patterned (e.g., structured). However, xCoSI may be compatible with TIRF, where illumination would comprise evanescent light.

In the TIRF-like embodiments, when redirected, input illumination 3410 has an incident angle that is much greater than the numerical aperture of lens 3430. This is also a back illumination configuration where all incoming beams as well as an optional center beam do not pass through the objective lens system. As a result, a denser illumination pattern can be achieved (FIG. 34A, right). In addition, there is no risk of illumination light being present in the imaging pathway (e.g., the detection pathway). In some embodiments, individual optical element 3440 is associated with each input light beam (e.g., 3410-1, 3410-2, etc.). In some embodiments, the same optical element can be used to redirect two different input light beams. For example, 2, 3, 4, 5, or more optical elements, or a single optical element, can be used to direct 6 input beams as shown in FIG. 34B. In some embodiments, a multi-faceted prism can be used to direct multiple beams towards the sample. For example, additional elements (one or multiple) can be used to o create the illumination beams (e.g., mutually coherent beams) and to route them towards the multi-faceted prism that directs the beams onto the sample.

The main advantage of the embodiment shown in FIGS. 34A and 34B is that it offers an even higher effective numerical aperture (NA) of the illumination, and therefore higher resolution. In this case, the effective illumination NA is not limited by the refractive index of the immersion fluid (and/or of the objective), but by the higher refractive index of the substrate material (which is at least partially transparent for this scheme). Another advantage is that, in this configuration, the illumination light (only the tilted beams) only penetrates the near-surface layer of the immersion liquid, which can reduce background fluorescence. With TIRF, no beam exits the sample platform on the sample side.

Each of the illumination configurations are provided by way of example only. Any one or any combination of them, when applicable, can be used in combination with any other methods disclosed herein or known to one of skill in the art. Such methods include but are not limited to a photon reassignment method or a TDI detection method.

### Computing Devices and Systems

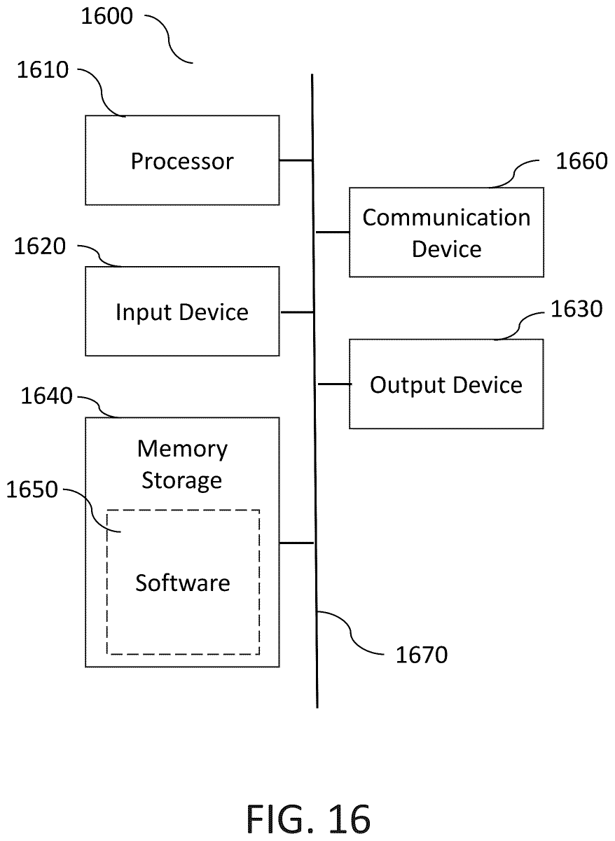

FIG. 16 illustrates an example of a computing device in accordance with one or more examples of the disclosure. Device 1600 can be a host computer connected to a network. Device 1600 can be a client computer or a server. As shown in FIG. 16, device 1600 can be any suitable type of microprocessor-based device, such as a personal computer, workstation, server, or handheld computing device (portable electronic device), such as a phone or tablet. The device can include, for example, one or more of processors 1610, input device 1620, output device 1630, storage 1640, and communication device 1660. Input device 1620 and output device 1630 can generally correspond to those described above, and they can either be connectable or integrated with the computer.

Input device 1620 can be any suitable device that provides input, such as a touch screen, keyboard or keypad, mouse, or voice-recognition device. Output device 1630 can be any suitable device that provides output for a user, such as a touch screen, haptics device, or speaker.

Storage 1640 can be any suitable device that provides storage, such as an electrical, magnetic, or optical memory including a RAM, cache, hard drive, or removable storage disk. Communication device 1660 can include any suitable device capable of transmitting and receiving signals over a network, such as a network interface chip or device. The components of the computer can be connected in any suitable manner, such as via a physical bus 1670 or wirelessly.

Software 1650, which can be stored in memory/storage 1640 and executed by processor 1610, can include, for example, the programming that embodies the functionality of the present disclosure (e.g., as embodied in the devices described above).

Software 1650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1640, that can contain or store programming for use by or in connection with an instruction execution system, apparatus, or device.

Software 1650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 1600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1600 can implement any operating system suitable for operating on the network. Software 1650 can be written in any suitable programming language, such as C, C++, Java, or Python. In various embodiments, application software embodying the functionality of the present disclosure can be deployed in different configurations, such as in a client/server arrangement or through a web browser as a web-based application or web service, for example.

### EXAMPLES

### Example 1 Enhanced Resolution for Imaging of Two Emitters

FIG. 14 provides an example of the resolution improvement provided by optical transform TDI imaging systems, in accordance with some implementations described herein. Heat maps (i.e., simulated plots of image intensity as a function of laser beam coordinate (X) and image plane coordinate (Y)) are shown for two closely spaced point emitters as imaged using conventional TDI imaging (upper left), confocal TDI imaging (e.g., a confocal imaging system comprising a single pinhole aligned with the central pixel in a TDI image sensor; upper middle), and a rescaled TDI imaging system (comprising a second optical transformation device, e.g., a micro-lens array, to rescale the illumination PSF and detection PSF) as described herein (upper right). The corresponding image intensity profiles are plotted for the conventional TDI imaging system (lower left), the confocal TDI imaging system (lower middle), and the rescaled TDI imaging system (lower right). As can be seen, the rescaled TDI imaging system is capable of producing an image having image resolution that is comparable to (or better than) that obtained using a confocal TDI imaging system, and both the confocal TDI imaging system and rescaled TDI imaging system produce images having a significantly higher image resolution that that obtained using a conventional TDI imaging system. Furthermore, the rescaled TDI image has higher signal (and corresponding improvements in SNR and contrast) than the image obtained using confocal TDI imaging (see the relative intensity scales for the intensity profiles plots), as a significant portion of emitted light is blocked by the pinhole in the latter instrument.

### Example 2 Image Resolution Versus Imaging Technique

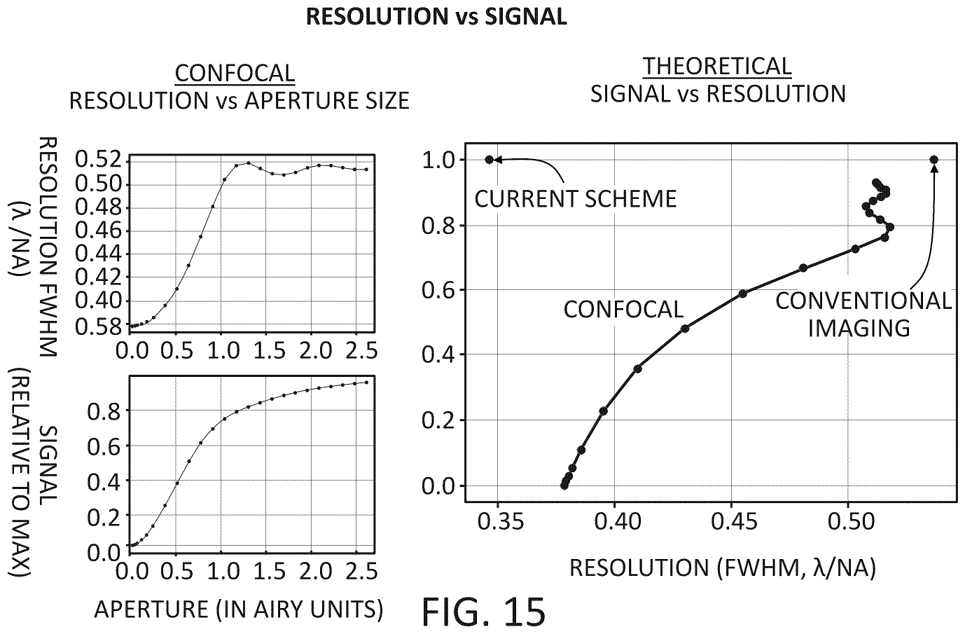

FIG. 15 illustrates the relationship between signal and resolution in different imaging methods. The left-hand panel in FIG. 15 provides plots of image resolution (FWHM of the effective point spread function in units of ANA) versus aperture size (in Airy units, i.e., where an Airy unit is the diameter of the first zero-intensity ring around the central maximum peak of a diffraction-limited Airy pattern) and signal intensity (relative to maximum signal) versus aperture size (in Airy units) for a confocal imaging system. As can be seen, the image resolution that can be achieved using a confocal imaging system reaches a maximum value of about 0.52 at an aperture size corresponding to about 1.25 Airy units and then plateaus. The signal strength that can be achieved using a confocal imaging system initially increases sharply as aperture size increases, but then increases much more slowly for apertures larger than about 1.25 Airy units.

The right-hand panel of FIG. 15 provides a plot of the theoretical relative signal strength versus image resolution for conventional imaging, confocal imaging, and the disclosed optical transformation imaging systems. Conventional imaging systems are limited by diffraction to an image resolution of about 0.54 on this scale at maximal signal strength. Confocal imaging systems can achieve image resolutions ranging from about 0.52 (at larger apertures) to about 0.38 (at smaller apertures), but with a significant corresponding loss of signal strength. The optical transformation imaging systems described herein can achieve image resolution of less than about 0.35 while maintaining high signal strength.

### Example 3 Confocal Structured Illumination (CoSI) Fluorescence Microscopy

High resolution and fast speed are essential for some applications of high-throughput imaging in optical microscopy. The spatial resolution of optical microscopy is limited because of the available numerical aperture (NA) options, even when using optical elements that have negligible aberrations. Photon reassignment (also known as “pixel reassignment”) has been demonstrated to surpass the conventional optical resolution limit to enable resolution of fine structures in biological imaging that would otherwise be difficult to visualize. This example provides a description of confocal structured illumination (CoSI) fluorescence microscopy, a concept which combines the approaches of photon reassignment (for enhanced resolution), multi-foci illumination (for parallel imaging), and a Time Delay Integration (TDI) camera (for fast imaging using reduced irradiance to minimize photodamage of sensitive samples and dyes). Computer simulations demonstrated that the lateral resolution, measured as the full width at half maximum (FWHM) of the signal corresponding to a “point” object can be improved by a factor of approximately 1.6×. That is, the FWHM of imaged objects (e.g., beads on a surface) decreased from 0.48 μm to approximately 0.3 μm by implementing CoSI. Experimental data showed that the lateral resolution was enhanced by a factor of 1.35×, with FWHM reduced from 0.54 μm to 0.4 μm in images of 0.2 μm diameter beads.

Confocal microscopy: In a typical confocal microscope, the overall 3D intensity system point spread function (PSF) is given by:

$${H}_{all} = {H}_{i} [ {H}_{d} ⊗ P ] \qquad ( 1 )$$

Here, ⊗ denotes a 3D convolution, Hᵢ and H_{d} are illumination and detection PSFs, respectively, and P denotes the confocal pinhole, which is assumed to be infinitely thin, expressed as:

$$P ( {x}_{2} , {y}_{2} , {z}_{2} ) = D ( {x}_{2} , {y}_{2} ) δ ( {z}_{2} ) \qquad ( 2 )$$

In practice, only 2D convolutions implemented in the x-y plane are needed due to the presence of δ(z₂) (the Dirac delta function) in the definition of P. With uniform illumination on the back focal plane of the objective, the intensity PSF is governed by:

$${H}_{i} , {H}_{d} ∝ {\vert {\int }_{0}^{1} {J}_{0} ( v ρ ) {e}^{- iu {ρ}^{2} / 2} ρ d ρ \vert}^{2} \qquad ( 3 )$$

where v and u are dimensionless radial and axial coordinates:

$$v = \frac{2 π}{λ} ( \frac{a}{l} ) \sqrt{{x}_{1}^{2} + {y}_{1}^{2}} , u = \frac{2 π}{λ} {( \frac{a}{l} )}^{2} {z}_{1} \qquad ( 4 )$$

Here, λ is the wavelength of the light (λᵢ and λ_{d} are the central wavelengths of illumination and fluorescence light for the detection path, respectively), l is the effective focal length of the objective, and a is the radius of the pupil aperture.

If the confocal pinhole is on-axis and infinitely small, P becomes a Dirac delta function in three dimensions, resulting in an ideal system PSF given by:

$${H}_{all} = {H}_{i} {H}_{d} \qquad ( 5 )$$

If the infinitely small confocal pinhole is placed off-axis, the system PSF is given by:

$${H}_{all} = {H}_{i} ( {x}_{2} , {y}_{2} , {z}_{2} ) {H}_{d} ( {x}_{2} - {Δ}_{x} , {y}_{2} - {Δ}_{y} , {z}_{2} ) \qquad ( 6 )$$

where Δₓ and Δ_{y} are the offset of the pinhole with respect to the optical axis.

If the PSF of a confocal microscope with an off-axis small confocal pinhole is plotted according to Eq. (6) (assuming an illumination wavelength of 0.623 μm, a central fluorescence emission wavelength of 0.670 μm, an objective numerical aperture of 0.72, and a displacement of the confocal pinhole by 0.2338 μm), the peak of the resulting system PSF is about α=0.46 of the displacement of the confocal pinhole (0.11 μm is the offset of the peak in the confocal trace whereas 0.2338 μm is the peak of the detection trace). The full width at half maximum (FWHM) is improved to 0.316 μm, compared to 0.48 μm for detection PSF and 0.44 μm for illumination PSF in comparison to a confocal microscope with an on-axis small confocal pinhole.

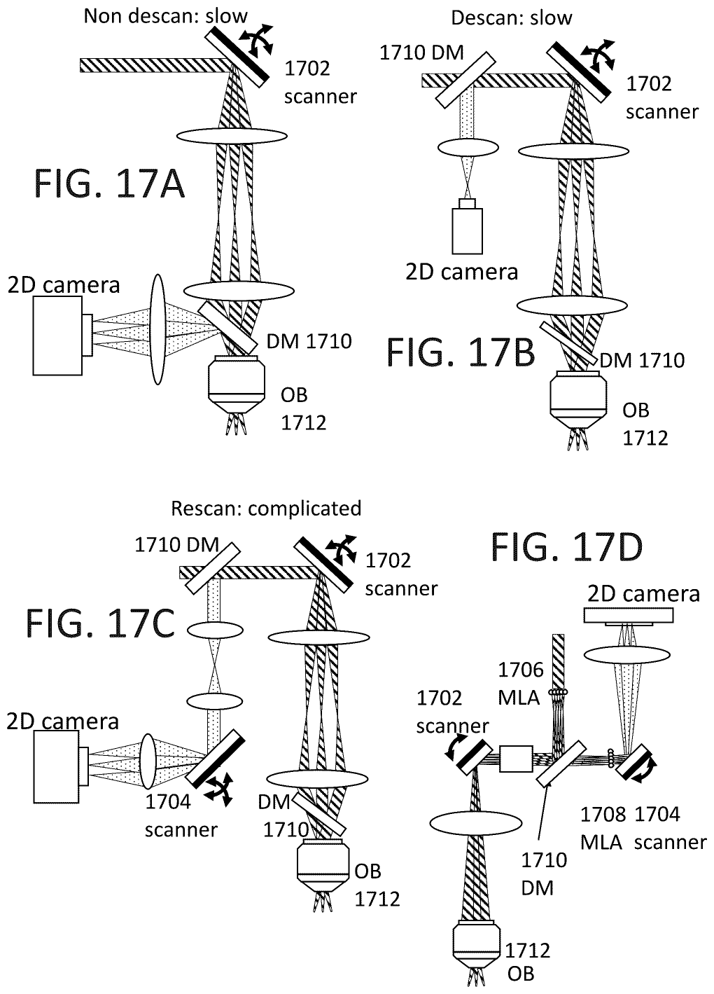

Photon reassignment and CoSI: There are several strategies that may be used to implement photon reassignment for resolution improvement. These strategies belong to two main categories: digital approaches (e.g., as illustrated in FIG. 17A and FIG. 17B) and optical approaches (e.g., as illustrated in FIG. 17C and FIG. 17D). Each of the optical systems illustrated in these figures may comprise a light source (e.g., a laser; not shown), one or more scanners 1702 and 1704 (e.g., galvo-mirrors), one or more dichroic mirrors (DM) 1710, at least on objective (OB) 1712, at least one 2D camera, and one or more additional lenses (e.g., field lenses, tube lenses, etc.). In some instances, an optical system (such as that illustrated in FIG. 17D) may comprise one or more micro-lens arrays (MLAs) or other optical transformation devices 1706 and 1708.

Digital methods for photon reassignment are typically slow. For both non-descanning (FIG. 17A; the detected light is not descanned by the scanner (e.g., a galvo-mirror) used to scan the illumination light across the sample) and descanning strategies (FIG. 17B; the detected light is descanned by the same scanner used to scan the illumination light), a 2D image is acquired and processed for each scanning point and reassignment is implemented digitally. For rescanning strategies (FIG. 17C and FIG. 17D), photons are optically reassigned. Optical photon reassignment is fast, but this speed may come at a cost of increased hardware complexity. The system illustrated in FIG. 17C scans a single spot across the sample in each cycle, while the system illustrated in FIG. 17D scans multiple illumination light foci (generated, in this example, using micro-lens array 1706) across the sample at the same time for parallel imaging with greater speed (with optical photon reassignment performed via the positional adjustment of micro-lens array 1708). These approaches, which comprise scanning the illumination light across the sample, descanning, and then rescanning the detected light, may be complicated to implement. For example, they can require at least a pair of scan lenses (e.g., the lenses adjacent to scanners 1702 and 1704 in FIG. 17C and FIG. 17D) to relay x-scanned light and y-scanned light to the back focal plane of the objective, and it can be challenging to design and manufacture a scan lens pair for a large field-of-view (FOV). In fact, each relay in the optical system adds to the complexity and cost of the system. Moreover, it may be difficult to match the primary scanner (e.g., galvo-mirror 1702 in FIG. 17C and FIG. 17D), which scans the illumination light and descans the detected light, with the secondary scanner (e.g., galvo-mirror 1704 in FIG. 17C and FIG. 17D) which rescans the detected light to the camera.

To achieve accurate optical photon reassignment, the camera and the sample typically must be kept stationary relative to each other. Thus, one strategy for achieving this goal with non-stationary samples, in addition to rescanning, is to move the camera at a speed matched to that of the sample, which greatly simplifies the imaging system. This method is also compatible with the use of a TDI camera to compensate for constant, linear relative motion between the sample and camera. Matching camera and sample motion also leverages a TDI camera's capabilities for increasing imaging throughput while reducing the level of irradiance required.

In a non-descanned confocal microscope (FIG. 17A), the intensity distribution in front of the camera is:

$$U ( {x}_{2} , {y}_{2} , {x}_{1} , {y}_{1} ) = \int \int {H}_{i} ( {x}_{0} - {x}_{1} , {y}_{0} - {y}_{1} ) S ( {x}_{0} , {y}_{0} ) {H}_{d} ( {x}_{0} - {x}_{2} , {y}_{0} - {y}_{2} ) {dx}_{0} {dy}_{0} \qquad ( 7 )$$

where (x₁, y₁) is the scanning position of the illumination light, and (x₀, y₀) and (x₂, y₂) are coordinates on the sample and camera planes, respectively. The chief ray of emitted light arising at the center of the illumination spot (x₁, y₁) on the sample arrives at (x₁, y₁) in the camera space assuming that the magnification from the sample to the camera is 1x (and ignoring the negative sign). The photons arriving at (x₂, y₂) are displaced by a distance (x_{d}=x₂−x₁, y_{d}=y₂−y₁) away from the chief ray. Those photons should be reassigned to the position [xᵣ=(1−α)x₁+αx₂, yᵣ=(1−α)y₁+αy₂]. Integrating over scanning positions (x₁, y₁) over the sample yields:

$$U ( {x}_{r} , {y}_{r} ) = \int \int {H}_{all} ( {x}_{0} - {x}_{r} , {y}_{0} - {y}_{r} ) S ( {x}_{0} , {y}_{0} ) {dx}_{0} {dy}_{0} \qquad ( 9 )$$

$$Here :$$

$${H}_{all} ( x , y ) = \int \int {H}_{i} [ x - α {x}_{d} , y - α {y}_{d} ] {H}_{d} [ x + ( 1 - α ) {x}_{d} , y + ( 1 - α ) {y}_{d} ] {dx}_{d} {dy}_{d} \qquad ( 10 )$$

and the integration over the scanning position (x₁, y₁) is replaced with integration over (x_{d}, y_{d}). Eq. (10) shows that the final PSF of the photon reassignment system is the convolution between the scaled illumination PSF and detection PSF. If α=0.5, the system PSF is simplified to

$${H}_{all} ( x , y ) = {H}_{i} ( 2 x , 2 y ) ⊗ {H}_{d} ( 2 x , 2 y ) \qquad ( 11 )$$

where ⊗ indicates the convolution operation.

The system PSF in 3D is given by:

$${H}_{all} ( x , y , z ) = \int \int {H}_{i} [ x - α {x}_{d} , y - α {y}_{d} , z ] {H}_{d} [ x + ( 1 - α ) {x}_{d} , y + ( 1 - α ) {y}_{d} , z ] {dx}_{d} {dy}_{d} \qquad ( 12 )$$

Note that the convolution only takes place in the x-y plane, and that Hₐₗₗ(x, y, z) comprises multiplication in the z direction.

Simulation results: Eq. (12) enables a straightforward estimation of the system PSF with photon reassignment. However, it doesn't account for the potential crosstalk in detected signals when a multi-foci illumination pattern is used. Therefore, the simulation for the full system accounts for Fourier optics to predict actual system performance more accurately.

Lateral resolution: FIG. 18A shows a non-limiting example of CoSI microscope incorporating a TDI camera. Eliminating scanners and optical relays can significantly reduce the complexity and cost of the system. Note that there is no mechanical component for performing optical scanning in the optical path shown in FIG. 18A. The relative motion between the sample and the camera sensor is compensated for by the TDI mechanism, which moves integrated charge across the sensor with a speed that matches that of the sample motion. Multi-foci (or structured) illumination patterns are created using either micro-lens array (MLA1) or a diffractive optical element (DOE) and projected onto the sample plane through the tube lens and the objective. The second MLA (MLA2) performs the photon reassignment.

In this simulation, the magnification of the system was set to 21.1x, the NA of the objective was 0.72, the pitch of both MLA1 and MLA2 was 23 μm, and the focal lengths of MLA1 and MLA2 were 340 μm and 170 μm, respectively. The photon reassignment coefficient α was set to 0.44 (L1/L2). The excitation wavelength was 0.623 μm and the emission wavelength was 0.670 μm. The overall improvement in lateral resolution compared to that for a wide-field microscope was a factor of ˜1.6× (0.48 μm/0.3 μm).

FIGS. 18B-18E provide non-limiting examples of the phase pattern for MLA1 (FIG. 18B), the pattern of illumination light projected onto the sample plane (FIG. 18C), the phase pattern for MLA2 (FIG. 18D), and the pattern of illumination light projected onto the pupil plane (FIG. 18E), respectively. Here, A is the ratio of the phase difference and the wavelength (e.g., the wavelength used for illumination in the imaging system). The pitch of the MLAs is designed so that on the pupil plane (or back focal plane of the objective), only the zero order and the first order of the diffraction pattern produced by MLA1 are allowed to pass through the objective (see FIG. 18E; the white circle indicates the pupil diameter in this view). The first order pattern sits close to the border of the pupil aperture to maximize the illumination resolution, which in turn benefits the final system PSF according to Eq. (10). In some instances, the peak intensity positions on the pupil plane may be adjusted, e.g., by using the second order pattern of illumination intensities produced by MLA1 rather than the zero order or first order pattern. The MLAs/DOE have three functions: (1) enabling the photon reassignment (see FIG. 18F, which shows a cross-sectional view of MLA2 and the camera; L1 is the nominal distance between MLA2 and the camera sensor, and L2 is the focal length of MLA2), (2) providing parallel imaging for fast imaging throughput, and (3) to further improve the lateral resolution of the system compared to a confocal microscope with an infinitely small confocal pinhole (˜0.29 μm in CoSI vs. 0.316 μm in the confocal system).

FIG. 18G provides a plot of the normalized system PSF in the x (upper trace) and y (lower trace) directions for a wide field microscope (FWHM_{X}=0.478 μm; FWHM_{Y}=0.477 μm; the plot for the x direction is offset by 0.5 units relative to that for the y direction). FIG. 18H provides a plot of the normalized system PSF in the x (upper trace) and y (lower trace) directions for a CoSI microscope (FWHM_{X}=0.275 μm; FWHM_{Y}=0.296 μm; again, the plot for the x direction is offset by 0.5 units relative to that for the y direction). As can be seen in these plots, the CoSI system is predicted to provide narrower point spread functions and improved lateral resolution.

Zero-order power on the pupil plane: The zero-order and/or the first order diffraction patterns for the MLA or DOE may be projected on the pupil plane (e.g., by tuning the focal length). If an MLA is used, then the zero-order pattern comprises ˜76% of the total power within the pupil aperture. By using a DOE of custom design, one can tune the power contained in the zero-order pattern. As the zero-order power becomes smaller, the FWHM of the system PSF is improved as well, while the peak-to-mean-intensity ratio of the illumination pattern on the sample is also increased. If the peak irradiance is too high, fluorescent dyes may approach their saturation levels, photodamage may be induced, and/or other damage mechanisms, e.g., due to excessive heat, may be induced. Therefore, a trade-off between lateral resolution and the peak-to-mean intensity ratio may be required. But provided that the irradiance is within a safe zone, the zero-order power should be minimized.

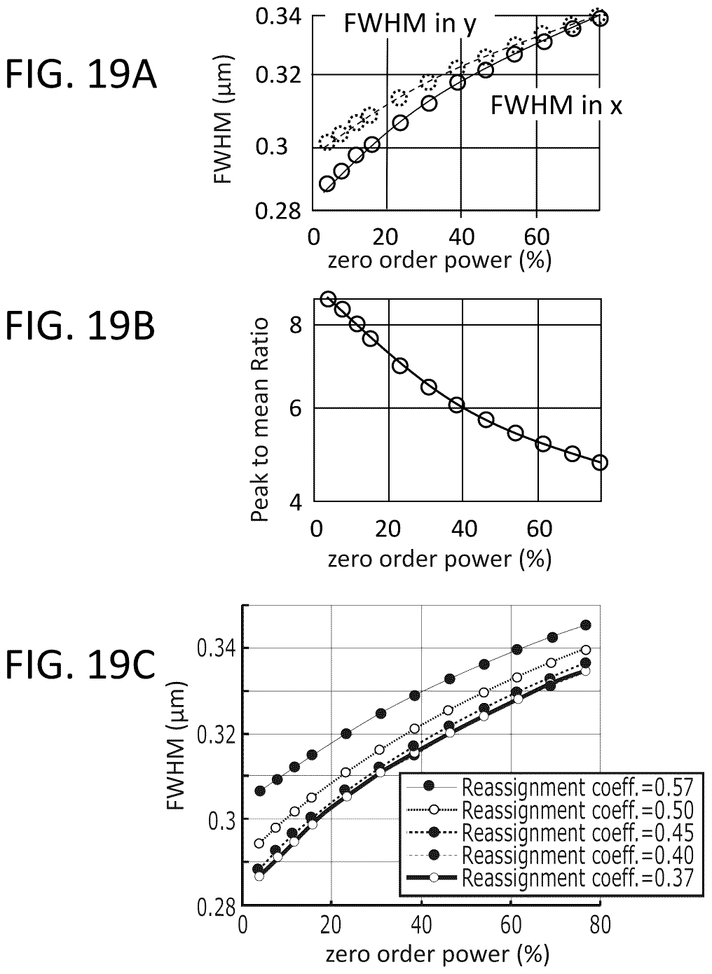

FIGS. 19A-19C illustrate these trends. FIG. 19A provides a non-limiting example of a plot of FWHM of the system PSF (in the x-direction (upper trace) and y-direction (lower trace)) as a function of the zero-order power (as a percentage of total power within pupil aperture). FIG. 19B provides a non-limiting example of a plot of peak-to-mean intensity ratio of the illumination pattern as a function of the zero-order power. FIG. 19C provides a non-limiting example of a plot of FWHM as a function of both zero-order power and photon reassignment coefficient. In the simulation, the magnification of the system was 21.1x, the NA of the objective was 0.72, the pitch of MLA1 and MLA2 was 23 μm, and the focal lengths of MLA1 and MLA2 were 340 μm and 170 μm, respectively. The excitation wavelength was 0.623 μm and the emission wavelength was 0.670 μm.

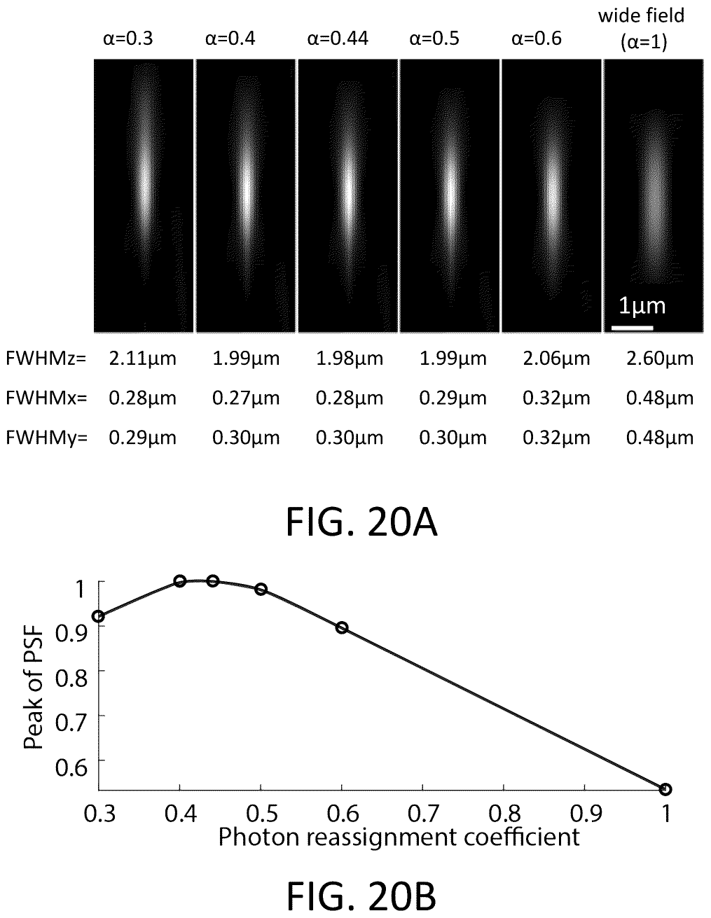

Axial resolution: The axial FWHM is a function of the photon reassignment coefficient, α. If α is within the range [0.4, 0.5], the axial FWHM is reduced by a factor of 1.3 (e.g., 2.6 μm/2 μm=1.3). FIG. 20A provides a non-limiting example of simulated system PSFs in the x, y, and z directions (projected on the x-z plane) for different values of the photon reassignment coefficient, α. FIG. 20B provides a non-limiting example of a plot of the peak value of the normalized system PSF as a function of the photon reassignment coefficient, α. Simulation parameters were the same as those described for the results described for FIGS. 18A-18H and FIGS. 19A-19C.

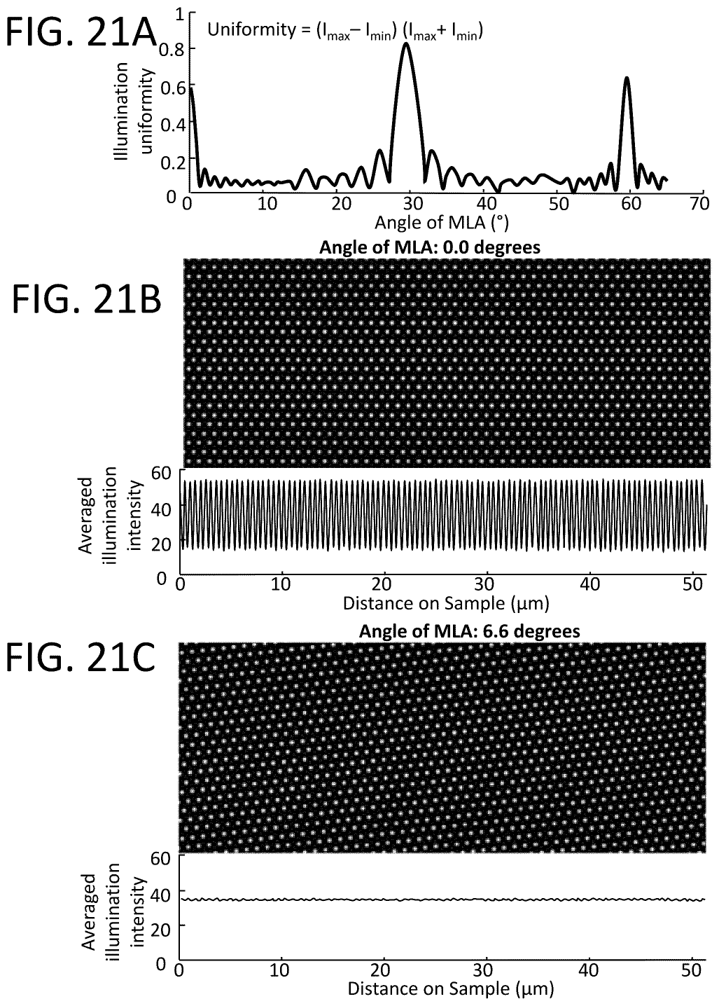

Orientation of the MLA: The orientation of the MLA affects the illumination uniformity at the sample. FIG. 21A provides a non-limiting example of a plot of illumination uniformity (defined as (Iₘₐₓ−Iₘᵢₙ)/(Iₘₐₓ+Iₘᵢₙ), where Imax and Imin are the maximum and minimum light intensities in the illumination pattern, respectively) as a function of the orientation of the MLA, and illustrates the angles that one should avoid, e.g., near 0°, 30°, 60°, etc., in order to achieve high contrast patterned illumination. For this simulation, it was assumed that 87 stages of the TDI camera are used, the pixel size was 5 μm, and the magnification from the sample to the camera was 21.1x. The other simulation parameters were the same as those for the results described for FIGS. 18A-18H and FIGS. 19A-19C.

FIG. 21B provides a non-limiting example of the illumination pattern (upper panel) and plot of the averaged illumination intensity as a function of distance on the sample (lower panel) for an MLA orientation angle of 0.0 degrees (e.g., no tilting or rotation of the second optical transformation element relative to the x and y axes of the image sensor pixel array).

FIG. 21C provides a non-limiting example of the illumination pattern (upper panel) and plot of the averaged illumination intensity as a function for distance on the sample (lower panel) for an MLA orientation angle of 6.6 degrees (e.g., tilting of the second optical transformation element). As described with respect to FIG. 7B, the MLA is tilted relative to the x and y coordinates of the rows and columns of pixels in the TDI image sensor.

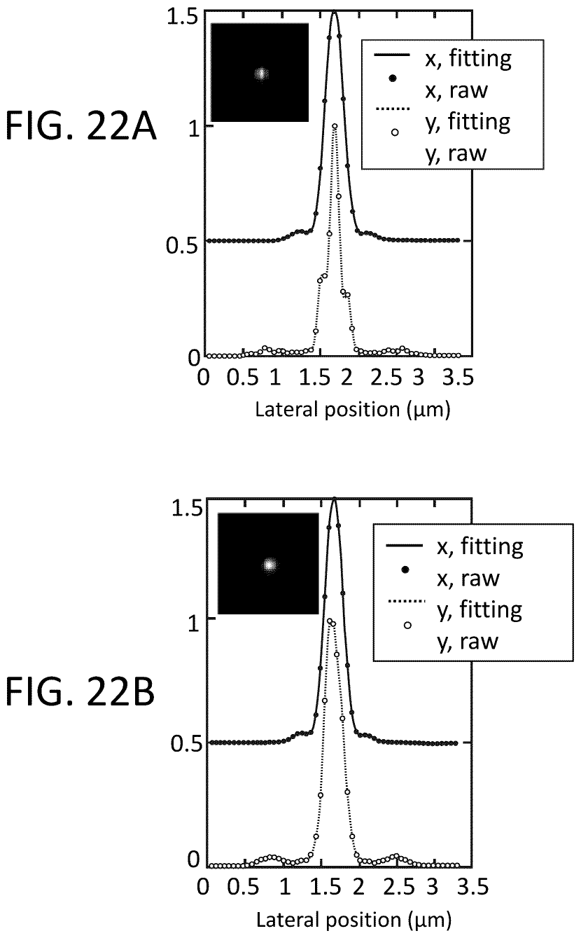

The caveat of MLA orientation and PSF measurement: In a typical optical microscope, one can acquire 3D images of well separated beads of small diameter that are significantly smaller than the diffraction-limit resolution as determined by the 3D PSF of the system. The size of the beads (e.g., lateral FWHM in x-y plane, or axial FWHM in z axis) as measured from the images of the beads for a given system 3D PSF is usually referred to as the resolution of the system. Although this relationship generally also holds for the CoSI systems described here, it may fail under certain extreme conditions. Non-limiting examples of system PSF plots are depicted in FIG. 22A (MLA2 focal length=85 μm; photon reassignment coefficient α=0.18; MLA orientation angle=6.6°; FWHM_{X}=0.146 μm; FWHM_{Y}=0.284 μm) and FIG. 22B (MLA2 focal length=85 μm; photon reassignment coefficient α=0.18; MLA orientation angle=6.0°; FWHM_{X}=0.280 μm; FWHM_{Y}=0.281 μm). The MLA orientation angle refers to the tilt of the MLA repeating pattern (see e.g., FIG. 7C), with both the first MLA and the second MLA tilted by the same amount. Although the predicted FWHM in the x direction is 0.146 μm under the set of assumptions used to generate the plot in FIG. 22A, the system is not able to resolve structures at this scale (e.g., there is an artificial apparent increase in resolution, but no real structures are revealed). This is artifact produced by using an excessively high photon reassignment (note the shoulders evident in the lower trace shown in FIG. 22A), which can be resolved by adjusting the orientation angle of the microlens array (e.g., by using MLA orientation angle=6.0° as in FIG. 22B).

Lateral misalignment of second MLA: FIG. 23A illustrates the predicted impact of lateral displacement of MLA2 on system PSF (plotted as a 2D projection on the x-y plane) for MLAs having a 23 μm pitch and suggests that lateral misalignment of up to about ±4 μm to 5 μm (e.g., ±20% of the MLA pitch) should still provide good imaging performance. FIG. 23B provides a non-limiting example of a plot of system PSF FWHM (in the x direction) as a function of the displacement of MLA2 in the CoSI microscope depicted in FIG. 18A. The lateral FWHM is worsened by about 10% with a 4 μm to 5 μm lateral misalignment.

Tolerance analysis of the distance between MLA2 and the camera: The system PSF depends on the distance between the camera sensor and MLA2, and their relative flatness (or parallelism), thus it is important to understand what level of tolerance is required for accurately setting that distance. The required tolerance depends on the magnification of the system and the focal length of MLA2. FIGS. 24A-24C show non-limiting examples of tolerance analysis results for lateral resolution, system PSF, and normalized system PSF peak intensity as a function of the separation distance between a long focal length MLA2 and the camera sensor (magnification from the sample to the camera=21.1, MLA2 focal length=170 μm, L2=220 μm and L1=96 μm (e.g., nominal distance between MLA2 and the camera sensor)). FIG. 24A: plot of lateral resolution (system PSF FWHM averaged over x and y) as a function of the distance error between MLA2 and the camera. FIG. 24B: plot of normalized peak intensity of the system PSF as a function of the distance error between MLA2 and the camera. FIG. 24C: plots of the 2D system PSF as a function of the separation distance between MLA2 and the camera in the z direction (Δz) relative to a nominal position (Δz=0). Note that the system PSF degrades more if the distance between the camera sensor and the MLA2 is smaller than the nominal value compared to the opposite direction. For a system with a magnification of 21.1×, and a MLA2 focal length=170 μm, a separation distance error of about ±25 μm (indicated by the dashed vertical lines in FIG. 24C) still maintains greater than 90% of peak PSF intensity.

If the topography of MLA2 and the sensor is known (or can be measured), in principle, a compensator (e.g., a piece of glass or an MLA2 substrate with an appropriate thickness profile) can be used to compensate for the non-flatness (or non-parallelism) of the MLA2 and camera sensor. In the semiconductor field, the tolerance of a coating thickness on a wafer can be well controlled, provided that the overall thickness of the layer is not too thick. Thus, semiconductor fabrication techniques may allow one to fabricate an appropriate compensator element.

FIGS. 25A-25C show non-limiting examples of tolerance analysis results for lateral resolution, system PSF, and normalized system PSF peak intensity as a function of the separation distance between a short focal length MLA2 and the camera sensor (magnification from the sample to the camera=21.1, MLA2 focal length=85 μm, L2=110 μm and L1=48 μm (e.g., nominal distance between MLA2 and the camera sensor)). FIG. 25A: plot of lateral resolution (system PSF FWHM averaged over x and y) as a function of the distance error between MLA2 and the camera. FIG. 25B: plot of normalized peak intensity of the system PSF as a function of the distance error between MLA2 and the camera. FIG. 25C: plots of the 2D system PSF as a function of the separation distance between MLA2 and the camera in the z direction (Δz) relative to a nominal position (Δz=0). The acceptable range for separation distance error relative to the nominal separation distance is about −10 μm to 20 μm (indicated by the vertical dashed lines in FIG. 25C), within which the PSF intensity is maintained at greater than 90% of its peak value.

Star pattern artifacts and mitigation thereof: To avoid high peak irradiance that could lead to saturation of the dye and potential damage of molecules in the sample, it can be beneficial to project illumination light foci as tightly packed as possible onto the sample while maintaining the individual illumination spots at, or even below, the diffraction limit. The maximum diffraction pattern order that's allowed to pass the pupil aperture is the 1st order, which in turn determines the smallest possible pitch that may be achieved for illumination light foci at the sample. However, the smaller the pitch, the greater the likelihood that crosstalk will occur between adjacent beamlets (arising from adjacent lenses in the microlens array) which gives rise to artifacts, e.g., star patterns, in the resulting images. Such artifacts can be mitigated by using a pinhole array positioned on or in front of the sensor.

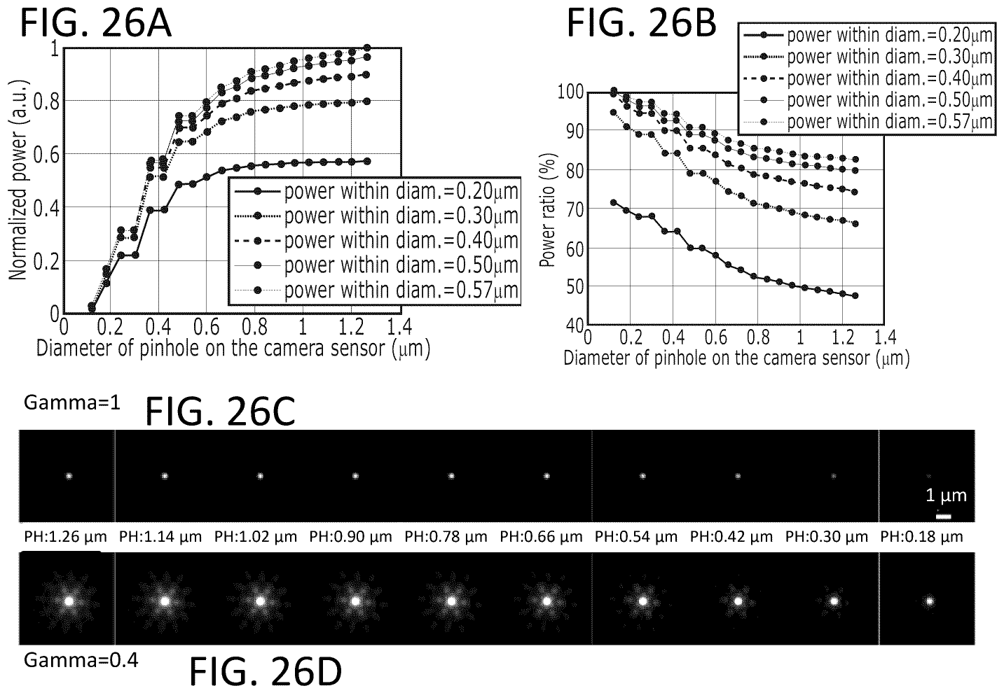

FIG. 26A provides a non-limiting example of a plot of normalized power within an aperture of defined diameter as a function of the pinhole diameter on the sensor. FIG. 26B provides a non-limiting example of a plot of the power ratio within an aperture of defined diameter as a function of the pinhole diameter on the sensor. FIG. 26C provides a non-limiting example of system PSF plotted as a function of pinhole diameter without gamma correction (Gamma=1). FIG. 26D provides a non-limiting example of system PSF plotted as a function of pinhole diameter with Gamma=0.4. The images are saturated on purpose to show the star pattern artifacts more clearly. The flat parts of curves in FIG. 26A and FIG. 26B are caused by simulation errors resulting from the use of a relatively large sampling pixel (0.06 μm on the sample) to speed up simulation. At a baseline—without using any pinhole—the power delivered within a 0.57 μm diameter aperture (FWHM=0.29 μm) is ˜82% of maximum, which is comparable to that of an Airy disk (which comprises ˜85% of total power under ideal conditions). After applying pinholes, the power delivered can extend beyond 90% (see FIG. 26B, e.g., where the diameter of the pinhole on the camera sensor has a 0.57 μm diameter). There is a tradeoff between the amount of power that can be collected and the formation of a star pattern (i.e., which can result in a decrease in effective resolution, e.g., due to crosstalk between adjacent star patterns). In practice, some amount of star pattern may be tolerated to achieve an acceptable power ratio (e.g., at least 80% of maximum).

### Example 4 CoSI Proof of Concept

Experimental results: The experimental setup used to demonstrate proof of concept of CoSI is illustrated in FIG. 27 (MLA1=first microlens array; MLA2=second microlens array; M1-M8=mirrors; BE=beam expander; DM=dichroic mirror; tube=tube lens; f_tube=tube lens focal length; OB=objective; f_OB=objective focal length). Off-the-shelf MLAs were used in the experimental setup. These off-the-shelf MLAs required the use of two relays (e.g., ‘Relay 1’ and ‘Relay 2’ in FIG. 27). In alternative schematics, these relays may be eliminated by using matched-magnification MLAs. In FIG. 27, both MLA1 (the first optical transformation element) and MLA2 (the second optical transformation element) comprise hexagonal regular arrangements of micro-lenses, with a pitch of 45 μm and a focal length of 340 μm. This experimental setup was used to image Bangs beads (Bangs Laboratories, Inc., Fishers, IN) (e.g., fluorescent europium (III) nanoparticles) to compare CoSI imaging with wide field imaging (e.g., an otherwise identical imaging system that lacks the second optical transformation device). FIG. 28 shows example images of 0.4 μm Bangs beads obtained by CoSI(upper panels) and by wide field (WF) imaging (lower panels) of a same object at multiple z positions (e.g., distance between the focal plane of the objective and the object). The CoSI images, at every z level, clearly show improved resolution, even for aggregated Bangs beads (i.e., the bright white points).

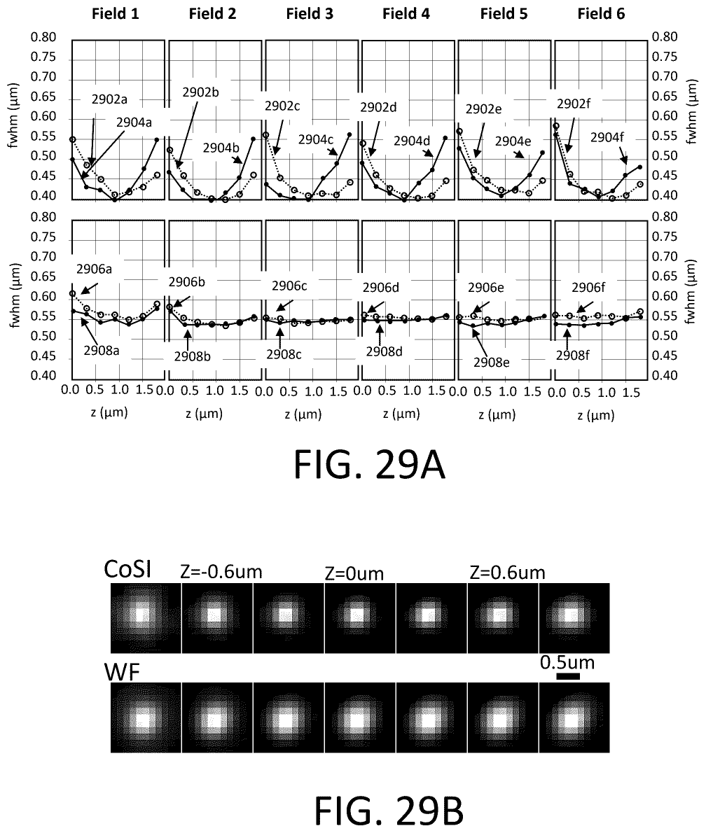

For FIGS. 29A and 29B, 0.2 μm Bangs beads were imaged. In the FIG. 29A, plots of bead signal FWHM as a function of z-axis offset are shown Lines 2902a-2902f (CoSI) and 2906a-2906f (WF) indicate average FWHM values of the bead signals in the scanning direction, and lines 2904a-2904f (CoSI) and 2908a-2908f (WF) indicate average FWHM values in a direction orthogonal to the scanning direction. Each field imaged was 40 μm, the axial step size was 0.3 μm, and the lateral pixel size was 0.1366 μm. For each data point in FIG. 29A plots, the plotted FWHM was determined from the FWHM of at least 100 Bangs beads. On average, CoSI improves the image resolution from 0.54 μm to 0.4 μm (1.35x) over a wide field imaging modality.

### Example 5 Use of a Magnification Gradient to Correct for Relative Motion

One method to compensate for rotational motion (e.g., where a rotating wafer is imaged by a stationary camera) is to create a gradient of magnification across the field-of-view of the camera's image sensor. FIG. 30A illustrates the concept of wedged counter scanning. During the camera exposure time used to acquire an image, the wafer moves a distance S₁ at radial position r₁ (e.g., the innermost edge of the sensor), and a distance of S₂ at radial position r₂ (e.g., the outermost edge of the sensor). Wedged counter scanning is achieved by creating a magnification gradient across the field-of-view of the camera such that the ratio of the magnification at r₂ to that at r₁ (magnification ratio, MR) is given by MR=S₂/S₁=r₂/r₁=1+(FOV/r₁), where FOV is the field of view along the x (radial) axis. Assuming FOV is 1.6 mm and r₂=60 mm, then the ratio of magnification at r₂ versus r₁ is MR=1.03.

FIG. 30B and FIG. 30C provide non-limiting schematic illustrations of optical designs comprising tiltable optical elements for creating and adjusting magnification gradients by changing the working distance. FIG. 30B illustrates a typical Scheimpflug optical microscope design with a tilted objective (OB) and tilted camera sensor. The magnification, M1, at r1 is given by M₁=T₁/WD₁, where T₁ is the distance between the objective and camera sensor, and WD₁ is the working distance (i.e., the separation distance between the object being imaged and the objective) at location r₁, while the magnification, M₂, at r₂ is given by M₂=T₂/WD₂, where T₂ is the nominal distance between the objective and camera sensor, and WD₂ is the working distance at location r₂. By tilting the objective and/or the camera, one can adjust the ratio M₂/M₁. FIG. 30C illustrates an extension of Scheimpflug optical microscope design that comprises an objective, tube lens, and camera where the objective, tube lens, and/or camera are tiltable. The tilt angles illustrated in the optical configurations shown in FIGS. 30B and 30C are static for a given radial position of the camera sensor but would be adjusted as the camera is positioned at different radii.

FIGS. 30E and 30F provide additional examples that illustrate the creation of magnification gradients by adjusting the working distance of the optical system. Here the focal length of the objective and tube lens were 12.3 mm and 193.7 mm, respectively. The nominal magnification is 15.75×. FIG. 30E provides a plot of the calculated magnification as a function of the working distance displacement. At the nominal distance between the objective and tube lens (e.g., when the back focal plane of the objective is superimposed with the front focal plane of the tube lens in a 4-f system configuration; for example, 194 mm+12.3 mm=206.3 mm), the system is more or less telecentric. Reducing the working distance by 0.1 μm thus only yields a change in magnification of about 1.025×. To break the telecentricity of the optical design, one can intentionally reduce or increase the distance between the tube lens and objective. FIG. 30F provides a plot of the calculated magnification as a function of the working distance displacement with the distance between the objective and tube lens reduced by 50 mm. Reducing the working distance by 0.1 mm in this case yields a change in magnification of about 1.06×.

### Example 6 Differential Triggering

Another strategy to compensate for rotational motion without modifying overall sensor size is to use a sensor that is modularized. For instance, each sensor module may be configured to scan, or to collect frames (e.g., clock or trigger), at a different rate. Several individual sensor modules, which may or may not be in a single line, can be disposed at differing radii from a rotational axis (e.g., of the sensor and/or of the surface), and each sensor module may be configured to clock at an independent rate based on its radial position from the rotational axis. That is, the trigger rate of each sensor may be correlated to the tangential velocity of the respective portion of the field of view the sensor is scanning. For example, a sensor module disposed closer to the rotational axis may be clocked slower than a module disposed farther from the rotational axis. This method can reduce blurring at both the scanning path of smallest radial distance arc path and the largest radial distance arc path.

### Example 7 Open Substrate Processing Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. A sample processing system may comprise a substrate, and devices and systems that perform one or more operations with or on the substrate. The sample processing system may permit highly efficient dispensing of analytes and reagents onto the substrate. The sample processing system may permit highly efficient imaging of one or more analytes, or signals corresponding thereto, on the substrate. Substrates, detectors, and sample processing hardware that can be used in the sample processing system are described in further detail in U.S. Pat. Pub. Nos. 2020/0326327A1, 2021/0079464A1, and 2021/0354126A1, and International Pat. Pub. No. WO2022/072652A1, each of which is entirely incorporated herein by reference.

### Substrates

An open substrate may be a solid substrate. The substrate may entirely or partially comprise one or more materials (e.g., rubber, glass, silicon, metal, ceramic, plastic, etc.). The substrate may be entirely or partially coated with one or more layers of a metal, an oxide, a photoresist, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. A surface of the substrate may be modified to comprise active chemical groups, such as amines, esters, hydroxyls, epoxides, and the like, or a combination thereof, or these may be added as an additional layer or coating to the substrate. The substrate may have the general form of a cylinder, a cylindrical shell or disk, a rectangular prism, or any other geometric form.

The substrate may comprise a planar or substantially planar surface. The surface may be textured or patterned, where the texture or pattern may be regular or irregular. For example, the substrate may comprise grooves, troughs, hills, pillars, wells, cavities (e.g., micro-scale cavities or nano-scale cavities), and/or channels. The substrate may have regular or irregular geometric structures (e.g., wedges, cuboids, cylinders, spheroids, hemispheres, etc.) above or below a reference level of the surface. In some instances, the textures and/or patterns of the substrate may define at least part of an individually addressable location on the substrate.

The substrate may comprise a plurality of individually addressable locations. The locations on one or more surfaces of the substrate are physically accessible for processing (e.g., placement, extraction, reagent dispensing, seeding, heating, cooling, or agitation). The locations may be digitally accessible (e.g., locations may be located, identified, and/or accessed electronically or digitally for indexing, mapping, sensing, associating with a device (e.g., detector, processor, dispenser, etc.)). In some cases, the locations may be defined by physical features of the substrate (e.g., on a modified surface) to distinguish from each other and from non-individually addressable locations. In some cases, the locations may be defined digitally (e.g., by indexing) and/or via the analytes and/or reagents that are loaded on the substrate (e.g., the locations at which analytes are immobilized on the substrate). Each of the plurality of individually addressable locations, or each of a subset of the locations, may be capable of immobilizing thereto an analyte (e.g., a nucleic acid, a protein, a carbohydrate, etc. from a biological sample) or a reagent (e.g., a nucleic acid, a probe molecule, a barcode molecule, an antibody molecule, a primer molecule, a bead, etc.) directly or indirectly (e.g., via a support, such as a bead).

The substrate may have any number of individually addressable locations, for example, on the order of 1, 101, 102, 103, 104, 105, 106, 107, 108, 109, 1010, 1011, 1012, 1013 or more locations. A location may have any size. In some cases, a location may have an area of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 7, 8, 9, 10 square microns (μm²), or more. A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). For example, a first location type may comprise a first surface chemistry, and a second location type may lack the first surface chemistry.

Individually addressable locations may be distributed on the substrate with a pitch determined by the distance between the center of a first location and the center of the closest or neighboring individually addressable location(s). Locations may be spaced with a pitch of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5, 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, or 10 microns (μm). In some cases, the pitch between two locations may be determined as a function of a size of a loading object (e.g., bead). For example, where a bead has a maximum diameter, the pitch may be at least about that maximum diameter.

In some cases, the individually addressable locations may be segregated or indexed, e.g., spatially. Data (e.g., optical signals) corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some cases, the individually addressable locations are indexed by physically demarcating part of the surface, depositing a topographical mark, depositing a sample (e.g., a control nucleic acid sample), depositing a reference object (e.g., reference bead that always emits a detectable signal during detection), and the locations may be indexed with reference to such demarcations.

The substrate may be rotatable about an axis, referred to herein as a rotational axis. The rotational axis may or may not be an axis through the center of the substrate. The systems, devices, and apparatus described herein may further comprise an automated or manual rotational unit configured to rotate the substrate. The rotational unit may comprise a motor and/or a rotor. For instance, the substrate may be affixed to a chuck (such as a vacuum chuck). The substrate may be rotated at a rotational speed of at least about 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. The substrate may be configured to rotate with different rotational velocities during different operations described herein, for example with higher velocities during reagent dispense and with lower velocities during analyte loading and imaging operations. The substrate may be configured to rotate with a rotational velocity that varies according to a time-dependent function, such as a ramp, sinusoid, pulse, or other function, or combination thereof.

In some cases, the substrate may be movable in any vector or direction. For example, such motion may be non-linear (e.g., in rotation about an axis), linear (e.g., on a rail track), or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

### Open Substrate Detection Systems

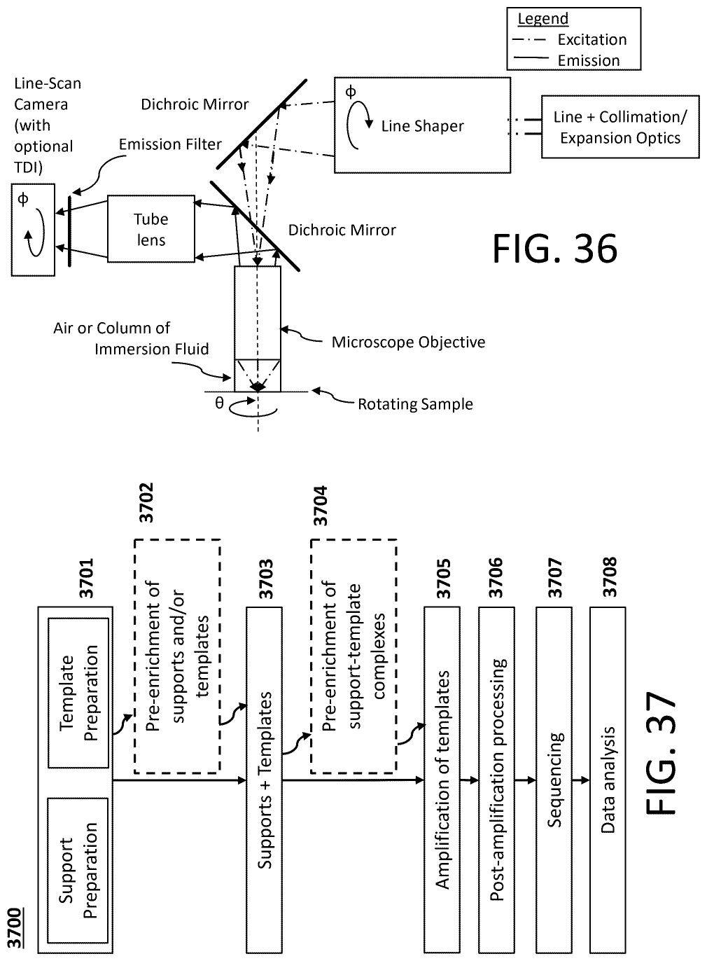

FIG. 36 shows an exemplary optical setup that may be used to scan a substrate as disclosed herein, for example a rotating substrate. An optical system comprising a detector may be configured to detect one or more signals from a detection area on the substrate prior to, during, or subsequent to the dispensing of reagents to generate an output. Signals from multiple individually addressable locations may be detected during a single detection event. Signals from the same individually addressable location may be detected in multiple instances.

The optical system may comprise one or more distinct optical paths. The one or more optical paths may comprise mirrored optical layouts. An optical path may comprise additional optical components not shown in FIG. 36. For example, an optical path may comprise additional splitting, reflecting, focusing, magnifying, filtering, shaping, rotating, polarizing, or other optical elements. An optical path may comprise an excitation path and an emission path. The excitation path and the emission path may each comprise a plurality of optical elements in optical communication with a substrate. In some cases, the excitation path comprises one or more of an excitation light source, a beam expander element, a line shaper element, a dichroic mirror, and an objective. In some cases, the emission path may comprise one or more of an objective, a dichroic mirror, a tube lens, and a detector.

The objective in the excitation path may be the same as the objective in the emission path. The objective may be an immersion objective or an air objective. The dichroic in the excitation path may be the same as the dichroic in the emission path. The dichroic may be a short pass dichroic, or the dichroic may be a long pass dichroic. In some cases, the dichroic passes the excitation light and reflects the emission light. In other instances, the dichroic reflects the excitation light and passes the emission light.

The excitation light source may be configured to emit light (e.g., coherent light). The excitation light source may comprise one or more light emitting diodes (LEDs), one or more lasers, one or more single-mode laser sources, one or more multi-mode laser sources, one or more laser diodes, a continuous wave laser or a pulsed laser, or a combination thereof. A beam of light emitted by a laser may be a Gaussian or approximately Gaussian beam, which beam may be manipulated using one or more optical elements (e.g., mirrors, lenses, prisms, waveplates, etc.). For example, a beam may be collimated. In some cases, a beam may be manipulated to provide a laser line (e.g., using one or more Powell lenses or cylindrical lenses). The excitation light source may be coupled to an optical fiber.

The line shaper may be configured to expand excitation light provided by the excitation light source along one axis. The line shaper may comprise one or more lenses (e.g., one or more cylindrical lenses). The one or more cylindrical lenses may be convex cylindrical lenses, concave cylindrical lenses, or any combination thereof. In some instances, the line shaper is positioned in a rotating mount, for example a motorized rotating mount. The rotational mount may be configured to rotate the expanded excitation light source about a central axis without substantial deviation of the central point of the excitation light source. The line shaper element may be configured to rotate about the central axis in response to, concurrent with, or in anticipation of a translation of the substrate with respect to the optical system. For example, the line shaper element may rotate about the central axis such that the axis of the expanded excitation light maintains a defined orientation with respect to the rotational axis of the substrate upon translation of the substrate with respect to the optical axis in a direction that is not directly toward or away from the rotational axis.

The beam expander may comprise one or more lenses. For example, the beam expander may comprise two lenses. The lenses may have different focal lengths. In some cases, the lens closer to the excitation light source may have a shorter focal length than the lens farther from the excitation light source. The beam expander may be configured to expand the excitation light about 2x, about 3x, about 4x, about 5x, about 10x, about 15x, or about 20x. The beam expander may be configured to collimate and/or to focus the excitation light.

The tube lens may comprise one or more lenses. For example, the tube lens may comprise two lenses. The lenses may have different focal lengths, or the two lenses may have different focal lengths. The tube lens may be configured to expand the excitation light source about 2x, about 3x, about 4x, about 5x, about 10x, about 15x, or about 20x. The tube lens may be configured to collimate the emission light and/or to focus the emission light.

The detectors may comprise any combination of cameras (e.g., CCD, CMOS, or line-scan), photodiodes (e.g., avalanche photo diodes), photoresistors, phototransistors, or any other optical detector known in the art. In some cases, the detectors may comprise one or more cameras. For example, the cameras may comprise line-scan cameras, such as TDI line-scan cameras. A TDI line-scan camera may comprise two or more vertically arranged rows of pixels. The detector may be configured to rotate with respect to a substrate to correct for tangential velocity blur, as described herein. In some cases, the detector may be configured to rotate in response to, concurrent with, or in anticipation of a translation of a substrate with respect to the optical system. For example, the detector may rotate such that the axis of the imaging field maintains a defined orientation with respect to the rotational axis of a substrate upon translation of the substrate with respect to the optical axis in a direction that is not directly toward or away from the rotational axis. The detector may be configured to rotate concurrently with a rotation of the line shaper element, such that the imaging field maintains a defined orientation with respect to the axis of the expanded excitation light. The detector may be configured to rotate independently of the line shaper element.

The optical systems of this disclosure may further comprise one or more autofocus systems. In some cases, each optical path in the optical system comprises an autofocus system. The autofocus system may comprise an autofocus illumination source configured to direct autofocus light through the objective toward the surface. The autofocus illumination source may comprise an infrared (IR) laser, e.g., a speckle-free IR laser. The autofocus light may pass through one or more of the optical elements in the optical path. The autofocus detector may be a position-sensitive detector. The autofocus light may coincide with the autofocus detector at a discrete position when the surface is in focus for an emission detector (e.g., the camera illustrated in FIG. 36). The autofocus illumination source and the autofocus detector may be configured such that a change in a position of the surface relative to the objective results in a change in position of the autofocus illumination on the autofocus detector. For example, a change in a distance between the surface and the objective or a tilt of the surface relative to the objective may cause a displacement of the autofocus illumination position on the autofocus detector. The autofocus system may send a signal to a focusing system in response to the change in position of the autofocus illumination on the autofocus detector. The focusing system may adjust the position of the surface relative to the objective such that the position of the autofocus illumination on the autofocus detector returns to the discrete position when the surface is in focus on the emission detector.

The optical systems of this disclosure may be aligned such that the excitation light and the emission light pass substantially through the center of the optical elements. For instance, the excitation light may be aligned with respect to the line shaper element such that the position of the excitation light after passing through the line shaper does not change substantially upon rotation of the line shaper. The line shaper may be rotated during alignment and the position of the excitation light source, the line shaper, or both may be adjusted to minimize motion of the position of the excitation light after passing through the line shaper upon rotation of the line shaper. In some cases, a position of the detector is aligned with respect to a rotating mount. For example, the detector is centered within the rotational mount by illuminating the center of the detector, rotating the rotational mount, and adjusting the position of the detector within the mount so that the position of the illumination does not move upon rotation of the rotational mount. In some cases, the position of the excitation light and/or the emission light is aligned at two or more points thereby defining both a position and an angle.

### Example 8 Improved Resolution with External CoSI (xCoSI)

It is possible to increase the resolution of CoSI by altering illumination conditions. Specifically, by providing illumination external to the objective (e.g., by utilizing illumination angles greater than those permitted within the confines of an objective), the density of illumination beams, and hence the resolution of the system, can be increased.

To exemplify the resolution increase, a simulation of external CoSI was performed. This simulation used the CoSI photon reassignment equations from Example 3 for both the CoSI and xCoSI. The assumed excitation and emission wavelengths were 532 nm and 570 nm, respectively. A hexagonal illumination pattern was used for CoSI and xCoSI. In the later system, this means that six illumination beams are routed external to the objective (see e.g., FIGS. 31A-34B). Additional illumination patterns are possible (e.g., 3, 4, 5, 7, 8, 9, 10 or more external illumination beams). For instance, if four external illumination beams were used, the illumination pattern would be a grid. For each imaging system, the detection NA is set to 0.72. For CoSI, the excitation NA is the same as the detection NA. For the external CoSI simulations, excitation NA can be increased (e.g., to 1.1 and 1.3, respectively).

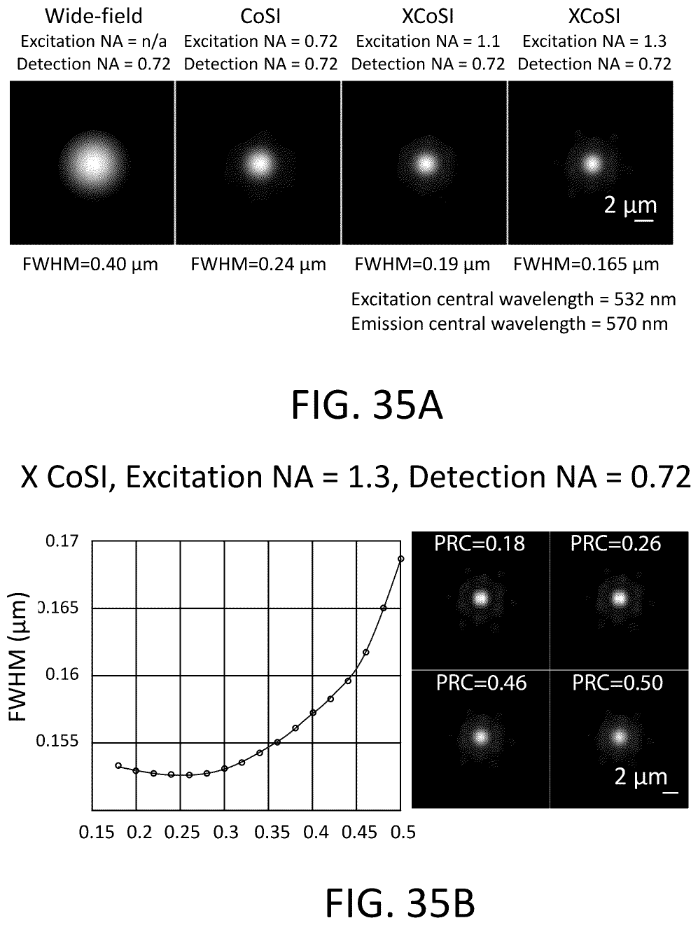

As illustrated in FIG. 35, there is a clear decrease in the observed FWHM of an imaged object (i.e., a fluorescent bead set to 50 nm in diameter in the simulation) between a widefield imaging system and a comparable CoSI imaging system and between the CoSI imaging system and comparable xCoSI systems. The widefield microscopy has resolution FWHM=0.40 μm. CoSI improves the resolution by a factor of 1.67x (to FWHM=0.24 μm) by taking advantage of an excitation NA, where the excitation light passes through the same objective. External CoSI can further improve the resolution to FWHM=0.165 μm (2.4x compared to widefield) by employing the illumination external to the objective.

### Example 9—Systems and Methods for Sequencing

The optical systems and methods described herein may be used as part of the process of sequencing nucleic acid molecules (e.g., via sequencing by synthesis) on an open substrate. It will be appreciated that these optical systems and methods may be used as part of other types of sequencing systems, and further there will be other applications beyond sequencing that may make use of these optical systems and methods (e.g., high-resolution and/or high-speed imaging).

Sequencing by synthesis: FIG. 37 illustrates an example sequencing workflow 3700, that may be performed in accordance with aspects of the present disclosure. Supports and/or template nucleic acids may be prepared and/or provided (3701) to be compatible with downstream processing (e.g., sequencing operations 3707). A support (e.g., bead) may be used to help facilitate sequencing of a template nucleic acid on a substrate. The support may help immobilize a template nucleic acid to a substrate, such as when the template nucleic acid is coupled to the support, and the support is in turn immobilized to the substrate. The support may further function as a binding entity to retain molecules of a colony of the template nucleic acid (e.g., copies comprising identical or substantially identical sequences as the template nucleic acid) together for any downstream processing. This may be particularly useful in distinguishing a colony of copies of the template nucleic acid from other colonies (e.g., on other supports) and generating sequencing signals for a plurality of template nucleic acid sequences simultaneously.

A template nucleic acid may include an insert sequence sourced from a biological sample. The template nucleic acid may further comprise an adapter sequence (e.g., for capturing by a support oligonucleotide), a primer sequence, or any other functional sequence useful for a downstream operation. Optionally, the supports and/or template nucleic acids may be pre-enriched (3702). Subsequent to preparation of the supports and template nucleic acids, the two may be attached (3703). A template nucleic acid may be coupled to a support via any method(s) that results in a stable association between the template nucleic acid and the support. Once attached, a plurality of support-template complexes may be generated. Optionally, support-template complexes may be pre-enriched (3704), wherein a support-template complex is isolated from a mixture comprising support(s) and/or template nucleic acid(s) not attached to each other.

Subsequent to attachment of the template nucleic acid molecule to the support, the template nucleic acids may be subjected to amplification reactions (3705) to generate a plurality of amplification products immobilized to the support. For example, such amplification reactions may comprise performing polymerase chain reaction (PCR), including but not limited to emulsion PCR (ePCR or emPCR), isothermal amplification (e.g., recombinase polymerase amplification (RPA)), bridge amplification, template walking, etc. Emulsion PCR methods are described in further detail in U.S. Pat. Pub. No. 2022/0042072A1 and International Pat. Pub. No. WO2022/040557A2, each of which is entirely incorporated by reference herein.

Subsequent to amplification, the supports (e.g., comprising the template nucleic acids) may be subjected to post-amplification processing (3706) to enrich for positive supports (e.g., those comprising a template nucleic acid molecule). Example methods of enrichment of amplified supports are described in U.S. Pat. No. 10,900,078, U.S. Pat. Pub. No. 2021/0079464A1, and International Pat. Pub. No. WO2022/040557A2, each of which is entirely incorporated by reference herein.

Subsequent to post-amplification processing, the template nucleic acids may be subject to sequencing (3707). The template nucleic acid(s) may be sequenced while attached to the support. Alternatively, the template nucleic acid molecules may be free of the support when sequenced and/or analyzed. In some instances, the template nucleic acids may be sequenced while attached to the support which is immobilized to a substrate. Examples of substrate-based sample processing systems are described elsewhere herein. Labeled nucleotides may comprise a dye, fluorophore, or quantum dot.

It will be appreciated that the combinations of termination states on the nucleotides, label types (e.g., types of dye or other detectable moiety), fraction of labeled nucleotides within a flow, type of nucleotide bases in each flow, type of nucleotide bases in each flow cycle, and/or the order of flows in a flow cycle and/or flow order, can be varied for different SBS methods. In cases where unterminated nucleotides are used, multiple nucleotides may be incorporated on a template in a single sequencing flow. In cases where terminated or reversibly terminated nucleotides are used, typically a single nucleotide may be incorporated on a template in a single sequencing flow. Different types of nucleotide bases may be flowed in any order and/or in any mixture of base types that is useful for sequencing. Various flow-based sequencing systems and methods are described in U.S. Pat. Pub. No. 2022/0170089A1, which is entirely incorporated herein by reference.

Subsequent to sequencing, the sequencing signals collected and/or generated may be subjected to data analysis (3708). The sequencing signals may be processed to generate base calls and/or sequencing reads. In some cases, the sequencing reads may be processed to generate diagnostics data of the biological sample, or the subject from which it was derived. While the sequencing workflow 3700 with respect to FIG. 37 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

### Example 10—High Throughput Processing Methods

An open substrate as described herein may be processed within a modular local sample processing environment. A barrier comprising a fluid barrier may be maintained between a sample processing environment and an exterior environment during certain processing operations, such as reagent dispensing and detecting. Systems and methods comprising a fluid barrier are described further in U.S. Pat. Pub. No. 2021/0354126A1, which is entirely incorporated herein by reference.

As shown in FIGS. 38A and 38B, a processing system 3800 (e.g., for use with open substrates) may comprise different operating stations (e.g., 3820a, 3802b, 3820c). For example, an operating station may comprise a chemical station (e.g., 3820a, 3820c) configured for reagent dispensing, analyte processing, and/or washing; a sample loading station, a sample storage station, or a detection station (e.g., 3820b), such as for detection of a signal or signal change. Any barrier system (e.g., 3805a, 3805b) of the processing system may be capable of traveling (e.g., along rail or track 3807) between different operating stations, thus moving an open substrate from one operating station to another. In some instances, different barrier systems may share the same rail or track or other motion path for travel between the different operating systems (e.g., as illustrated in FIGS. 38A and 38B). In such cases, the different barrier systems may be configured to move independently of each other on the same rail or track or other motion path, or to move in unison. In some instances, a respective different barrier system may move on a dedicated, separate rail or track or other motion path.

The processing system or any element thereof may be environmentally controlled. For instance, operating stations may have different local temperatures, pressures, and/or humidity. For example, a chemical station may comprise first operating conditions, and a detection station may comprise second, different operating conditions.

A barrier system may be configured to maintain a fluid barrier between a sample processing environment and an exterior environment. The barrier system is described in further detail in U.S. Pat. Pub. No. 2021/0354126, which is entirely incorporated herein by reference. A sample environment system may comprise a sample processing environment defined by a chamber and a lid plate, where the lid plate is not in contact with the chamber. While FIGS. 38A and 38B illustrates a processing system 3800 comprising three operating stations (e.g., 3820a, 3820b, 3820c) and two barrier systems (e.g., 3803a, 3803b), it will be appreciated that a processing system may have any number of operating stations and any number of barrier systems.

An operating station 3820 may have one or more operating units configured to facilitate an operation with respect to a sample or the sample environment (or local environment(s) thereof). An operating unit may protrude into the sample environment of a barrier system from the external environment. An operating unit may comprise one or more detectors (3801) configured to facilitate detection of a signal or signal change from a sample; a fluid dispenser (e.g., 3809a, 3809b) configured to facilitate reagent or fluid dispensing to a sample; an environmental unit configured to facilitate environment regulation of a sample environment; a light source, heat source, or humidity source; or any one or more sensors.

In some instances, the processing system 3800 may comprise a plurality of modular plates (e.g., 3803a, 3803b, 3803c) that may be coupled or otherwise fastened to each other to create an uninterrupted plate 3803. In some instances, each modular plate may comprise one or more operating stations (e.g., operating stations are coupled or otherwise fastened to plate 3803). In some instances, a modular plate may be detachable from another modular plate or a remainder of the plate 3803 without disturbing sample environments of respective barrier systems, such as during an operation by one or more operating units on a barrier system, while another barrier system is subject to another operation at another operating station. Beneficially, detachment of a modular plate may allow access to a sample environment, such as to load or unload a chamber, without disturbing another sample environment (e.g., contained within another barrier system).

Chambers of the present disclosure may comprise a base and side walls to define an opening that nearly contacts the plate (or lid). The side walls may be a closed continuous surface, or a plurality of adjacent (and/or adjoining) surfaces. For example, the base may comprise or be the substrate. In some instances, the base may be coupled to the substrate. The substrate may be translatable relative to the base. The substrate may be rotatable relative to the base. While examples herein describe relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion. Beneficially, relative motion between the one or more detection units in the detection station and the substrate may significantly increase detection efficiency. Additional details of detector systems, including immersion optic systems, are available in, for example, International Pat. Pub. Nos. WO2019099886, WO20200118172, WO2020186243, each of which is herein incorporated by reference in their entireties.

In some instances, an open substrate (e.g., 3830a, 3830b) is retained in the same or approximately the same physical location during processing of an analyte and subsequent detection of a signal associated with a processed analyte. The open substrate may transition between different stations by transporting a sample processing environment containing the open substrate (such as the one described with respect to the barrier system) between the different stations. One or more mechanical components or mechanisms, such as a robotic arm, elevator mechanism, actuators, rails, and the like, or other mechanisms may be used to transport the sample processing environment.

FIGS. 38A and 38B illustrate the multiplexing processing system 3800. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 3820a, 3820c) substantially simultaneously while the detection station (e.g., 3820b) is kept idle, such as illustrated in FIG. 38A. Beneficially, different operations within the system may be multiplexed with high flexibility and control. For example, one or more processing stations may be operated in parallel with one or more detection stations on different substrates in different modular sample environment systems to reduce or eliminate lag between different sequences of operations (e.g., chemistry first, then detection).

### EXEMPLARY EMBODIMENTS

Among the embodiments provided herein are:

1. A method of imaging, comprising: a) providing a substrate, wherein the substrate is substantially planar; b) illuminating a region of the substrate with one or more illumination beams, wherein the one or more illumination beams are not directed through an objective lens; and c) directing emission light from the region of the substrate to a detector through the objective lens, thereby generating a scanned image of the region of the substrate, wherein the emission light is directed through an optical transformation device prior to being received by the detector.

2. The method of embodiment 1, wherein the substrate comprises a first and second surface, wherein the first surface is closer to the objective lens than the second surface.

3. The method of embodiment 2, wherein the first surface and the second surface are parallel to each other, and wherein the substrate is positioned normal to the objective lens.

4. The method of any one of embodiments 2-3, wherein the one or more illumination beams are incident on the first surface of the substrate.

5. The method of any one of embodiments 2-3, wherein the one or more illumination beams are incident on the second surface of the substrate.

6. The method of any one of embodiments 4-5, wherein each of the one or more illumination beams are transmitted through a liquid immersion coupler prior to illuminating the substrate.

7. The method of embodiment 6, wherein the liquid immersion couplers comprise prism couplers.

8. The method of any one of embodiments 4-5, wherein each of the one or more illumination beams are reflected by a mirror coupled to the substrate prior to illuminating the substrate.

9. The method of any one of embodiments 1-8, wherein each of the one or more illumination beams illuminates a same sized field of view on the region of the substrate.

10. The method of embodiment 9, wherein the field of view comprises an area at least 10 μm×10 μm, 10 μm×100 μm, 100 μm×100 μm, 10 μm×1 mm, 100 μm×1 mm, 1 mm×1 mm, 10 μm×10 mm, 100 μm×10 mm, 1 mm×10 mm, or 10 mm×10 mm.

11. The method of any one of embodiments 9-10, wherein the field of view comprises 2.6 mm×10 μm.

12. The method of any one of embodiments 1-11, wherein the optical transformation device comprises one or more components selected from the group consisting of a micro-lens array (MLA), a diffractive optical element, a digital micro-mirror device (DMD), a phase mask, an amplitude mask, a spatial light modulator (SLM), and a pinhole array.

13. The method of any one of embodiments 1-12, further comprising providing initial illumination from a radiation source, wherein the initial illumination is directed through an additional optical transformation device to produce the one or more illumination beams.

14. The method of embodiment 13, wherein the additional optical transformation device comprises one or more components selected from the group consisting of a micro-lens array (MLA), a diffractive optical element, a digital micro-mirror device (DMD), a phase mask, an amplitude mask, a spatial light modulator (SLM), and a pinhole array.

15. The method of any one of embodiments 13-14, wherein illuminating the region of the substrate further comprises providing an additional illumination beam that is directed through the objective lens to the region of the substrate.

16. The method of embodiment 15, wherein the additional illumination beam is provided from the radiation source.

17. The method of any one of embodiments 15-16, wherein the additional illumination beam is provided by directing the initial illumination through the optical transformation device.

18. The method of any one of embodiments 15-17, wherein the additional illumination beam is directed through the center of a diffraction plane of the objective lens.

19. The method of any of embodiments 1-18, wherein the emission light is directed through an additional optical transformation device prior to being received by the detector.

20. The method of any one of embodiments 1-19, wherein each of the one or more illumination beams are directed to the region of the substrate at a respective angle relative to the objective lens.

21. The method of any one of embodiments 1-20, wherein the one or more illumination beams comprise 1, 2, 3, 4, 5, 6, 7, 8, 9, or 10 beams.

22. The method of any one of embodiments 1-21, wherein the detector comprises one or more image sensors configured for time delay and integration imaging.

23. The method of any one of embodiments 1-22, wherein the one or more illumination beams comprise an illumination pattern on the region of the substrate, wherein the illumination pattern comprises a plurality of light intensity maxima.

24. The method of embodiment 23, wherein the illumination pattern comprises an interference pattern.

25. The method of any one of embodiments 23-24, wherein the illumination pattern is uniform within the region of the substrate.

26. The method of any one of embodiments 23-25, wherein the illumination pattern is hexagonal.

27. The method of any one of embodiments 23-24, wherein the illumination pattern is not uniform within the region of the substrate.

28. The method of any one of embodiments 1-27, further comprising repeating b) and c) at a plurality of time points, thereby generating a plurality of scanned images of the substrate.

29. The method of any one of embodiments 1-28, wherein the scanned image exhibits a lateral spatial resolution that exceeds a diffraction-limited spatial resolution.

30. The method of any one of embodiments 1-28, wherein the scanned image exhibits a lateral spatial resolution improvement by a factor of better than 1.2, 1.5, 2, 2.5, 3, 3.5, 4, 4.5, 5, 5.5, or 6 times relative to an image obtained by a comparable diffraction-limited imaging system.

31. The method of any one of embodiments 1-30, wherein the scanned image comprises a fluorescence image.

32. The method of any one of embodiments 1-31, wherein the region of the substrate comprises an analyte and the emission light comprises light reflected, transmitted, scattered, or emitted by the analyte.

33. The method of embodiment 32, wherein the analyte comprises a biological molecule.

34. The method of embodiment 33, wherein the biological molecule comprises a nucleic acid molecule, a protein, a cell, or a tissue sample.

35. The method of embodiment 34, wherein the emission light corresponds to incorporation or a lack of incorporation of a nucleic acid base into a primer hybridized to the nucleic acid.

36. The method of embodiment 35, wherein the emission light corresponds to incorporation of more than one nucleic acid base into the primer.

37. The method of any one of embodiments 35-36, wherein repeating b) and c) determines a sequence of the nucleic acid.

38. The method of any one of embodiments 1-37, wherein the substrate comprises a flow cell or surface for performing nucleic acid sequencing.

39. An imaging system, comprising: a) a substrate, wherein the substrate is substantially planar; b) a projection unit that is configured to i) direct illumination light onto a region of the substrate in an illumination pattern, wherein at least some of the illumination light is not directed through an objective lens, and ii) direct emission light from the substrate to one or more sensors via an optical transformation device, wherein the one or more sensors are configured for time delay and integration imaging; and c) one or more processors that are singly or collectively configured to perform the methods of embodiments 1-38.

40. The imaging system of embodiment 39, wherein the optical transformation device comprises one or more components selected from the group consisting of a micro-lens array (MLA), a diffractive optical element, a digital micro-mirror device (DMD), a phase mask, an amplitude mask, a spatial light modulator (SLM), and a pinhole array.

41. The imaging system of any one of embodiments 39-40, wherein the emission light comprises light reflected, transmitted, scattered, or emitted by an analyte, wherein the analyte is positioned adjacent to the substrate.

42. The imaging system of any one of embodiments 39-41, wherein the projection unit is further configured to transmit illumination light through an additional optical transformation device to generate one or more illumination beams, wherein the one or more illumination beams provide the illumination pattern on the region of the substrate.

43. The imaging system of embodiment 42, wherein the additional optical transformation device comprises one or more components selected from the group consisting of a micro-lens array (MLA), a diffractive optical element, a digital micro-mirror device (DMD), a phase mask, an amplitude mask, a spatial light modulator (SLM), and a pinhole array.

44. The imaging system of any one of embodiments 39-42, wherein the illumination pattern is uniform within the region of the substrate.

45. The imaging system of any one of embodiments 42-44, wherein the one or more illumination beams are each transmitted through liquid immersion couplers prior to illuminating the region of the substrate.

46. The imaging system of any one of embodiments 42-45, wherein the optical transformation device and the additional optical transformation device comprise a plurality of harmonically modulated phase masks or harmonically modulated amplitude masks with different orientations.

47. The imaging system of embodiment 46, wherein a spatial frequency and orientation of the optical transformation device matches that of the additional optical transformation device.

48. The imaging system of any one of embodiments 42-45, wherein the optical transformation device and the additional optical transformation device comprises harmonically modulated phase masks, and wherein the optical transformation device is phase shifted relative to the additional optical transformation device.

49. The imaging system of any one of embodiments 39-43, wherein the one or more sensors comprise one or more time delay and integration (TDI) cameras, charge-coupled device (CCD) cameras, complementary metal-oxide semiconductor (CMOS) cameras, or a single-photon avalanche diode (SPAD) array.

50. The imaging system of any one of embodiments 39-49, wherein the projection unit is configured to provide illumination light at two or more excitation wavelengths.

51. The imaging system of any one of embodiments 39-50, wherein the one or more sensors are configured to detect fluorescence at two or more emission wavelengths.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-51. (canceled)

52. A method of imaging, comprising:
a. providing a substrate, wherein the substrate is substantially planar and wherein said substrate is rotating;
b. illuminating a region of the substrate with one or more illumination beams, wherein the one or more illumination beams are not directed through an objective lens; and
c. directing emission light from the region of the substrate to a detector through the objective lens, thereby generating a scanned image of the region of the substrate, wherein the emission light is directed through an optical transformation device prior to being received by the detector.

53. The method of claim 52, wherein the substrate comprises a first and second surface, wherein the first surface is closer to the objective lens than the second surface.

54. The method of claim 53, wherein the first surface and the second surface are parallel to each other, and wherein the substrate is positioned normal to the objective lens.

55. The method of claim 54, wherein the one or more illumination beams are incident on the first surface of the substrate.

56. The method of claim 55, wherein each of the one or more illumination beams are transmitted through a liquid immersion coupler prior to illuminating the substrate.

57. The method of claim 52, wherein each of the one or more illumination beams illuminates a same sized field of view on the region of the substrate.

58. The method of claim 52, wherein the optical transformation device comprises one or more components selected from the group consisting of a micro-lens array (MLA), a diffractive optical element, a digital micro-mirror device (DMD), a phase mask, an amplitude mask, a spatial light modulator (SLM), and a pinhole array.

59. The method of claim 52, further comprising providing initial illumination from a radiation source, wherein the initial illumination is directed through an additional optical transformation device to produce the one or more illumination beams.

60. The method of claim 59, wherein the additional optical transformation device comprises one or more components selected from the group consisting of a micro-lens array (MLA), a diffractive optical element, a digital micro-mirror device (DMD), a phase mask, an amplitude mask, a spatial light modulator (SIM), and a pinhole array.

61. The method of claim 52, wherein the emission light is directed through an additional optical transformation device prior to being received by the detector.

62. The method of claim 52, wherein each of the one or more illumination beams are directed to the region of the substrate at a respective angle relative to the objective lens.

63. The method of claim 52, wherein the detector comprises one or more image sensors configured for time delay and integration imaging.

64. The method of claim 52, wherein the one or more illumination beams comprise an illumination pattern on the region of the substrate, wherein the illumination pattern comprises a plurality of light intensity maxima.

65. The method of claim 64, wherein the illumination pattern comprises an interference pattern.

66. The method of claim 52, wherein the scanned image exhibits a lateral spatial resolution that exceeds a diffraction-limited spatial resolution.

67. The method of claim 52, wherein the region of the substrate comprises an analyte and the emission light comprises light reflected, transmitted, scattered, or emitted by the analyte.

68. The method of claim 67, wherein the analyte comprises a nucleic acid molecule.

69. The method of claim 68, wherein the emission light corresponds to incorporation or a lack of incorporation of a nucleic acid base into a primer hybridized to the nucleic acid molecule.

70. The method of claim 69, wherein the emission light corresponds to incorporation of more than one nucleic acid base into the primer.

71. The method of claim 69, wherein repeating b) and c) determines a sequence of the nucleic acid.
